SEQUENCE LISTING (48 sequences)

SEQ ID NO: 1 - RNA, 30 nt, Artificial Sequence
  DR stem loop
aaccccuaccaacuggucgggguuugaaac

SEQ ID NO: 2 - RNA, 30 nt, Artificial Sequence
  DR stem loop sequence
uaccccuaccaacuggucgggguuugaaac

SEQ ID NO: 3 - RNA, 30 nt, Artificial Sequence
  DR stem loop sequence
uuccccuaccaacuggucgggguuugaaac

SEQ ID NO: 4 - RNA, 30 nt, Artificial Sequence
  DR stem loop sequence
uuucccuaccaacuggucgggguuugaaac

SEQ ID NO: 5 - RNA, 30 nt, Artificial Sequence
  DR stem loop sequence
uuacccuaccaacuggucgggguuugaaac

SEQ ID NO: 6 - RNA, 30 nt, Artificial Sequence
  DR stem loop sequence
aaccccgaccaacuggucgggguuugaaac

SEQ ID NO: 7 - RNA, 30 nt, Artificial Sequence
  DR stem loop sequence
uaccccgaccaacuggucgggguuugaaac

SEQ ID NO: 8 - RNA, 30 nt, Artificial Sequence
  DR stem loop sequence
uuccccgaccaacuggucgggguuugaaac

SEQ ID NO: 9 - RNA, 30 nt, Artificial Sequence
  DR stem loop sequence
uuucccgaccaacuggucgggguuugaaac

SEQ ID NO: 10 - RNA, 30 nt, Artificial Sequence
  DR stem loop sequence
aaccccuaccaacugguagggguuugaaac

SEQ ID NO: 11 - RNA, 30 nt, Artificial Sequence
  DR stem loop sequence
uaccccuaccaacugguagggguuugaaac

SEQ ID NO: 12 - RNA, 30 nt, Artificial Sequence
  DR stem loop sequence
uuccccuaccaacugguagggguuugaaac

SEQ ID NO: 13 - RNA, 30 nt, Artificial Sequence
  DR stem loop sequence
uuucccuaccaacugguagggguuugaaac

SEQ ID NO: 14 - RNA, 30 nt, Artificial Sequence
  DR stem loop sequence
  misc_feature (1)..(1): B is one of C or G or U
baccccuaccaacuggucgggguuugaaac

SEQ ID NO: 15 - RNA, 30 nt, Artificial Sequence
  DR stem loop sequence
  misc_feature (1)..(1): N is A or C or G or U
  misc_feature (2)..(2): B is C or G or U
nbccccuaccaacuggucgggguuugaaac

SEQ ID NO: 16 - RNA, 30 nt, Artificial Sequence
  DR stem loop sequence
  misc_feature (1)..(1): N is C or A or G or U
  misc_feature (2)..(2): N is C or A or G or U
  misc_feature (3)..(3): D is A or G or U
nndcccuaccaacuggucgggguuugaaac

SEQ ID NO: 17 - RNA, 30 nt, Artificial Sequence
  DR stem loop sequence
  misc_feature (24)..(24): V is A or C or G
aaccccuaccaacuggucgggguvugaaac

SEQ ID NO: 18 - RNA, 30 nt, Artificial Sequence
  DR stem loop sequence
  misc_feature (23)..(23): V is A or C or G
  misc_feature (24)..(24): N is A or C or G or U
uuacccuaccaacuggucggggvnugaaac

SEQ ID NO: 19 - RNA, 30 nt, Artificial Sequence
  DR stem loop sequence
  misc_feature (22)..(22): V is A or C or U
  misc_feature (23)..(23): N is A or C or G or U
  misc_feature (24)..(24): N is A or C or G or U
aaccccuaccaacuggucggghnnugaaac

SEQ ID NO: 20 - RNA, 29 nt, Artificial Sequence
  DR stem loop sequence
accccuaccaacuggucgggguuugaaac

SEQ ID NO: 21 - RNA, 28 nt, Artificial Sequence
  DR stem loop sequence
ccccuaccaacuggucgggguuugaaac

SEQ ID NO: 22 - RNA, 27 nt, Artificial Sequence
  DR stem loop sequence
cccuaccaacuggucgggguuugaaac

SEQ ID NO: 23 - RNA, 31 nt, Artificial Sequence
  DR stem loop
  misc_feature (1)..(1): N is at least one additional A and/or C and/or G and/or U
naaccccuaccaacuggucgggguuugaaac

SEQ ID NO: 24 - RNA, 30 nt, Artificial Sequence
  DR stem loop
  misc_feature (7)..(7): V is A or C or G
aaccccvaccaacuggucgggguuugaaac

SEQ ID NO: 25 - RNA, 30 nt, Artificial Sequence
  DR stem loop
  misc_feature (1)..(1): B is one of C or G or U
  misc_feature (7)..(7): V is A or C or G
baccccvaccaacuggucgggguuugaaac

SEQ ID NO: 26 - RNA, 30 nt, Artificial Sequence
  DR stem loop
  misc_feature (1)..(1): N is A or C or G or U
  misc_feature (2)..(2): B is C or G or U
  misc_feature (7)..(7): V is A or C or G
nbccccvaccaacuggucgggguuugaaac

SEQ ID NO: 27 - RNA, 30 nt, Artificial Sequence
  DR stem loop
  misc_feature (1)..(1): N is A or C or G or U
  misc_feature (2)..(2): N is A or C or G or U
  misc_feature (3)..(3): D is A or G or U
  misc_feature (7)..(7): V is A or C or G
nndcccvaccaacuggucgggguuugaaac

SEQ ID NO: 28 - RNA, 30 nt, Artificial Sequence
  DR stem loop
  misc_feature (7)..(7): V is A or C or G
  misc_feature (24)..(24): V is A or C or G
aaccccvaccaacuggucgggguvugaaac

SEQ ID NO: 29 - RNA, 30 nt, Artificial Sequence
  DR stem loop
  misc_feature (7)..(7): V is A or C or G
  misc_feature (23)..(23): V is A or C or G
  misc_feature (24)..(24): N is A or C or G or U
uuacccvaccaacuggucggggvnugaaac

SEQ ID NO: 30 - RNA, 30 nt, Artificial Sequence
  DR stem loop
  misc_feature (7)..(7): V is A or C or G
  misc_feature (22)..(22): H is A or C or U
  misc_feature (23)..(23): N is A or C or G or U
  misc_feature (24)..(24): N is A or C or G or U
aaccccvaccaacuggucggghnnugaaac

SEQ ID NO: 31 - RNA, 29 nt, Artificial Sequence
  DR stem loop
  misc_feature (6)..(6): V is A or C or G
accccvaccaacuggucgggguuugaaac

SEQ ID NO: 32 - RNA, 28 nt, Artificial Sequence
  DR stem loop
  misc_feature (5)..(5): V is A or C or G
ccccvaccaacuggucgggguuugaaac

SEQ ID NO: 33 - RNA, 27 nt, Artificial Sequence
  DR stem loop
  misc_feature (4)..(4): V is A or C or G
cccvaccaacuggucgggguuugaaac

SEQ ID NO: 34 - RNA, 31 nt, Artificial Sequence
  DR stem loop
  misc_feature (1)..(1): N is at least one additional A and/or C and/or G and/or U
  misc_feature (8)..(8): V is A or G or C
naaccccvaccaacuggucgggguuugaaac

SEQ ID NO: 35 - RNA, 30 nt, Artificial Sequence
  DR stem loop
  misc_feature (18)..(18): D is A or G or U
aaccccuaccaacuggudgggguuugaaac

SEQ ID NO: 36 - RNA, 30 nt, Artificial Sequence
  DR stem loop
  misc_feature (1)..(1): B is one of C or G or U
  misc_feature (18)..(18): D is A or G or U
baccccuaccaacuggudgggguuugaaac

SEQ ID NO: 37 - RNA, 30 nt, Artificial Sequence
  DR stem loop
  misc_feature (1)..(1): N is A or C or G or U
  misc_feature (2)..(2): B is C or G or U
  misc_feature (18)..(18): D is A or G or U
nbccccuaccaacuggudgggguuugaaac

SEQ ID NO: 38 - RNA, 30 nt, Artificial Sequence
  DR stem loop
  misc_feature (1)..(1): N is A or C or G or U
  misc_feature (2)..(2): N is A or C or G or U
  misc_feature (3)..(3): D is A or G or U
  misc_feature (18)..(18): D is A or G or U
nndcccuaccaacuggudgggguuugaaac

SEQ ID NO: 39 - RNA, 30 nt, Artificial Sequence
  DR stem loop
  misc_feature (18)..(18): D is A or G or U
  misc_feature (24)..(24): V is A or C or G
aaccccuaccaacuggudgggguvugaaac

SEQ ID NO: 40 - RNA, 30 nt, Artificial Sequence
  DR stem loop
  misc_feature (18)..(18): D is A or G or U
  misc_feature (23)..(23): D is A or C or G
  misc_feature (24)..(24): N is A or C or G or U
uuacccuaccaacuggudggggvnugaaac

SEQ ID NO: 41 - RNA, 30 nt, Artificial Sequence
  DR stem loop
  misc_feature (18)..(18): D is A or G or U
  misc_feature (22)..(22): H is A or C or U
  misc_feature (23)..(23): N is A or C or G or U
  misc_feature (24)..(24): N is A or C or G or U
aaccccuaccaacuggudggghnnugaaac

SEQ ID NO: 42 - RNA, 29 nt, Artificial Sequence
  DR stem loop
  misc_feature (17)..(17): D is A or G or U
accccuaccaacuggudgggguuugaaac

SEQ ID NO: 43 - DNA, 28 bp, Artificial Sequence
  DR stem loop
  misc_feature (16)..(16): D is A or G or U
ccccuaccaacuggudgggguuugaaac

SEQ ID NO: 44 - RNA, 27 nt, Artificial Sequence
  DR stem loop
  misc_feature (15)..(15): D is A or G or U
cccuaccaacuggudgggguuugaaac

SEQ ID NO: 45 - RNA, 31 nt, Artificial Sequence
  DR stem loop
  misc_feature (1)..(1): N is at least one additional A and/or C and/or G and/or U
  misc_feature (19)..(19): D is A or G or U
naaccccuaccaacuggudgggguuugaaac

SEQ ID NO: 46 - RNA, 30 nt, Artificial Sequence
  DR stem loop
aaccccuaccaacuggucgggguaugaaac

SEQ ID NO: 47 - protein, 7 aa, Artificial Sequence
  Flag tag
DYKDDDK

SEQ ID NO: 48 - protein, 1000 aa, Artificial Sequence
  Amino acid sequence of the Cas 13d variant
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

(19) United States
(12) Patent Application Publication
(10) Pub. No.: US 2023/0022311 A1
(43) Pub. Date: Jan. 26, 2023

(54) METHODS AND COMPOSITIONS INVOLVING CRISPR CLASS 2, TYPE VI GUIDES

(71) Applicants: NEW YORK GENOME CENTER, INC, New York, NY (US); NEW YORK UNIVERSITY, New York, NY (US)

(72) Inventors: Neville E. SANJANA, New York, NY (US); Hans-Hermann WESSELS, Brooklyn, NY (US); Alejandro MENDEZ-MANCILLA, New York, NY (US)

(21) Appl. No.: 17/756,459

(22) PCT Filed: Nov. 25, 2020
(86) PCT No.: PCT/US20/62379

§ 371 Date: May 25, 2022

Related U.S. Application Data
(60) Provisional application No. 62/940,575, filed on Nov. 26, 2019.
(60) Provisional application No. 62/952,922, filed on Dec. 23, 2019.
(60) Provisional application No. 63/060,757, filed on Aug. 4, 2020.

(51) Int. Cl. C12N 9/22

(52) U.S. Cl. CPC C12N 9/22; C12N 2310/20; C12N 2320/12

(57) ABSTRACT

A Class 2, Type VI clustered regularly interspaced short palindromic repeat (CRISPR) RNA (crRNA) which comprises a direct repeat (DR) stem loop sequence and a guide or spacer sequence, is provided characterized by a DR selected from those of Table 9. Also described is are methods for generating, selecting, characterizing and optimizing a clustered regularly interspaced short palindromic repeats (CRISPR) RNA (crRNA) for use in the CRISPR-Cas13d system described herein. Also provided is a screening method to identify crRNA particularly suited for use with specified targets. Further, the invention includes non-naturally occurring, synthesized or engineered crRNAs as described herein along with nucleic acid molecule, vectors, RNPs, cells, libraries, and compositions comprising the same, and uses thereof in treating a disease or in functionally screening a gene. A method for blocking an RNA target without degradation and a method for modification of multiple RNA targets using the same CRISPR effector protein are also disclosed.

### CROSS-REFERENCE TO RELATED APPLICATIONS

This application claims the benefit of the priority of U.S. Provisional Patent Application No. 62/940,575, filed Nov. 26, 2019; U.S. Provisional Patent Application No. 62/952,922, filed Dec. 23, 2019; and U.S. Provisional Patent Application No. 63/060,757, filed Aug. 4, 2020, which applications are incorporated herein by reference.

### STATEMENT REGARDING FEDERALLY SPONSORED RESEARCH OR DEVELOPMENT

This invention was made with government support under grant numbers R00HG008171, DP2HG010099, and R01CA218668 awarded by the National Institutes of Health and grant number D18AP00053 awarded by the Defense Advanced Research Projects Agency. The government has certain rights in the invention.

### INCORPORATION-BY REFERENCE OF MATERIAL SUBMITTED IN ELECTRONIC FORM

Applicant hereby incorporates by reference the Sequence Listing material filed in electronic form herewith. This file is labeled NYG-LIPP120PCT_ST25.txt”, was created on 25 Nov. 2020, and is 23 KB in size.

### BACKGROUND OF THE INVENTION

Class 2, Type VI clustered regularly interspaced short palindromic repeats (CRISPR) enzymes (for example, Cas13 proteins) have recently been identified as programmable RNA-guided, RNA-directed Cas proteins with nuclease activity that allow for target gene knock-down without altering the genome.

To date, three different Cas13 effector proteins (PguCas13b, PspCas13b, RfxCas13d) have been reported to show high RNA knock-down efficacy with minimal off-target activity 9,11 In addition to target RNA knock-down¹²⁻¹⁷, Cas13 proteins have been used to enable viral RNA-detection systems^{18,19}, site-directed RNA-editing²⁰, demethylation of m⁶A-modified transcripts ²¹, RNA live-imaging and modulation of splice site choice as well as cleavage and polyadenylation site usage 22-24

Cas13 proteins are guided to their target RNAs by a single CRISPR RNA (crRNA) composed of a direct repeat (DR) stem loop and a spacer sequence (guide RNA) that mediates target recognition by RNA-RNA hybridization. Although Cas13 enzymes exert some non-specific collateral nuclease activity upon activation^{15,16,18,25,26} they have greatly reduced off-target target activity in cultured cells compared to RNA interference Previous studies have shown that Cas13 guide RNAs have minimal Protospacer Flanking Sequence (PFS) constraints in mammalian cells^{12,15,20,27} and that RNA target sites should be preferentially accessible for Cas13 binding 12,13,15.

Compared to DNA-targeting CRISPR nucleases like Cas9 or Cas12a/Cpf1, little is known about targeting preferences of RNA-targeting Class 2, Type VI CRISPR enzymes like Cas13d. Despite the notion that Cas13d enzymes have limited protospacer adjacent sequence restrictions and that mRNA target sites should be relatively accessible, there is no guidance for the spacer RNA (guide) design for these novel enzymes.

Beyond these basic parameters, we currently lack information about optimal Cas13 crRNA designs for high target RNA knock-down efficacy. A continuing need in the art exists for new and effective tools and methods for screening, designing, optimizing, ranking, selecting and using CRISPR Cas crRNAs with high specificity and efficiency but low off-target activity.

### SUMMARY OF THE INVENTION

In one aspect, a non-naturally occurring, synthesized or engineered crRNA Class 2, Type IV clustered regularly interspaced short palindromic repeat (CRISPR) RNA (crRNA) is provided which comprises a direct repeat (DR) stem loop sequence and a guide or spacer sequence, said DR selected from one or more of the DR sequences or a modification thereof of Table 9, SEQ ID Nos; 1-46, wherein R represent A or G; Y represents C or T(or U); S represents G or C; W represents A or T(or U); K represents G or T(or U); M represents A or C; B represents C or G or T(or U); D represents A or G or T(or U); H represents A or C or T(or U); V represents A or C or G; N represents any base; and - represents a nucleotide gap.

In another aspect, a nucleic acid molecule is provided that comprises the crRNA identified above. In some embodiments, the crRNA is capable of forming a complex with a Class 2, Type VI effector protein, and directing the complex to bind to the target RNA to cleave or block the target RNA. In some embodiments, the Class 2, Type VI effector protein is a CRISPR-associated protein 13d (Cas13d). In some embodiments, the nucleic acid molecule is a vector or plasmid. In some embodiments, the vector is a viral vector.

In another aspect, a nucleic acid molecule is provided in which the crRNA comprise a DR sequence of Table 9 and guide sequences which mismatch the target and allow the Class 2, Type VI effector protein to bind the target, but not elicit target degradation.

In another aspect, a ribonucleoprotein (RNP) complex comprises a Class 2, Type VI effector protein and a crRNA as described above.

In another aspect, a composition comprises a crRNA or RNP as described herein, or a nucleic acid molecule as described herein in a pharmaceutically acceptable carrier. In certain embodiments, the carrier is a nanoparticle, a lipid complex, a polymer, a quantum dot, a carbon nanotube, a magnetic nanoparticle, or a gold nanoparticle.

Still other aspects include a cell comprising any of the nucleic acid molecules, crRNA, RNP or compositions described herein, or a library comprising a plurality of crRNAs, nucleic acid molecules or viral vectors described herein, wherein each of the crRNA is capable of directing a Cas13d or a variant thereof to a different target RNA or a different region of one target RNA. Other aspects further include a pharmaceutical composition comprising a crRNA, nucleic acid molecule, RNP, composition, cell or library as described herein.

In still another aspect, a method of treating a disease associated with an abnormal RNA or misregulation of an RNA transcript, comprises administering to a subject in need thereof the crRNA, nucleic acid molecule, RNP and/or pharmaceutical compositions described herein. In yet a further aspect, a method of improving the efficiency of, or stabilizing the targeting of a Class 2, Type VI clustered CRISPR RNA (crRNA) comprising a direct repeat (DR) stem loop and a guide or spacer sequence is provided. An exemplary method entails replacing the DR stem loop sequence of a less efficient crRNA with a DR sequence selected from one or more of the DR sequences of SEQ ID Nos: 1 to 46, or a modification thereof.

In still a further embodiment a method is provided for screening or predicting on-target activity of a clustered regularly interspaced short palindromic repeats (CRISPR) RNA (crRNA), which crRNA is capable of forming a complex with an RNA-targeting CRISPR-associated protein or a variant thereof and directing the complex to the target RNA. The method comprises the steps of (a) characterizing a plurality of crRNAs and their corresponding target by features comprising the presence of both a seed region located between guide RNA nucleotide bases 15 to 21 relative to the guide RNA 5′ end, characterized by a stabilizing, enriched sequence of G and C bases and an accessible target region characterized by an enriched sequence of A and U, surrounding the seed region on the 5′ end, 3′ end or both the 5′ and 3′ ends; (b) assessing on-target activity of each of the crRNAs of (a); (c) applying a machine learning model or deep learning model using the characterization of (a) and the on-target activity of (b). Input of the model comprises characterization(s) of said seed region and target regions of each crRNA and its corresponding target RNA, and output of the model is an on-target score of the crRNA. A higher score indicates a ranked on-target activity. The method also includes (d) applying the model constructed in step (c) to a first crRNA and generating an on-target score of the first crRNA. In one embodiment, the crRNA are characterized by the DR sequences recited in Table 9.

In another aspect, a method of blocking RNA regulatory elements without degradation of the target nucleic acid is provided. The method includes the step of administering cRNAs to a cell expressing an RNA-targeting CRISPR-associated protein or to a subject. The crRNAs are capable of forming a complex with the RNA-targeting CRISPR-associated protein or a variant thereof and directing the complex to the target RNA. The crRNAs comprise a DR sequence and a guide or spacer sequences, said guide or spacer sequences forming extended mismatches to the target site in the seed region.

In another aspect, a method is provided for generating and selecting a clustered regularly interspaced short palindromic repeats (CRISPR) RNA (crRNA) composed of a direct repeat (DR) stem loop and a guide. The selected crRNA is capable of forming a complex with a CRISPR-associated protein 13d (Cas13d) or a variant thereof and directing the complex to a target RNA. The method comprises randomly designating a potential hybridization region in the target RNA, designing a guide which is capable of hybridizing to the hybridization region, designing a crRNA sequence comprising the guide and a DR stem loop accordingly, and ranking each crRNA based on features of the crRNA and its corresponding target RNA. In one embodiment, the features comprise one or more of those listed in Tables 2 and 4-7 and FIGS. 6 and 13. In certain embodiments, the crRNA(s) with the highest ranking is selected for directing the Cas13d-crRNA complex to the target RNA. In certain embodiments, one or more other features and/or features within certain ranges are utilized in ranking the crRNAs. Also provided is a crRNA selected using the disclosed method.

In one embodiment, a crRNA or its corresponding target RNA having a feature within the identified range of a positively-correlated feature ranks higher than those falling out of the range. Additionally, or alternatively, a crRNA or its corresponding target RNA having a feature out of the identified range of a negatively-correlated feature ranks higher than those falling within the range. The ranges may include one or more of those listed in Tables 2, 4,5 and 7.

In another aspect, provided is a non-naturally occurring, synthesized (chemically or recombinantly) or engineered crRNA composed of a direct repeat (DR) stem loop and a guide capable of hybridizing to a hybridization region of a target RNA. The crRNA is capable of forming a complex with a CRISPR-associated protein 13d (Cas13d) or a variant thereof and directing the complex to the target RNA. In a further embodiment the crRNA or the corresponding target RNA comprises a feature which falls within a certain range of one or more of the positively-correlated features and out of a certain range of one or more of the negatively-correlated features as illustrated in Tables 2, 4 and 5.

Additionally, provided are nucleic acid molecules, vectors, and compositions comprising a nucleic acid sequence of a crRNA as disclosed or a nucleic acid sequence encoding the crRNA, along with a library comprising a plurality of the crRNAs, nucleic acid molecules, or vectors. Uses of the disclosed components or compositions are further provided, for example, treatment of a disease associated with an abnormal RNA, or genome-wide screening for functional RNAs.

Still other aspects and advantages of these compositions and methods are described further in the following detailed description of the preferred embodiments thereof.

### BRIEF DESCRIPTION OF THE DRAWINGS

FIGS. 1a to 1h. CRISPR Type VI-D RfxCas13d GFP knock-down pooled tiling screen. (1a) The GFP-targeting library contained 400 guide RNAs that were perfectly matched, 100 guide RNAs with a single mismatch at each of the 27 guide positions (n=2,700 guides), 30 guide RNAs with 100 random double mismatches (n=3,000 guides), and 17 guides with consecutive double (n=442 guides) and triple (n=425 guides) mismatches. CrRNAs are lentivirally transduced into double-transgenic TetO-RfxCas13d and GFPd2PEST HEK293 cells. After selection, cells are sorted by GFP intensities into 4 bins. (1b, 1c) Log₂ fold-change (log 2FC) enrichment scores of guide RNAs comparing guide RNA counts of the lowest fluorescence (Bin 1) to the input (unsorted) cell population. Scores are demarcated by the type of designed crRNAs as given by the list in a. (1B) All crRNAs. (1c) A single perfect match (PM) crRNA and corresponding derivative crRNAs with mismatches. Guide log 2FC enrichments are calculated relative to the perfect match reference guide (Δlog₂FC). Black lines denote medians. (1d) Distribution of perfect match guide RNAs along the GFP mRNA and their log 2FC enrichment. Guide RNAs are separated into targeting efficiency quartiles Q1-Q4 with Q4 containing guides with the best knock-down efficiency. (1e) GFP knock-down validation for 6 guides (3 with high efficacy and 3 with low efficacy) highlighted in d. (n=3 transfection replicates; Veh=vehicle transfection, NT=non-targeting crRNAs). Significance from a one-tailed t-test. (f) Relative targeting efficacy (Δlog₂FC) of guides with single nucleotide mismatches (SM) at the indicated position relative to their cognate perfect match guides. Significance: * p<0.05, ** p<0.01, *** p<0.001 from a two-tailed t-test. (1g) (top) Change in targeting efficacy by guide RNA nucleotide identity or mismatch type. (bottom) Change in targeting efficacy for SM, CD, or consecutive triple mismatch (CT) by position. (1h) Validation of RfxCas13d seed region. (left) Individual perfect match and mismatch guides relative to GFP target mRNA. (right) Percent GFP negative cells after co-transfection of specific GFP-targeting crRNAs normalized to the non-targeting control. Veh=vehicle transfection, NT=non-targeting crRNA, PM=perfect match guide RNA.

FIGS. 2a to 2f. RfxCas13d on-target guide RNA prediction model. (2a) Correlation of predictions from a Random Forest (RF) regression model (either with all features or a minimal set of the most predictive features) and a support vector machine with L1 regression to held-out screen data. (2b) Validation of on-target model testing 3 high-scoring and 3 low-scoring guide RNAs via targeting of cell-surface proteins and antibody labeling to measure target knock-down by FACS. Relative knock-down indicates the percent reduction (relative to non-targeting guide RNAs) in the mean fluorescence intensity. (n=3 transfection replicates; one-tailed t-test). (2c) Validation of on-target model assaying 3 high-scoring and 3 low-scoring guide RNAs per gene in a gene essentiality screen in HEK293 cells with growth dropout phenotype testing 10 essential genes and 10 control genes. Each point represents one guide as a mean of three replicate experiments. The y-axis depicts the log₂ fold-change (FC) of the guide RNA at the indicated time point relative to the Day 0 sample. One-sided KS-test comparing high-scoring and low-scoring guides, *** p=2×10⁻⁵, **** p=2×10⁻⁶. (2d) A375 essentiality screen with growth dropout phenotype assaying 20 high-scoring and 20 low-scoring guide RNAs per gene. One-sided KS-test comparing high-scoring or low-scoring guides to the distribution of non-targeting guides. * p=0.043, ** p=0.0095, **** p<1×10⁻⁴⁴. (2e) Gene ranking for essentiality based on the robust rank aggregation (RRA) p-value across replicates for all 20 high scoring guides. Blue dots denote essential genes from a prior RNAi screen⁹. (2f) Spearman rank correlation of Cas13d gene depletion (as in e) with prior CRISPR-Cas9 and RNAi screens in A375 cells. Analysis includes genes represented in all libraries (n=35 essential genes and n=15 control genes). (RNAi screen: A375 DEMETER2 v5 score⁹, Cas9 screen: A375 STARS score⁴).

FIGS. 3a to 3f. Improvement of RfxCas13d on-target guide RNA prediction model with tiling screens over endogenous transcripts. (3a-3c) Distribution of perfect match guide RNAs along the coding region (CDS) of CD46, CD55 and CD71 mRNA and their log₂ fold-change (FC) enrichments. Positive FC values indicate better transcript knock-down. Guide RNAs are separated into targeting efficiency quartiles Q1-Q4 per gene with Q4 containing guides with the best knock-down efficiency. Numbered bars below indicate exons. (3d) Correlation of predictions from the RFₘᵢₙᵢₘₐₗ (=RF_{GFP}) model and the updated RF_{combined} regression model to held-out screen data using bootstrapping across all four tiling screens. (3e) Comparison of predicted and measured log 2FC quartiles across the 10-fold model cross-validation. Quartile definition as in 3a-3c. (3f) Spearman rank correlation between observed guide RNA depletion (=target knock-down) and the predicted guide score for the indicated Cas13d essentiality screens and indicated on-target models (see FIGS. 2c-2d and FIG. 11).

FIGS. 4a to 4d Processing of crRNA count data from the GFP pooled screen (4a) Gating strategy for GFP tiling screen (see Example 2). (4b) Log₂-transformed crRNA counts for three replicate screen input and bin 1 libraries showing flagged technical and non-reproducible outlier counts with high residuals deviating from a linear regression model. Flagged crRNA counts of individual samples were not considered during crRNA count processing (see outlier removal in Examples). (4c) Spearman (rₛ) and Pearson (rₚ) correlations coefficients of crRNA counts across all 15 libraries during crRNA count processing. While Spearman correlations on the raw count data and terminally processed data looked comparable, outlier-removal greatly increase Pearson correlations between replicates and emphasized distance between different bins. (4d) Principal components (PC) 1-3 corresponding to crRNA processing steps in (4c).

FIGS. 5a to 5d. crRNA structure affects crRNA targeting efficacy. (5a) Scatterplot showing the crRNA log₂FC versus the predicted crRNA secondary structure minimum free energy (MFE). The Pearson correlation coefficient (rₚ) is nearly unchanged (rₚ=0.44) when MFEs of G-Quadruplex-forming crRNAs are ignored. (5b) (left) DR modification tested in (5c). Key: green=paired nucleotide, red=unpaired nucleotide in bulge or loop, blue=unpaired nucleotide preceding guide RNA, underlined bases have been changed relative to the wild-type reference. Deleted bases are not shown. (right) Predicted DR secondary structure of two selected DR modifications using RNAfold. (5c) Target knock-down comparison varying the DR sequence using GFP-targeting guide G3 used in FIG. 4c. RfxCas13d-NLS expressing cells were co-transfected with plasmids delivering the crRNA and with a GFP-encoding plasmid. Shown is the percentage of mean fluorescence intensity reduction of cells transfected with a GFP-targeting guide relative to a non-targeting guide as a mean of three replicate experiments. Error bars indicate standard error of the mean. (5d) Target knock-down comparison comparing the wildtype DR (WT) and stem disruption 1 DR (as in b) across 6 GFP-targeting guide RNAs with either low or high knock-down relative to a non-targeting guide control (Guides as used in FIG. 1e). (n=3 transfection replicates). One-tailed t-test in c and d with significance levels: * p<0.05, ** p<0.01, *** p<0.001)

FIGS. 6a to 6f. Comparison of machine learning approaches and feature selection for Cas13d guide RNA ‘on-target’ model. (6a) Model performance evaluation of linear models and learning approaches using features as in Table 6. We compared the ability of machine leaning regression approaches to predict target knock-down of held-out data using bootstrapping. The data of all perfectly matching guides (n=399) from the GFP tiling screen and features was randomly split into 70% training data and 30% held-out testing data for 1000 random non-redundant splits. The prediction accuracy (comparing predicted scores to the known log 2FC) is computed using the Spearman correlation (rₛ) to the held-out data. The number inside the box is the median of 1000 bootstrap samplings. The models are ranked by their median performance. (NT-context=linear combination of all nucleotide context values. NT-context+=NT-context plus crRNA MFE). (6b) Boxplots showing the percent increase in mean-squared error (% IncMSE) of features for the top-scoring Random Forest model in (6a). (6c) Feature selection of selected machine learning approaches. Shown is the frequency that a particular feature was selected for the best model across 1000 iterations from (6a). (6d) % IncMSE of features for the top-scoring Random Forest model using a minimal set of selected features, corresponding to the RFₘᵢₙᵢₘₐₗ (=RF_{GFP}) model in FIG. 2a. (6e) Comparison of predicted and measured fold-change quartiles across the 10-fold RF_{GFP} model cross-validation. (6f). Prediction of standardized guide scores by the RF_{GFP} model. Shown are the median predicted guides scores for the top/bottom guide RNAs (N=2, 4, 8, 16, 32) ranked by the known guide RNA efficacy across the 10-fold cross-validation. Error bars indicate the standard error of mean. Grey shading indicates the null distribution for the median guides scores of randomly selected crRNAs across 1000 samplings for each N.

FIGS. 7a to 7d. RF_{GFP} on-target model validation. (7a). Scatterplot showing the log 2FC depletion scores provided by the DEMETER2 v5 repository of HEK-TE cells (y-axis) and the median log 2FC across 712 cell lines present (x-axis). Genes targeted in the HEK293 cell essentiality screen are colored by gene type (essential, control). (7b) Guide RNA depletion (log₂FC) in pooled HEK293 cell essentiality screen at Day 27 relative to input samples. Shown are the individual guide enrichments colored predicted guide scoring class (high vs. low) of three replicate experiments. Genes are ranked by the mean depletion of the predicted high-scoring guides. (inset) Distribution of the 30 most depleted guide RNAs across four targeting classes (EG H: High-scoring guide RNAs targeting essential genes; EG L: Low-scoring guide RNAs targeting essential genes; Ctrl H: High-scoring guide RNAs targeting non-essential genes; Ctrl L: Low-scoring guide RNAs targeting non-essential genes). (7c) Selected target genes for A375 cell essentiality screen similar to a. (7d). Boxplot summarizing the log₂FC depletion of predicted low- and high-scoring guide RNAs for the 35 selected essential genes comparing Day 14 to input samples. For b and d we used a one-sided t-test of log₂FC values of high-scoring guide RNAs versus low-scoring guide RNAs per gene. Significance levels: * p<0.05, ** p<0.01, *** p<0.001.

FIG. 8. Target site accessibility. Heatmap depicting the Pearson correlation coefficient (rₚ) between the local target site accessibility (=log₁₀ (unpaired probability)) and the observed log₂FC relative to guide RNA match positions. We performed a grid-search correlating the observed guide RNA efficacies with the login-transformed unpaired probability in a window (w) of 1 nt up to 50 nt at every point 20 nt 5′ of the target site to 20 nt 3′ of the target site.

FIG. 9. Overview of guide RNA and target RNA feature windows. For guide RNA features nucleotide 1 defines the guide start site (GSS) being the most 5′ guide RNA base matching the target RNA. Nucleotide 2 relative to GSS is the subsequent base (moving in the 5′ to 3′ direction) in the guide RNA and so on. For target RNA features, we denote the target nucleotide opposite to the GSS as nucleotide 0. Moving in 5′ to 3′ direction target RNA nucleotide −1 is upstream to the GSS and pairs with guide nucleotide 2, while target RNA nucleotide +1 is downstream of the target site and so on.

FIG. 10. Overview of guide RNA and target RNA feature windows. For guide RNA features nucleotide 1 defines the guide start site (GSS) being the most 5′ guide RNA base matching the target RNA. Nucleotide 2 relative to GSS is the subsequent base (moving in the 5′ to 3′ direction) in the guide RNA and so on. For target RNA features, we denote the target nucleotide opposite to the GSS as nucleotide 0. Moving in 5′ to 3′ direction target RNA nucleotide −1 is upstream to the GSS and pairs with guide nucleotide 2, while target RNA nucleotide +1 is downstream of the target site and so on. Selected features with either positive or negative correlation are denoted with the subscript ‘max’ or ‘min’, respectively, in Table 7.

FIG. 11. Distribution of processed guide RNAs counts in Input and Sort Bin 1 (bin with strongest GFP knock-down) samples for all perfect match guide RNAs in the GFP screen (n=399). Shown are the mean counts across three replicates. The data is binned into guide RNA log₂FC enrichment quintiles. 80-100% represents the 20% most enriched guide RNAs, and 0-20% represents the 20% most depleted guide RNAs.

FIG. 12. Nucleotide probability windows selected for the initial RF_{GFP} on target model. Each window was defined based on the correlation of nucleotide probability with the observed guide RNA enrichments (log₂FC). All features for the initial RF_{GFP} on target model can be found in Table 6.

FIG. 13. Feature Importance of the RF_{combined} on target model.

FIG. 14a-14c. Optimal CRISPR-Cas13d gRNAs to target common human pathogenic RNA viruses. (14a) World map of analyzed SARS-CoV-2 isolates (data from GISAID, Apr. 17, 2020). (14b) Guide RNA design for each SARS-CoV-2 gene. Top panel: SARS-CoV-2 gene annotations. Middle panel: Percent of SARS-CoV-2 genomes targeted by each NY1 reference gRNA. Bottom panel: Fraction of gRNAs in Q4 per gene (pie) and total number of Q4 gRNAs per gene that targets at least 99% of the total genomes (bar). (14c) Predicted minimum number of Q4 gRNAs to target all SARS-CoV-2, MERS-CoV, H1N1, and HIV-1 genomes analyzed (n=7630, 522, 4237 and 5557 viral genomes, respectively).

FIG. 15. Transcript length for mRNAs and ncRNAs across species. Dotted line indicates the minimal input length requirements (>80 bp) for Cas13d design software. Transcript lengths were derived from corresponding gene annotation reference sequences.

FIG. 16. Q4 gRNAs targeting coding SARS-CoV-2 regions verses noncoding SARSCoV-2 regions. Classification of coding and noncoding regions is from the NCBI annotation of the SARSCoV-2 reference strain.

FIG. 17a-17e illustrates the mismatching concept disclosed herein. FIG. 17a is a general overview of this approach with the example of the V600E mutation in the BRAF gene. FIGS. 17b-17e show different visualization of SNV specific targeting for four genes with predicted malignant outcome. FIG. 17b describes the proportion of reference versus SNV base upon Cas13d targeting detected by sequencing. FIG. 17c quantifies the observed changes as a log₂ fold change relative to the wild type state for the SNV base (left) or reference base (right). The SNV base changes with a log₂ fold change relative to the abundance in the wild type state specifically when the SNV carrying transcript is targeted (gRNA mut; red dot). FIG. 17d shows the same data but quantifies the delta/difference in the base probability. FIG. 17e shows the example of the IMMT gene data and how the observed base probabilities change presented as an average sequence motif.

### DETAILED DESCRIPTION

Described herein is a method of screening and selecting a clustered regularly interspaced short palindromic repeats (CRISPR) RNA (crRNA) suitable for forming a complex with a CRISPR-associated protein 13d (Cas13d) or a variant thereof and directing the complex to a target RNA. The method comprising randomly designating a potential hybridization region in the target RNA, designing a guide which is capable of hybridizing to the hybridization region, designing a crRNA comprising the guide and a DR stem loop accordingly, and ranking each crRNA based on crRNA-specific features as well as corresponding target RNA features. Using the method, crRNAs with high knock-down efficacy are selected. In one embodiment, the method is in silico.

Also provided are a non-naturally occurring, synthesized or engineered crRNA selected and generated according to the method, along with a vector, a nucleic acid molecule, a library of vectors or nucleic acid molecules, and a composition comprising the crRNA. Methods and uses of the disclosed crRNA(s), vector(s), nucleic acid molecule(s), library(ies) and composition(s) are also provided, for example, in the treatment of a disease associated with an abnormal RNA, in a genome-wide screening of functional RNA, and detecting, knocking-down, editing, or modifying a target RNA. More details are described below.

The methods and compositions described herein provide optimal Cas13 crRNA designs for high target RNA knock-down efficacy. Additionally, such methods and compositions address, among other issues, how mismatches relative to the target site affect Cas13d activity and leverage this aspect for the development of novel biotechnologies.

### A. Components

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

As used herein, “crRNA” is an abbreviation of clustered regularly interspaced short palindromic repeats (CRISPR) RNA, which is a nucleic acid molecule composed of a direct repeat (DR) stem loop sequence and a guide sequence. The terms “guide RNA” “guide” or “guide sequence” refer to a nucleic acid sequence which can hybridize to a sequence (hybridization region or target region) of a target RNA. The guide is capable of complexing with Cas13d protein and providing targeting specificity and binding ability for Cas13d. In one embodiment, the guide RNA is about 20 nucleotides (nt) to about 33 nt. In a further embodiment, the guide RNA is about 20 nt, about 21 nt, about 22 nt, about 23 nt, about 24 nt, about 25, nt, about 26, nt, about 27 nt, about 28 nt, about 29 nt, about 30 nt, about 31 nt, about 32 nt, or about 33 nt. In one embodiment, the guide RNA is about 27 nt. Existence of a sequence in a target RNA similar to a protospacer or protospacer adjacent motif (PAM) was not found in the CRISPR-Cas13d system as further detailed in the Detailed Description and Examples.

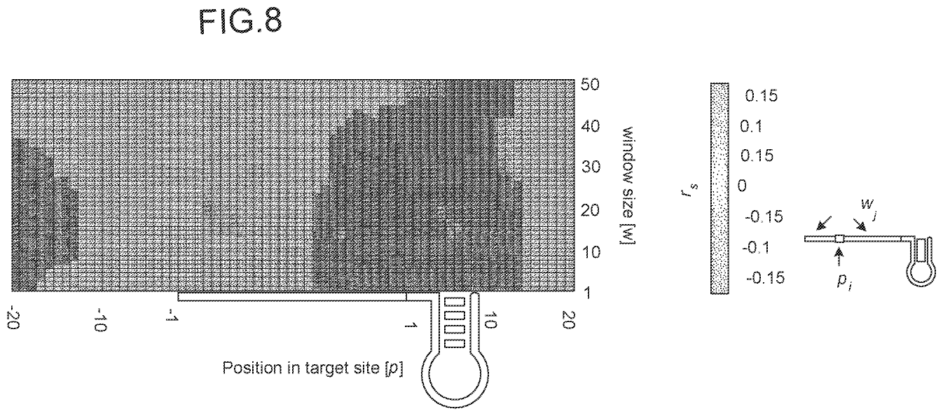

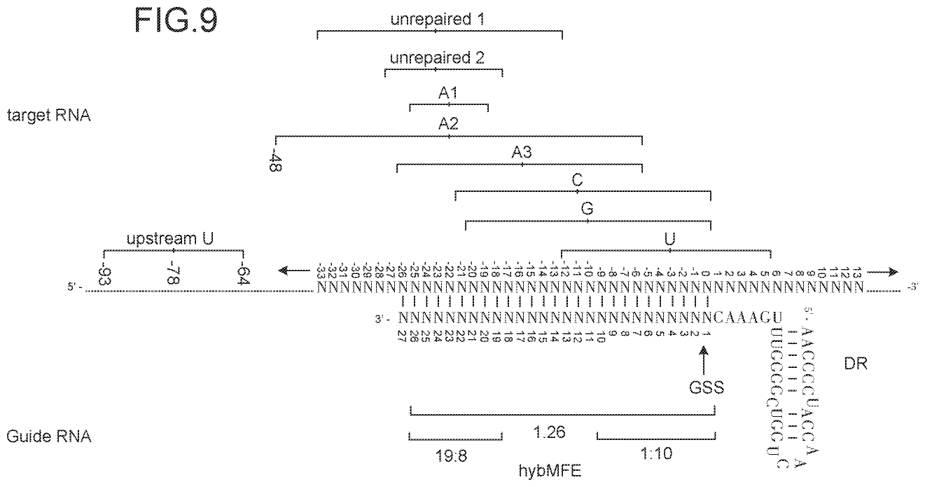

As used herein, nucleotide residues in a crRNA or a portion of it (for example, a guide, or a stem loop), unless specified, are numbered as illustrated in FIG. 9, 10, or 12. In one embodiment, the numbering is further illustrated in Example 5. In one embodiment, the numbering is based on a numbering from 5′ end of the crRNA to 3′ end recognizing the guide match start as nt 1. The guide match start is the first nucleotide residue (nt) from the 5′ end of the crRNA which is capable of matching to a nt of a target RNA. The nt numbering at the 3′ side of the guide match start is a positive integer positively correlated to its distance to the guide match start, while the nt numbering at the 5′ side of the guide match start is a negative integer whose absolute value is positively correlated to its distance to the guide match start. One exception is the last nt of the DR stem loop contiguously proceeding the first nt of the guide is numbered as nt 0. As one of skill in the art would appreciate, whenever an order of a nt is implying, for example, via using the terms “first” “last” “proceeding” or similar, the order is counted from the 5′ end to the 3′ end. Further, with respect to a target RNA or a portion of it (for example, a hybridization region of the target RNA), the nt numbering is from 5′ end of the target RNA to its 3′ end recognizing the nt which is capable of matching to the guide match start as nt 0. The nt numbering at the 3′ side of the nt matching to the guide match start is a positive integer positively correlated to its distance to the guide match start, while the nt numbering at the 5′ side of the nt matching to the guide match start is a negative integer whose absolute value is positively correlated to its distance to the guide match start. An illustration can be found in FIG. 8. Based on the definitions herein, one of skill in the art would understand that nt x of a crRNA matches to nt −(x−1) of a corresponding target RNA, wherein x is a positive integer. In one embodiment, for guide RNA features nucleotide 1 defines the guide start site (GSS) being the most 5′ guide RNA base matching the target RNA. Nucleotide 2 relative to GSS is the subsequent base (moving in the 5′ to 3′ direction) in the guide RNA and so on. For target RNA features, we denote the target nucleotide opposite to the GSS as nucleotide 0. Moving in 5′ to 3′ direction target RNA nucleotide −1 is upstream to the GSS and pairs with guide nucleotide 2, while target RNA nucleotide +1 is downstream of the target site and so on. In a further embodiment, a range of nt is also illustrated as nucleotide position p over the distance d to the position p+d with its cognate sequence. In another embodiment, a nt range is noted as (nt x: y) indicating nt x to nt y, wherein x and y is an integer which may be positive, negative or zero.

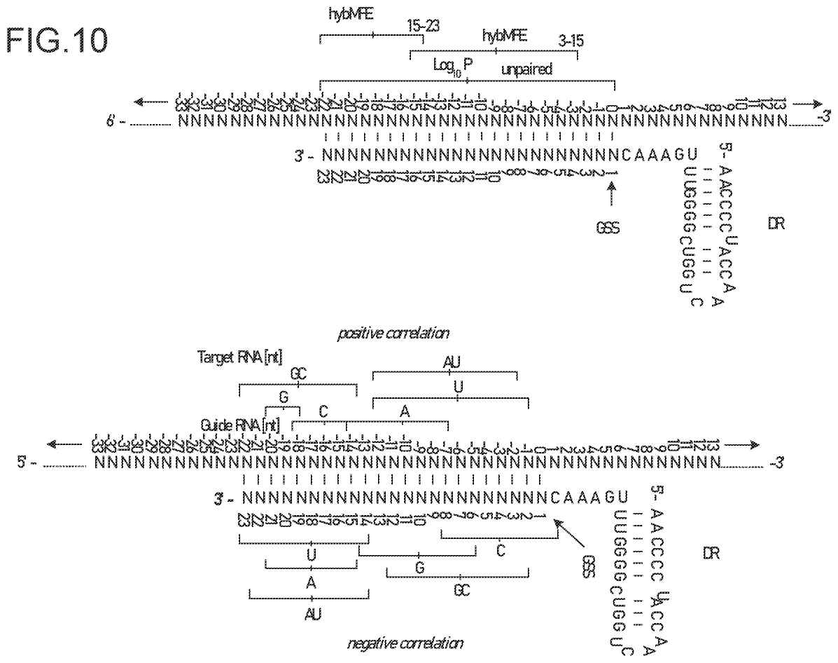

In one embodiment, features with either positive or negative correlation are denoted with the subscript ‘max’ or ‘min’, respectively, in Table 7 as well as in FIG. 10. In one embodiment, except G-quadruplex, a feature without “max” or “min” there in is a positively correlated feature. In one embodiment, presence of G-quadruplex is a negatively correlated feature, i.e., absence of G-quadruplex is a positively correlated feature. A suitable feature is also obvious to one of skill in the art in view of the Examples provided herein.

In the embodiments relating to a nucleic acid molecule or a vector or uses thereof, a nucleic acid molecule encoding a crRNA may be in operative association with an RNA pol III promoter. A RNA pol III promoter is a promoter that is sufficient to direct accurate initiation of transcription by the RNA polymerase III machinery, wherein the RNA polymerase III (RNAP III and Pol III) is a RNA polymerase transcribing DNA to synthesize ribosomal 5S ribosomal RNA (rRNA), transfer RNA (tRNA), crRNA, and other small RNAs. A variety of Polymerase III promoters which can be used are publicly or commercially available, for example the U6 promoter, the promoter fragments derived from H1 RNA genes or U6 snRNA genes of human or mouse origin or from any other species. In addition, pol III promoters can be modified/engineered to incorporate other desirable properties such as the ability to be induced by small chemical molecules, either ubiquitously or in a tissue-specific manner. For example, in one embodiment the promoter may be activated by tetracycline. In another embodiment, the promoter may be activated by IPTG (lacI system). See, U.S. Pat. Nos. 5,902,880A and 7,195,916B2. In another embodiment, a Pol III promoter from various species might be utilized, such as human, mouse or rat.

As used herein a “target RNA” refers to an RNA molecule or a nucleic acid molecule to which a guide sequence is designed to target, e.g. have complementarity, where hybridization between a target RNA and a guide promotes the formation of a CRISPR-Cas13d complex. In one embodiment, the target RNA comprises at least 20 nt (or at least 23 nt, or at least 87 nt, or at least 100 nt) RNA residues or a modification thereof. In a further embodiment, the target RNA comprises at least 20 nt contiguous RNA residues or a modification thereof. The region of a target RNA which is capable of hybridizing to a guide of a crRNA is referred to herein as a potential hybridization region. Such target RNA, a hybridization region therein, a crRNA which the hybridization region of the target RNA may hybridize to, and a guide of the crRNA are corresponding to each other.

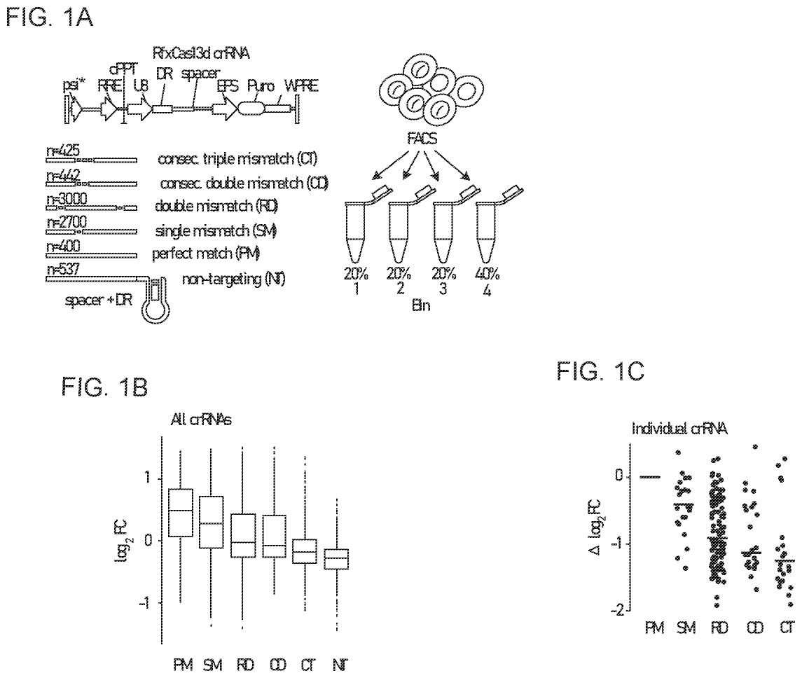

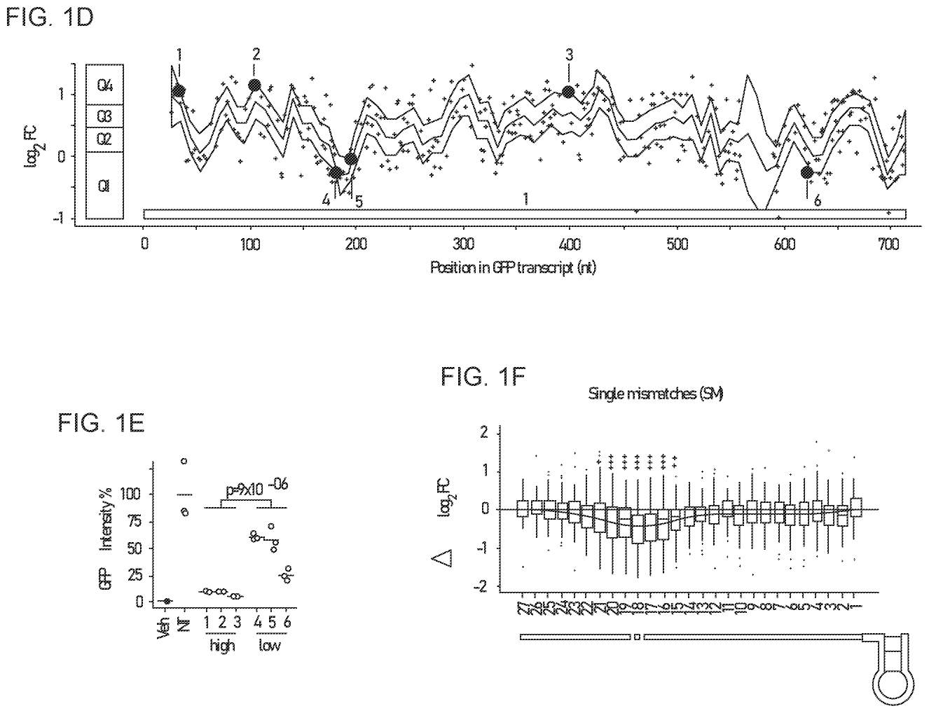

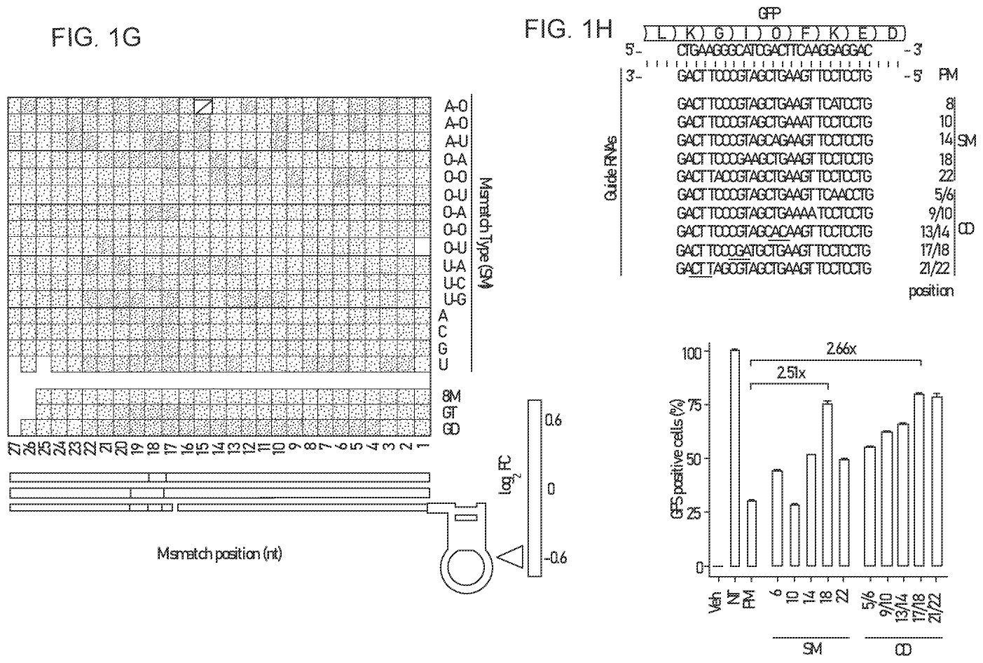

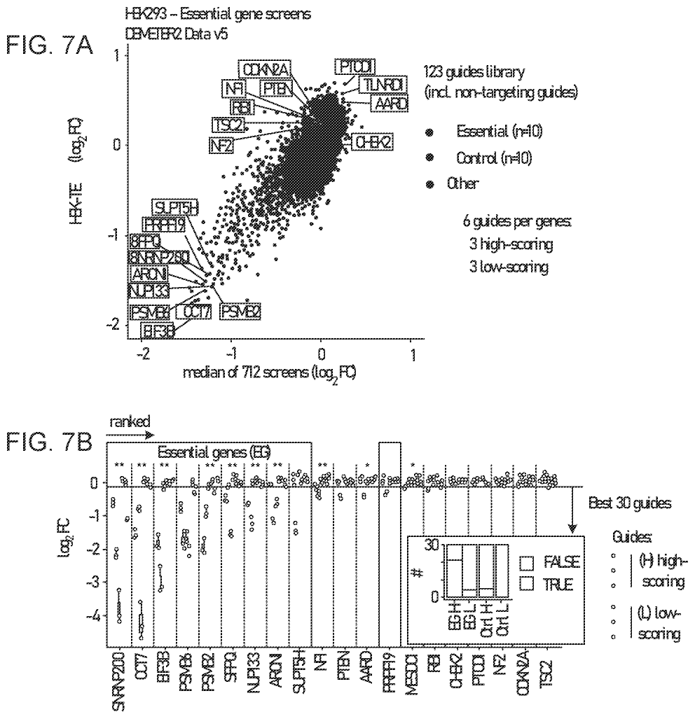

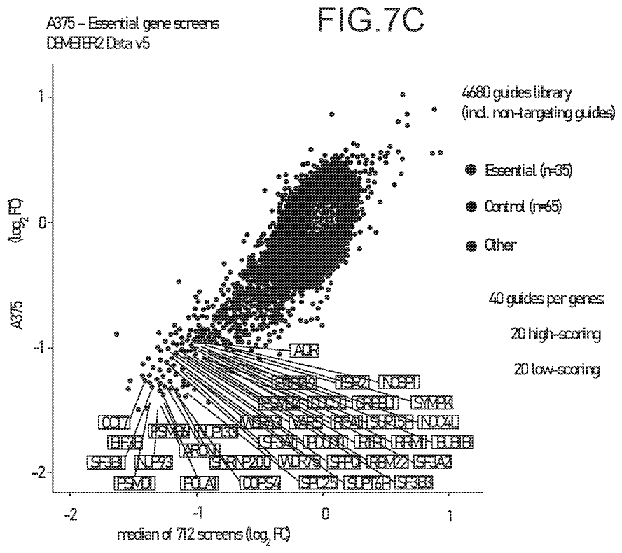

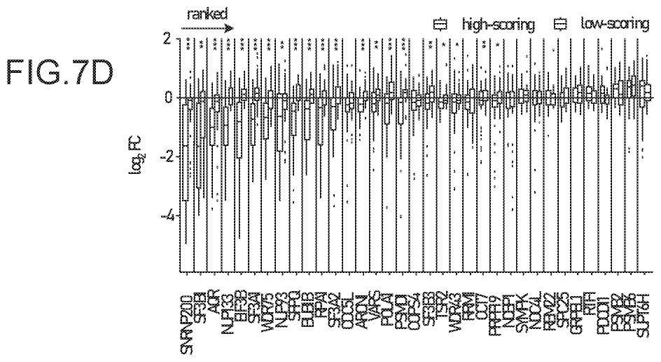

As used herein, the term “seed” region or any other grammatical variation thereof means a critical region of the target sequence of Class 2, Type VI enzymes (e.g., Cas13d) that must be strictly complementary to the CRISPR RNA guide to ensure knock-down efficacy. Mismatches between the target and CRISPR RNA guide sequence can contribute to off-target activity. In one embodiment, the critical Cas13d seed region is defined as the region located between guide RNA nucleotides 15 to 21. In another embodiment, the seed region is defined as the region located between guide RNA nucleotides 15 to 21, with its center at nucleotide 18 relative to the guide RNA 5′ end. Within the seed region, single mismatches lead to diminished guide enrichment, while mismatches outside the seed region were better tolerated (see FIG. 1f). The critical region was present irrespective of the mismatch identity (FIG. 1g). Similarly, consecutive double and triple mismatches indicated the presence of the critical region (see FIGS. 1g and 7a). As described below, the Cas13d critical region may have been masked in previous studies on RfxCas13d which used four consecutive mismatches.

As used herein, the term “match” or any other grammatical variation thereof, when referring to two nucleotide residues in one or two nucleic acid molecule(s), means a nt residue (which may be a RNA or a DNA) is complementary to the other (which may also be a RNA or a DNA). As it is known to one of skill in the art, guanine is the complementary base of cytosine, and adenine is the complementary base of thymine in DNA and of uracil in RNA. The nucleotide residues matching with each other, for example, in a secondary structure (observed or predicted) of the nucleic acid molecule(s) are a pair of nucleotide residues (nt), or paired nt. In a further embodiment, if no matching nt in the nucleic acid molecule(s), the nt is then referred to as an unpaired nt or a mismatch.

Hybridization is the process of complementary base pairs (nucleotide residues) binding to form a double helix. The term “hybridization” or any other grammatical variation hereof refers to at least two regions from one single nuclei acid molecule or of two or more nucleic acid molecules which comprises at least one nucleotide residue in one region matches a nucleotide residue in another region. In one embodiment, each of the nt in the first region matches to a nt in the second region. In a further embodiment, each of the nt in the first region matches to each of the nt in the second region. In another embodiment, one or more mismatch(es) may be found between two regions, for example one mismatch, two mismatches, two consecutive mismatches, two nonconsecutive mismatches, three or more mismatches (consecutive or nonconsecutive).

Nucleic acid secondary structure is the base pairing interactions within a single nucleic acid polymer or between two polymers. It can be represented as a list of bases which are paired in a nucleic acid molecule. Nucleic acid secondary structure can be determined from atomic coordinates (tertiary structure) obtained by X-ray crystallography, often deposited in the Protein Data Bank. Current methods include 3DNA/DSSR and MC-annotate. Methods for nucleic acid secondary structure prediction are also available, for example those relying on a nearest neighbor thermodynamic model. A common method to determine the most probable structures given a sequence of nucleotides makes use of a dynamic programming algorithm that seeks to find structures with low free energy. The lower the free energy is, the more stable the secondary structure is. Thus, minimum free energy (MFE) has been used in characterizing a secondary structure. In one embodiment, minimum free energies (MFEs) of a crRNA secondary structure was derived using RNAfold [--gquad] on the full-length crRNA sequence. See, ref 7. Also, a MFE of a secondary structure form by two regions hybridizing to each other (for example a target RNA and it corresponding guide) is referred to as a hybridization MFE. In one embodiment, in order to calculate the hybridization MFE between regions of a target RNA and is corresponding guide, Target RNA unpaired probability (accessibility) was calculated using RNAplfold [-L 4⊙ -W 8⊙ -u 5⊙ ] as described previously⁸. RNA-RNA-hybridization was calculated using RNAhybrid [-s -c] using the di-nucleotide frequency derived from the target sequence⁹. We calculated the RNA-hybridization minimum free energy for each spacer RNA nucleotide position p over the distance d to the position p+d with its cognate target sequence.

Further, G-quadruplex is a secondary structure formed in nucleic acid by sequences that are rich in guanine. They are helical structures containing guanine tetrads that can form from one, two or four strands. Four guanine bases can associate through Hoogsteen hydrogen bonding to form a square planar structure called a guanine tetrad (G-tetrad or G-quartet), and two or more guanine tetrads (from G-tracts, continuous runs of guanine) can stack on top of each other to form a G-quadruplex. G-quadruplex structures can be computationally predicted from DNA or RNA sequence motifs or other method available publicly or commercially. Generally, a simple pattern match is used for searching for possible intrastrand quadruplex forming sequences: d(G3+N1-7G3+N1-7G3+N1-7G3+), where N is any nucleotide base (including guanine). See, for example, rank-Kamenetskii M D, Mirkin S M (1995). “Triplex DNA structures”. Annual Review of Biochemistry. 64 (9): 65-95. In one embodiment, RNAfold may be used to determine a presence or absence of a G-quadruplex.

A “nucleic acid” or a “nucleotide”, as described herein, can be RNA, DNA, or a modification thereof, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. In certain embodiments, the terms “nucleotide” “nucleic acid” “nucleotide residue” and “nucleic acid residue” are used interchangeably, referring to a nucleotide in a nucleic acid polymer. In a further embodiment, consecutive nucleotide residues refer to nucleotide residues in a contiguous region of a nucleic acid polymer.

A nucleic acid molecule (RNA or DNA) or a nucleotide therein may be modified or edited. In one embodiment, such modification or edition includes 5′ capping, 3′ polyadenylation, and RNA splicing. In another embodiment, the modification or edition includes methylation (for example on a A residue resulting in a m⁶A), demethylation (for example, on a m⁶A, optionally via a RNA demethylase, including hut not limited to ALKBH5), deamination (for example, from adenosine (A) to inosine (I), optionally via a tRNA-specific adenosine deaminase (ADAT), or from C to U, optionally via a pentatricopeptide repeat (PPR) protein), or amination (for example, from U to C or from G to A).

Ribonucleic acid (RNA) is a polymeric molecule essential in various biological roles in coding, decoding, regulation and expression of genes. As used herein, RNA may refer to a CRISPR guide RNA, a messenger RNA (mRNA), a mitochondrial RNA, short hairpin RNAi (shRNAi), small interfering RNA (siRNA), a mature mRNA, a primary transcript mRNA (pre-mRNA), a ribosomal RNA (rRNA), a 5.8S rRNA, a 5S rRNA, a transfer RNA (tRNA), a transfer-messenger RNA (tmRNA), an enhancer RNA (eRNA), a small interfering RNA (siRNA), a microRNA (miRNA), a small nucleolar RNA (snoRNA), a Piwi-interacting RNA (piRNA), a tRNA-derived small RNA (tsRNA), a small rDNA-derived RNA (srRNA), a non-coding RNA (ncRNA), long (intergenic) non-coding RNA (lincRNA/lncRNA), a single-stranded RNA (ssRNA), a circular RNA (circRNA), a vault RNA (vRNA/vtRNA), a SmY RNA, a double-stranded RNA (dsRNA), a small Cajal body-specific RNA (scaRNA), an antisense RNA (aRNA/asRNA), a ribonuclease RNA (e.g. RNase P), a non-coding regulatory RNA (e.g. 7SK RNA), RNA-viruses or single stranded DNA. In one embodiment, the target RNA is an endogenous RNA. Additionally, or alternatively, the target RNA comprises/is a CDS. In another embodiment, the target RNA comprises/is a UTR (including a 5′ UTR or a 3′ UTR). In yet another embodiment, the target RNA comprises/is an intron.

As used herein, deoxyribonucleic acid (DNA) is a polymeric molecule formed by deoxyribonucleic acid, including, but not limited to, genomic DNA, double-strand DNA, single-strand DNA, DNA packaged with a histone protein, complementary DNA (cDNA which is reverse-transcribed from a RNA), mitochondrial DNA, and chromosomal DNA.

In one embodiment, the method(s) as disclosed herein is genome-wide. For example, a target RNA may be any RNA from the whole genome. In one embodiment, an off-target RNA may be any other RNA except the target RNA from the whole genome. As used herein, a genome refers to the total genetic material (e.g., DNA and RNA) of an organism.

A “vector” as used herein is a biological or chemical moiety comprising a nucleic acid sequence which can be introduced into an appropriate cell for replication or expression of said the nucleic acid sequence. Common vectors include naked DNA, phage, transposon, plasmids, viral vectors, cosmids (Phillip McClean, www.ndsu.edu/pubweb/˜mcclean/plsc731/cloning/cloning4.htm) and artificial chromosomes (Gong, Shiaoching, et al. “A gene expression atlas of the central nervous system based on bacterial artificial chromosomes.” Nature 425.6961 (2003): 917-925). One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a cell into which they are introduced (e.g., bacterial vectors having a bacterial origin of replication and episomal mammalian vectors). In certain embodiments, the vector is a lentiviral vector. Other vectors (e.g., non-episomal mammalian vectors) are integrated into the genome of a cell upon introduction into the cell, and thereby are replicated along with the cell genome.

A “viral vector” refers to a synthetic or artificial viral particle in which an expression cassette containing a nucleic acid sequence of interest is packaged in a viral capsid or envelope. Examples of viral vector include but are not limited to lentivirus, adenoviruses (Ads), retroviruses (γ-retroviruses and lentiviruses), poxviruses, adeno-associated viruses (AAV), baculoviruses, herpes simplex viruses. In one embodiment, the viral vector is replication defective. A “replication-defective virus” refers to a viral vector, wherein any viral genomic sequences also packaged within the viral capsid or envelope are replication-deficient; i.e., they cannot generate progeny virions but retain the ability to infect cells.

Pooled viral CRISPR “libraries” (in certain embodiments, lentiviral libraries) are a heterogenous population of viral transfer vectors, each containing an individual crRNA targeting a single gene in a given genome.

As used herein, the term “tag” refers to a peptide or polypeptide whose presence can be readily detected. In certain embodiments, the tag is selected from one or more of the following: a FLAG tag, a poly(His) tag, a chitin binding protein (CBP) tag, a maltose binding protein (MBP) tag, a Strep tag, a glutathione-S-transferase (GST) tag, a thioredoxin (TRX) tag, a poly(NANP) tag, a V5 tag, a HA tag, a Spot tag, a T7 tag, a NE tag, a fluorescence tag, a Green Fluorescent Protein (GFP) tag, and a MYC tag. In one embodiment, the FLAG tag has a sequence of DYKDDDK, SEQ ID NO:47. In certain embodiments, the tag is a florescent protein such as Green fluorescent protein (GFP).

A “reporter molecule”, which is used to indicate the presence of a molecule to which it is conjugated (for example, a crRNA, a nucleic acid molecule, a protein, or a Cas13d), is readily known by one of skill in the art. In one embodiment, the reporter molecule may be a tag or a nucleic acid molecule encoding a tag. In another embodiment, the reporter molecule may be an enzyme or a nucleic acid molecule expressing the enzyme, such as an E. coli lacZ enzyme, or a chloramphenicol acetyltransferase (CAT), or a luciferase.

As used herein, the term “selectable marker” refers to a molecule, a peptide or polypeptide whose presence can be readily detected in a target cell when selective pressure is applied to the cell. In certain embodiments, the selectable marker is a puromycin resistance gene, a kanamycin resistance gene, a chloramphenicol resistance gene, a blasticidin S resistance gene, a geneticin resistance gene, a hygromicin resistance gene, an ampicillin resistance gene, a tetracycline resistance gene, or a G418 resistance gene.

The term “target cell” may refer to any cell of interest. Thus, a target cell may refer to a cell having a target RNA or suspected of having a target RNA. In certain embodiments herein, the term “target cell” refers to a cell of various mammalian species. In one embodiment, the target cell is a mammalian cell. In a further embodiment, the target cell might be a eukaryotic cell, a prokaryotic cell, an embryonic stem cell, a cancer cell, a neuronal cell, an epithelial cell, an immune cell, an endocrine cell, a muscle cell, an erythrocyte, or a lymphocyte.

The term “mammal” or grammatical variations thereof, are intended to encompass a singular “mammal” and plural “mammals,” and includes, but is not limited to, humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; wild animals, such as bears, domesticated animals, livestock and laboratory animals. In some preferred embodiments, a mammal is a human.

As used herein, the term “subject” includes any mammal in need of these methods or compositions, including particularly humans. The subject may be male or female.

As used herein, the terms “therapy”, “treatment” and any grammatical variations thereof shall mean any of prevention, delay of outbreak, reducing the severity of the disease symptoms, and/or removing the disease symptoms (to cure) in a subject in need.

The Cas13d protein is a Class 2, Type VI CRISPR effector guided by a single RNA (crRNA). Two higher eukaryotes and prokaryotes nucleotide-binding (HEPN) domains have been found in the Cas13d, flanking a helical domain. See, for example, WO 2019/010384 A1, US 2019/0169595A1, Zhang C, et al. (2018). Structural Basis for the RNA-Guided Ribonuclease Activity of CRISPR-Cas13d. Cell 175, 212-223.e217, golden.com/wiki/CRISPR-Cas13d, and zlab.bio/cas13, which publication is incorporated herein by reference in its entirety. While the term Class 2, Type VI is a broader genus, of which Cas13d is exemplary, throughout the Specification, one of skill in the art would appreciate that the use of the terms “Cas13d” or “Cas13d and a variant thereof” also encompass other Class 2, Type VI proteins, and the terms can be interchangeable. Cas13d and a variant thereof includes, e.g., a wild type or naturally occurring Cas13d protein, an ortholog of a Cas13d, a functional variant thereof, or another modified variant as disclosed.

Orthologs are genes in different species that evolved from a common ancestral gene by speciation. Normally, orthologs retain the same function in the course of evolution. In some embodiments, the Cas13d is selected from a RfxCas13d from Ruminococcus flavefaciens strain XPD3002, an AdmCas13d from Anaerobic digester metagenome 15706, EsCas13d from Eubacterium siraeum DSM15702, P1E0Cas13d from Gut metagenome assembly P1E0-k21, UrCas13d from Uncultured Ruminoccocus sp., RffCas13d from Ruminoccocus flavefaciens FD1, and RaCas13d from Ruminoccocus albus. In one embodiment, the Cas13d protein is a RfxCas13d or a variant thereof. The amino acid sequences of the Cas13d orthologs are publically available. In one embodiment, the Cas13d has an amino acid sequence as provided by a Protein Data Bank (PDB) accession number 6OAW_B or 6OAW_A or 6E9F_A or 6E9E_A or 6IV9_A, or an amino acid sequence as provided by the UniProtKB identifier B0MS50 (B0MS50_9FIRM) or A0A1C5SD84 (A0A1C5SD84_9FIRM). Each of the sequences of these references is incorporated by reference herein in its entirety.

In one embodiment, a variant of Cas13d may be a functional variant of the Cas13d protein which is a protein or a polypeptide which shares the same biological function with Cas13d. A functional variant of the Cas13d protein might be a Cas13d protein with 1, 2, 3, 4, 5, 6, 7, 8, 9, 10, 11, 12, 13, 14, 15, 16, 17, 18, 19, about 20, about 25, about 30, about 35, about 40, about 45, about 50, about 60, about 70, about 80, about 90, about 100, about 110, about 120, about 130, about 140, about 150, about 160, about 170, about 180, about 200, about 220, about 240, about 260, about 280, about 300, about 330, about 360, about 390 or more conserved amino acid substitution(s). Identifying an amino acid for a possible conserved substitution, determining a substituted amino acid, as well as the methods and techniques involved in incorporating the amino acid substitution into a protein are well-known to one of skill in the art. See, sift.jcvi.org/and (Ng & Henikoff, Predicting the Effects of Amino Acid Substitutions on Protein Function, 2006; Ng & Henikoff, Predicting the effects of coding non-synonymous variants on protein function using the SIFT algorithm, 2009; Ng P C, 2003; Ng & Henikoff, Accounting for Human Polymorphisms Predicted to Affect Protein Function, 2002; Sim, et al., 2012; Sim, et al., 2012), each of which is incorporated herein by reference in its entirety.

In another embodiment, a Cas13d variant is a Cas13d protein mutated to increase or decrease or abolish its nuclease activity. Without wishing to be bound by the theory, although we specifically sought to define rules for active Cas13d, our model is transferable to inactive (nuclease-null or dead) Cas13d effector proteins, as the main feature is defined by crRNA folding/accessibility. In yet another embodiment, a Cas13d variant is a Cas13d protein conjugated to another molecule, for example, a reporter molecule, a splicing factor, an enzyme editing or modifying an RNA, a polyA factor, a nuclear localization signal (NLS), organelle specific signal, or a cytosolic signal or a nuclear-export signal (NES).

As used herein, a nuclear localization signal or sequence (NLS) is an amino acid sequence that ‘tags’ a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Further, a cytosolic signal directs a protein into cytosol of the target cell while an organelle specific signal guides a protein into a specific organelle (for example, cytoplasm, ribosome, or mitochondria).

In one embodiment, used herein is a Cas13d variant having a nuclear localization signal (NLS). In a further embodiment, one amino acid sequence of the Cas13d variant is listed below,

(SEQ ID NO: 49)
MSPKKKRKVEASIEKKKSFAKGMGVKSTLVSGSKVYMTTFAEGSDARLEK
IVEGDSIRSVNEGEAFSAEMADKNAGYKIGNAKFSHPKGYAVVANNPLYT
GPVQQDMLGLKETLEKRYFGESADGNDNICIQVIHNILDIEKILAEYITN
AAYAVNNISGLDKDIIGFGKFSTVYTYDEFKDPEHHRAAFNNNDKLINAI
KAQYDEFDNFLDNPRLGYFGQAFFSKEGRNYIINYGNECYDILALLSGLR
HWVVHNNEEESRISRTWLYNLDKNLDNEYISTLNYLYDRITNELTNSFSK
NSAANVNYIAETLGINPAEFAEQYFRFSIMKEQKNLGFNITKLREVMLDR
KDMSEIRKNHKVFDSIRTKVYTMMDFVIYRYYIEEDAKVAAANKSLPDNE
KSLSEKDIFVINLRGSFNDDQKDALYYDEANRIWRKLENIMHNIKEFRGN
KTREYKKKDAPRLPRILPAGRDVSAFSKLMYALTMFLDGKEINDLLTTLI
NKFDNIQSFLKVMPLIGVNAKFVEEYAFFKDSAKIADELRLIKSFARMGE
PIADARRAMYIDAIRILGTNLSYDELKALADTFSLDENGNKLKKGKHGMR
NFIINNVISNKRFHYLIRYGDPAHLHEIAKNEAVVKFVLGRIADIQKKQG
QNGKNQIDRYYETCIGKDKGKSVSEKVDALTKIITGMNYDQFDKKRSVIE
DTGRENAEREKFKKIISLYLTVIYHILKNIVNINARYVIGFHCVERDAQL
YKEKGYDINLKKLEEKGFSSVTKLCAGIDETAPDKRKDVEKEMAERAKES
IDSLESANPKLYANYIKYSDEKKAEEFTRQINREKAKTALNAYLRNTKWN
VIIREDLLRIDNKTCTLFRNKAVHLEVARYVHAYINDIAEVNSYFQLYHY
IMQRIIMNERYEKSSGKVSEYFDAVNDEKKYNDRLLKLLCVPFGYCIPRF
KNLSIEALFDRNEAAKFDKEKKKVSGNSGSGPKKKRKVAAAYPYDVPDYA

In one embodiment, a Cas13d or a variant thereof can further comprise a nuclear localization signal (NLS). In another embodiment, the Class 2, Type VI protein, e.g., Cas13d, can further encompass or be fused to a cytosolic signal or a nuclear-export signal (NES). In still another embodiment, the Cas13d or a variant thereof is fused to an endoplasmic reticulum localization element (see plasmid 79055, labeled ERM-APEX2 by Addgene at www.addgene.org/79055/). In yet a further embodiment, the Cas13d or a variant thereof is fused to an Outer Mitochondrial membrane localization element (See, the APEX2-OMM plasmid #79056 described by Addgene at www.addgene.org/79056/). In another embodiment, the Cas13d or a variant thereof is fused to a Mitochondria localizing element (such as plasmid 72480 Mito-V5-APEX2 described by Addgene atwww.addgene.org/72480/). In still other embodiments, the Cas13d or a variant thereof is fused to a Nucleolus localizing element (NIK3x), a Nuclear lamina localizing element (LMNA) or a Nuclear pore complex localizing element (SENP2). See, e.g., Fazal, F M et al, 2019 Atlas of Subcellular RNA Localization Revealed by APEX-Seq, Cell, 178:473-490, incorporated by reference herein.

A variety of algorithms and/or computer programs are well known in the art or commercially available for alignment of multiple amino acid sequences (e.g., BLAST, ExPASy; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

In some embodiments, the nucleic acid sequence encoding Cas13d or a variant thereof may be codon-optimized for expression in eukaryotic cell, such as mammalian cells. Methods of codon-optimization are known and have been described previously (e.g. International patent publication No. WO 96/09378). A sequence is considered codon-optimized if at least one non-preferred codon as compared to a wild type sequence is replaced by a codon that is more preferred. Herein, a non-preferred codon is a codon that is used less frequently in an organism than another codon coding for the same amino acid. A codon that is more preferred is a codon that is used more frequently in a target cell than a non-preferred codon. The frequency of codon usage for a specific organism can be found in codon frequency tables, such as in www.kazusa.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression. Numerous different nucleic acid molecules can encode the same polypeptide as a result of the degeneracy of the genetic code.

Skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g. GeneArt™, GenScript®, Life Technologies™, Eurofins).

In one embodiment, the Cas13d coding sequence is operably linked to a regulatory element to ensure expression in a target cell. In a further embodiment, the promoter is an inducible promoter, such as a doxycycline inducible promoter. In a preferred embodiment, the regulatory element(s) comprises an RNA pol II promoter. A RNA pol II promoter is a promoter that is sufficient to direct accurate initiation of transcription by the RNA polymerase II machinery, wherein the RNA polymerase II (RNAP II and Pol II) is a RNA polymerase found in the nucleus of eukaryotic cells, catalyzing the transcription of DNA to synthesize precursors of messenger RNA (mRNA) and most small nuclear RNA (snRNA) and microRNA.

A variety of Polymerase II promoters that can be used within the compositions and methods described herein are publicly or commercially available to a skilled artisan, for example, viral promoters obtained from the genomes of viruses including promoters from polyoma virus, fowlpox virus (UK 2,211,504), adenovirus (such as Adenovirus 2 or 5), herpes simplex virus (thymidine kinase promoter), bovine papilloma virus, avian sarcoma virus, cytomegalovirus (CMV), a retrovirus (e.g., MoMLV, or RSV LTR), Hepatitis-B virus, Myeloproliferative sarcoma virus promoter (MPSV), VISNA, and Simian Virus 40 (SV40); other heterologous mammalian promoters including the actin promoter, β-actin promoter, immunoglobulin promoter, heat-shock protein promoters, human Ubiquitin-C promoter, PGK promoter. Additional promoters are readily known and available. See, e.g., (Kadonaga, 2012), WO 2014/15134, and WO 2016/054153. In one particular embodiment, the promoter is a CMV promoter. In a further embodiment, the promoter is an EF-1 Alpha Short (EFS) promoter, or a Tet operator (tetO) promoter

The term “regulatory element” or “regulatory sequence” refers to expression control sequences which are contiguous with the nucleic acid sequence of interest (for example, a Cas13d coding sequence or a sequence for expressing a crRNA) and expression control sequences that act in trans or at a distance to control the nucleic acid sequence of interest. As described herein, regulatory elements comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Also, see Goeddel; Gene Expression Technology: Methods in Enzymology 185, Academic Press, San Diego, Calif. (1990). Regulatory sequences include those which direct constitutive expression of a nucleic acid sequence in many types of target cell and those which direct expression of the nucleic acid sequence only in certain target cells (e.g., tissue-specific regulatory sequences). Furthermore, the Cas13d can be delivered by way of a vector comprising a regulatory sequence to direct synthesis of the Cas13d at specific intervals, or over a specific time period. It will be appreciated by those skilled in the art that the design of the vector can depend on such factors as the choice of the target cell, the level of expression desired, and the like.

As used herein, “operably linked” sequences or sequences “in operative association” include both expression control sequences that are contiguous with the nucleic acid sequence of interest (for example, a Cas13d coding sequence or a sequence for expressing a crRNA) and expression control sequences that act in trans or at a distance to control the nucleic acid sequence of interest.

As used herein, “polyadenylation” is the addition of a poly(A) tail to a messenger RNA, which is important for the nuclear export, translation, and stability of mRNA. Examples of suitable polyA sequences include, e.g., Rabbit globin poly A, SV40, SV50, bovine growth hormone (bGH), human growth hormone, and synthetic polyAs.

Optionally, the nucleic acid sequence encoding a Cas13d protein further comprises a reporter gene or a nucleic acid encoding a selectable marker, which may include sequences encoding geneticin, hygromicin, ampicillin or purimycin resistance, among others. A reporter gene, which is used as an indication of whether the Cas13d coding sequence has been incorporated into and/or expressed as a functional protein in the target cell or not, is readily selected by one of skill in the art, including without limitation, the E. coli lacZ gene, the chloramphenicol acetyltransferase (CAT) gene, or a gene encoding a fluorescent protein such as Green fluorescent protein (GFP).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically acceptable” refers to molecular entities and compositions that do not produce an allergic or similar untoward reaction when administered to a subject. Delivery vehicles such as lipid particle, liposomes, nanocapsules, nanosphere, nanoparticle, microparticles, microspheres, lipid particles, vesicles, and the like, may be used for the introduction of the compositions of the present invention into suitable target cells.

By “biological sample” is meant any biological fluids, cells or tissues of a subject that is suitable for use, such as, for example, cell-containing body fluids such as blood, sperm, cerebral spinal fluid, saliva, sputum or urine, leukocyte fractions, buffy coat, feces, swabs, puncture fluids, skin fragments, whole organisms or parts thereof, organs, organ fragments, tissues and tissue parts of a subject. Still other suitable samples are in the form of sections, biopsies, fine needle aspirates or tissue sections, isolated cells, for example in the form of adherent or suspended cell cultures, plants, plant parts, plant tissues from the fractions may be carried out at the same time or one or plant cells, bacteria, viruses, yeasts and fungi, without being limited thereto. In one embodiment, the biological sample contains a target RNA. In one embodiment, a suitable biological sample is a tissue section from human tissue, such as a tumor.

The terms “a” or “an” refers to one or more. For example, “an expression cassette” is understood to represent one or more such cassettes. As such, the terms “a” (or “an”), “one or more,” and “at least one” are used interchangeably herein. In certain embodiment, the term “one or more” refers to any integer from one to the maximum including any integer therebetween.

The terms “another”, “first”, “second”, “third”, “fourth”, “fifth” and “sixth” are used throughout this specification as reference terms to distinguish between various forms and components of the compositions and methods, for example, first or second promoter.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

As described herein, the terms “reduce” “decrease” “alleviate” “ameliorate” “improve” “delay” “earlier” “low” “high” “mitigate”, any grammatical variation thereof, or any similar terms indication a change, means a variation of about 5 fold, about 2 fold, about 1 fold, about 90%, about 80%, about 70%, about 60%, about 50%, about 40%, about 30%, about 20%, about 10%, about 5% compared to a reference (e.g., a guide generated without using the disclosed methods, or a non-targeting control), unless otherwise specified.

Also, it is noted that any range as disclosed herein, (for example, an MFE range, a hybridization MFE range, a nucleotide (nt) range, a percentage range, or a log₁₀ value range) includes the endpoint and every number/nt/percentage/value therebetween, unless specified.

It is also noted that any embodiment listed with respect to a crRNA, a nucleic acid molecule, a vector, a library, a composition, any other component, a method, or a use, may be combined with any other embodiments with respect to a crRNA, a nucleic acid molecule, a vector, a library, a composition, any other component, a method, or a use.

B. Method of Designing, Generating and Ranking crRNA(s)

In one aspect, provided is a method for generating and selecting a crRNA which is capable of forming a complex with a CRISPR-associated protein 13d (Cas13d) or a variant thereof, and directing the complex to a target RNA. The method comprises the following steps:

(1) randomly designating a potential hybridization region in the target RNA;

(2) designing a guide which is capable of hybridizing to the hybridization region, and designing a crRNA sequence comprising the guide and a DR stem loop accordingly;

(3) ranking each crRNA based on features of the crRNA and its corresponding target RNA.

In one embodiment, the designated target RNA is longer than 87 nucleotides (nt). In a further embodiment, the designated target RNA is longer than 100 nt or 200 nt or 300 nt or 400 nt or 500 nt. In certain embodiments, the ranking does not consider a protospacer in the target RNA for directing the complex. In a further embodiment, nt 15 to nt 21 (or nt 17 to 18 or nt 18) of the crRNA matching with its corresponding hybridization region of the target RNA without mismatches ranks higher than those with mismatches. In yet a further embodiment, crRNA having three or more mismatches to its corresponding target RNA ranks lower comparing to those having 0, 1 or 2 mismatches.

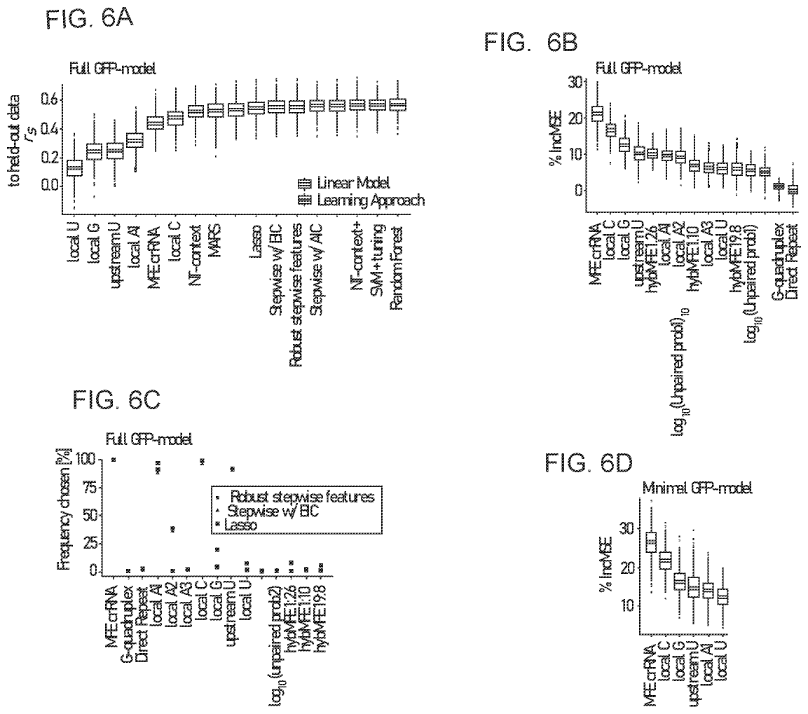

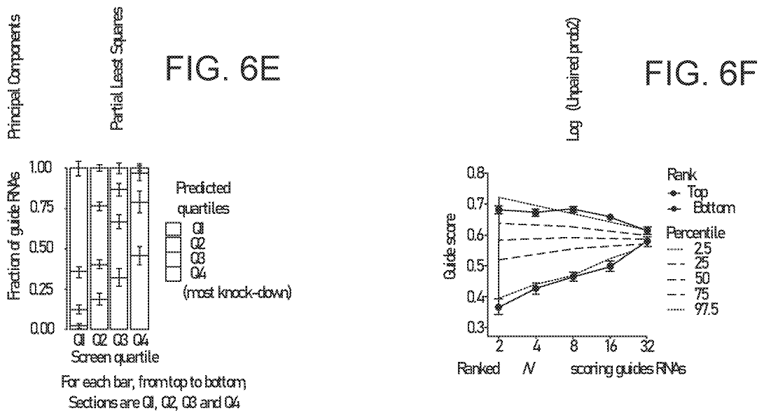

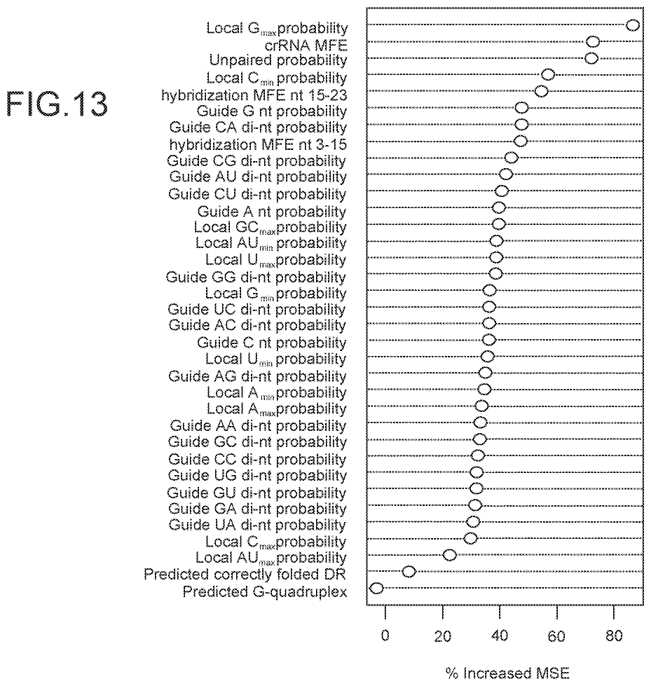

In one embodiment, crRNA with a feature falling in the range of a positively-correlated feature and out of the range of a negatively-correlated feature. In one embodiment, the features are listed in Tables 2 and 4-7 and FIGS. 6 and 13. In a further embodiment, ranges are provided in Table 2. ranks higher. Without wishing to be bound by the theory, a G dependent stable structure (for example a G-quadruplex) within the crRNA renders the crRNA inaccessible for Cas13d. Additionally or alternatively, of a perfect matching crRNA having a higher minimum free energy (MFE) ranks higher.

In certain embodiments, (a) minimum free energy (MFE) value of the crRNA is considered in the ranking step. In one embodiment, a crRNA having an MFE value of (a) within the following range ranks higher than those falling out of the following range: from −22.8 to −12.8, or from −20.9 to −14.3, or from −23.4 to −14.5, or from −18.7 to −15.9, or about −17.1, or about −17.3, each of the value ranges including the endpoints and all numbers therebetween. In one embodiment, the MFE is calculated via a publicly available software of predicting RNA secondary structure for single stranded RNAs (such as crRNAs), for example, RNAfold. See, Lorenz, R. et al. ViennaRNA Package 2.0. Algorithms Mol. Biol. 6, 26 (2011).

In certain embodiments, a crRNA having a DR stem loop which is about 30 nt long ranks higher. In certain embodiments, a crRNA having a 30 nt or 3 1 nt long DR stem loop ranks higher compared to those having a DR stem loop of other lengths.

Based on its secondary structure, the DR stem loop is composed of, from the 5′ end to the 3′ end, a 5′ end, a stem loop which is capable of forming a self-hybridizing structure via paired nucleotides matching with each other, and a 3′ end. The 5′ and 3′ ends of the DR stem loop do not match to the target RNA or any nucleotide of the DR stem loop. In one embodiment, a crRNA having a stem loop comprising 4 unpaired nucleotides in the middle of the sequence forming a loop ranks higher. In yet a further embodiment, a crRNA having a stem loop with an additional two unpaired nucleotide residues in the stem loop forming a bulge, ranks higher. In one embodiment, a crRNA ranks higher if the 5′ end of its DR stem loop is one unpaired nucleotide.

In certain embodiments, the ranking is further determined based on the feature of (g): presence of a DR stem loop having a motif selected from the following:
(I) 5′-(₁ (₂ (₃ (₄ (₅ (₆. (₇ (₈ (₉ . . . )₉ )₈ )₇. )₆ )₅ )₄ )₃ )₂ )₁-3′,
(II) 5′-. (₁ (₂ (₃ (₄ (₅. (₆ (₇ (₈ . . . )₈ )₇ )₆. )₅ )₄ )₃ )₂ )₁.-3′,
(III) 5′- . . . (₁ (₂ (₃ (₄. (₅ (₆ (₇ . . . )₇ )₆ )₅. )₄ )₃ )₂ )₁ . . . -3′,
(IV) 5′- . . . (₁ (₂ (₃. (₄ (₅ (₆ . . . )₆ )₅ )₄. )₃ )₂ )₁ . . . -3′, and
(V) 5′-(₁ (₂ (₃ (₄ (₅ (₆ (₇ (₈ (₉ (₁₀ . . . )₁₀ )₉ )₈ )₇ )₆ )₅ )₄ )₃ )₂ )₁- 3′,

As used herein, “(ₙ” and “)ₙ” represent a pair of nucleotides matching with each other, and “.” represents an unpaired nucleotide in bulge or loop. As defined above, the part from (₁ to )₁ is the self-hybridizing stem loop of the DR stem loop while the nt at the 5′ or 3′ of the self-hybridizing stem loop are the 5′ end or 3′ end, respectively.

In a further embodiment, a crRNA forming an effective guide RNA and having a higher ranking is provided with a DR stem loop sequence as recited in TABLE 9 below. With sequence (I) being the sequence found in Ruminococcus flavefaciens (Rfx), we found that the DR sequence modifications II and III showed improvement relative to sequence I. While we introduced specific sequence changes (e.g., replaced nucleotide 1 from A 4 U for sequence I to sequence II), we anticipate that any nucleotide replacement with a similar consequential effect likely yields similar benefits. For example, replacing the A nucleotide in position 1 with either of U or C and to some degree G will similarly disrupt base pair capabilities between nucleotide 1 and the U at position 24. Therefore, we indicate nucleotide changes according to IUPAC nomenclature in addition to the conventional abbreviations A for Adenine, C for Cytosine, G for Guanine and T (or U) for Thymine (or Uracil) by use of the abbreviations: R for A or G; Y for C or T(or U); S for G or C; W for A or T(or U); K for G or T(or U); M for A or C; B for C or G or T(or U); D for A or G or T(or U); H for A or C or T(or U); V for A or C or G; N for any base; . or - to represent a nucleotide gap.

In one embodiment, changes in the nucleotide at position 1 or 24 can have the same consequence of base pair disruption. Thus, any change introduced for the five-prime base pair mate can be mirrored for the three-prime mate. For example:

### UACCCCUACCAACUGGUCGGGGUUUGAAAC SEQ ID NO:2 and

AACCCCUACCAACUGGUCGGGGUAUGAAAC SEQ ID NO: 46 are anticipated to yield the same effect.

In another embodiment, removing nucleotides from the DR 5′ end or the addition of hindering nucleotides 5′ to the sequence is predicted to alter the DR function in the same way. For example,

### UACCCCUACCAACUGGUCGGGGUUUGAAAC SEQ ID NO: 2 and

ACCCCUACCAACUGGUCGGGGUUUGAAAC SEQ ID NO: 20 likely yield the same effect. In still another aspect, nucleotide removal or addition, alone and in conjunction, in sequences I-VII are anticipated to produce effective DR stem loops for effective guides. The use of such DR stem loops are also anticipated to increase the efficacy of binding of even mismatched crRNA.

Thus Table 9 provides exemplary DR stem loops comprising one of the following sequences or a modification thereof.

TABLE 9

| | | SEQ |
|---|---|---|
| DR Ref | | ID |
| No. | DR Sequence | NO: |
| (I) | AACCCCUACCAACUGGUCGGGGUUUGAAAC | 1 |
| (II) | UACCCCUACCAACUGGUCGGGGUUUGAAAC | 2 |
| (III) | UUCCCCUACCAACUGGUCGGGGUUUGAAAC | 3 |
| (IV) | UUUCCCUACCAACUGGUCGGGGUUUGAAAC | 4 |
| (V) | UUACCCUACCAACUGGUCGGGGUUUGAAAC | 5 |
| (VI) | AACCCCGACCAACUGGUCGGGGUUUGAAAC | 6 |
| (VII) | UACCCCGACCAACUGGUCGGGGUUUGAAAC | 7 |
| (VIII) | UUCCCCGACCAACUGGUCGGGGUUUGAAAC | 8 |
| (IX) | UUUCCCGACCAACUGGUCGGGGUUUGAAAC | 9 |
| (X) | AACCCCUACCAACUGGUAGGGGUUUGAAAC | 10 |
| (XI) | UACCCCUACCAACUGGUAGGGGUUUGAAAC | 11 |
| (XII) | UUCCCCUACCAACUGGUAGGGGUUUGAAAC | 12 |
| (XIII) | UUUCCCUACCAACUGGUAGGGGUUUGAAAC | 13 |
| (IIₐₗₜ₁) | BACCCCUACCAACUGGUCGGGGUUUGAAAC | 14 |
| (IIIₐₗₜ₁) | NBCCCCUACCAACUGGUCGGGGUUUGAAAC | 15 |
| (IVₐₗₜ₁) | NNDCCCUACCAACUGGUCGGGGUUUGAAAC | 16 |
| (Vₐₗₜ₁) | AACCCCUACCAACUGGUCGGGGUVUGAAAC | 17 |
| (VIₐₗₜ₁) | UUACCCUACCAACUGGUCGGGGVNUGAAAC | 18 |
| (VIIₐₗₜ₁) | AACCCCUACCAACUGGUCGGGHNNUGAAAC | 19 |
| (VIIIₐₗₜ₁) | -ACCCCUACCAACUGGUCGGGGUUUGAAAC | 20 |
| (IXₐₗₜ₁) | --CCCCUACCAACUGGUCGGGGUUUGAAAC | 21 |
| (Xₐₗₜ₁) | ---CCCUACCAACUGGUCGGGGUUUGAAAC | 22 |
| (XIIₐₗₜ₁) | (N)ₙAACCCCUACCAACUGGUCGGGGUUUGAAAC | 23 |
| (Iₐₗₜ₂) | AACCCCVACCAACUGGUCGGGGUUUGAAAC | 24 |
| (IIₐₗₜ₂) | BACCCCVACCAACUGGUCGGGGUUUGAAAC | 25 |
| (IIIₐₗₜ₂) | NBCCCCVACCAACUGGUCGGGGUUUGAAAC | 26 |
| (IVₐₗₜ₂) | NNDCCCVACCAACUGGUCGGGGUUUGAAAC | 27 |
| (Vₐₗₜ₂) | AACCCCVACCAACUGGUCGGGGUVUGAAAC | 28 |
| (VIₐₗₜ₂) | UUACCCVACCAACUGGUCGGGGVNUGAAAC | 29 |
| (VIIₐₗₜ₂) | AACCCCVACCAACUGGUCGGGHNNUGAAAC | 30 |
| (VIIIₐₗₜ₂) | -ACCCCVACCAACUGGUCGGGGUUUGAAAC | 31 |
| (IXₐₗₜ₂) | --CCCCVACCAACUGGUCGGGGUUUGAAAC | 32 |
| (Xₐₗₜ₂) | ---CCCVACCAACUGGUCGGGGUUUGAAAC | 33 |
| (XIIₐₗₜ₂) | (N)ₙAACCCCVACCAACUGGUCGGGGUUUGAAAC | 34 |
| (Iₐₗₜ₃) | AACCCCUACCAACUGGUDGGGGUUUGAAAC | 35 |
| (IIₐₗₜ₃) | BACCCCUACCAACUGGUDGGGGUUUGAAAC | 36 |
| (IIIₐₗₜ₃) | NBCCCCUACCAACUGGUDGGGGUUUGAAAC | 37 |
| (IVₐₗₜ₃) | NNDCCCUACCAACUGGUDGGGGUUUGAAAC | 38 |
| (Vₐₗₜ₃) | AACCCCUACCAACUGGUDGGGGUVUGAAAC | 39 |
| (VIₐₗₜ₃) | UUACCCUACCAACUGGUDGGGGVNUGAAAC | 40 |
| (VIIₐₗₜ₃) | AACCCCUACCAACUGGUDGGGHNNUGAAAC | 41 |
| (VIIIₐₗₜ₃) | -ACCCCUACCAACUGGUDGGGGUUUGAAAC | 42 |
| (IXₐₗₜ₃) | --CCCCUACCAACUGGUDGGGGUUUGAAAC | 43 |
| (Xₐₗₜ₃) | ---CCCUACCAACUGGUDGGGGUUUGAAAC | 44 |
| (XIIₐₗₜ₃) | (N)ₙAACCCCUACCAACUGGUDGGGGUUUGAAAC | 45 |

In yet a further embodiment, a crRNA having a DR stem loop composed of a G-residue at the 5′ end followed by one of sequences (I) to (XIII) ranks higher. Additionally or alternatively, the ranking is further determined based on a feature of (h) which is absence of a G-quadruplex in the crRNA. In certain embodiments, the presence of a G-quadruplex is determined by RNAfold. In certain embodiments, a crRNA without a G-quadruplex ranks higher.

In certain embodiments, a crRNA with a more stable hybridization between guide and its target sequence ranks lower. Such hybridization may be assessed via hybridization MFE between a target RNA and its corresponding regions of the crRNA, wherein a lower hybridization MFE indicates a more stable hybridization. Without wishing to be bound by theory, it is believed that the most stable guide-target interactions render the crRNA-Cas13d complex inactive. In one embodiment, a crRNA with a more stable hybridization between regions of the guide (which is not the full length guide) and its target sequence ranks lower.

In one embodiment, the crRNA(s) with the highest ranking is selected for directing a Cas13d-crRNA complex to a target RNA. In certain embodiments, a crRNA having a positively correlated feature as disclosed ranks higher than those without the positively correlated feature(s). In yet another embodiment, a crRNA or its corresponding target RNA having more positively correlated features within the identified ranges ranks higher. In certain embodiments, a crRNA having a negatively correlated feature as disclosed ranks lower than those without the negatively correlated feature(s). In another embodiment, a crRNA or its corresponding target RNA having more negatively-correlated features within the identified ranges ranks lower.

In certain embodiments, a crRNA ranks lower if it has an off-target activity or has a higher off-target activity. In one embodiment, an off-target activity is determined if an RNA other than the target RNA comprises the hybridization region of the target RNA, or if an RNA other than the target RNA comprises the hybridization region of the target RNA with one nucleotide residue difference outside of nt −14 to nt −20 of the target RNA; or if an RNA other than the target RNA comprises the hybridization region of the target RNA with two nonconsecutive nucleotide residue differences outside of nt −14 to nt −20 of the target RNA. In certain embodiments, the RNA other than the target RNA is termed as “off-target RNA”. In certain embodiments, the crRNA and/or the crRNA-Cas13d complex is designed to apply to a target cell. In a further embodiment, the off-target RNA also exists in the target cell. In yet a further embodiment, the off-target RNA is at least 87 nt long, or at least 100 nt long, or at least 200 nt long, or at least 300 nt long, or at least 500 nt long.

In one aspect, provided is a method for predicting on-target activity of a crRNA. The crRNA composed of a DR stem loop and a guide is capable of forming a complex with a Cas13d or a variant thereof and directing the complex to the target RNA. The method comprises characterizing one or more of the features (any one or combination of the features as disclosed herein) of a plurality of crRNAs and their corresponding target RNAs; assessing on-target activity of each of the crRNAs; constructing a model using the characterization data and the on-target activity data by a modeling method. In one embodiment, the modeling method comprises Random Forest modeling. Additionally, or alternatively, the modeling method comprises one or more of methods listed in Table 3. In a further embodiment, input of the model comprises characterization(s) of one or more of features of a crRNA and its corresponding target RNA. In yet a further embodiment, output of the model is an on-target score of the crRNA. As used herein an on-target score is an assigned number (for example, an integer, rational number or irrational number) which positively correlates to on-target activity of a crRNA.

In one embodiment, the predicting method further comprises applying the constructed model to a crRNA and generating an on-target score of the crRNA. In a further embodiment, the predicting method comprises applying the constructed model to two or more crRNAs (such as a first crRNA and a second crRNA), and generating on-target scores of the crRNAs. In yet a further embodiment, the crRNAs share the same target RNA. In one embodiment, the crRNA is capable of hybridizing to a different (overlapping or non-overlapping) hybridization region of the same target RNA. In certain embodiments, the predicting method further comprises comparing the generated on-target scores and selecting the crRNA having the higher/highest score for directing the crRNA-Cas13d complex to the target RNA.

In one embodiment, the features of a crRNA and its corresponding target RNA are one or more of the following or the ones listed in one or more of those listed in Tables 2 and 4-7 and FIGS. 6 and 13: minimum free energy (MFE) value of the crRNA; proportion of adenine (A) residues in the corresponding target RNA ranging from nucleotide (nt) −19 to nt −25; proportion of cytosine (C) residues in the corresponding target RNA ranging from nt 0 to nt −21; proportion of guanine (G) residues in the corresponding target RNA ranging from nt 0 to nt −20; proportion of uracil (U) residues in the corresponding target RNA ranging from nt 11 to nt −17; proportion of uracil (U) residues in the corresponding target RNA ranging from nt −69 to nt −86; presence or absence of a DR stem loop having a motif selected from the following: (I) 5′-(₁ (₂ (₃ (₄ (₅ (₆. (₇ (₈ (₉ . . . )₉ )₈ )₇. )₆ )₅ )₄ )₃ )₂ )₁-3′, (II) 5′-. (₁ (₂ (₃ (₄ (₅. (₆ (₇ (₈ . . . )₈ )₇ )₆. )₅ )₄ )₃ )₂ )₁. -3′, (III) 5′- . . . (₁ (₂ (₃ (₄. (₅ (₆ (₇ . . . )₇ )₆ )₅. )₄ )₃ )₂ )₁ . . . -3′, (IV) 5′- . . . (₁ (₂ (₃. (₄ (₅ (₆ . . . )₆ )₅ )₄. )₃ )₂ )₁ . . . -3′, and (V) 5′-(₁ (₂ (₃ (₄ (₅ (₆ (₇ (₈ (₉ (₁₀ . . . )₁₀ )₉ )₈ )₇ )₆ )₅ )₄ )₃ )₂ )₁ -3′, wherein “(ₙ” and “)ₙ” represent a pair of nucleotides which are capable of hybridizing with each other, and “.” represents an unpaired nucleotide in bulge or loop; absence or presence of a G-quadruplex in the crRNA; hybridization MFE value between (I) nt 0 to nt −25 of the target RNA and (II) its corresponding region of the crRNA; hybridization MFE value between (I) nt 0 to nt −9 of the target RNA and (II) its corresponding region of the crRNA; hybridization MFE value between (I) nt −18 to nt −25 of the target RNA and (II) its corresponding region of the crRNA; log₁₀ (probability of a nucleotide being unpaired from nt −13 to nt −33 of the target RNA and the corresponding nt of the crRNA); log₁₀ (probability of a nucleotide being unpaired from nt −27 to nt −18 of the target RNA and the corresponding nt of the crRNA); proportion of A residues in the target RNA ranging from nt −6 to nt −48; and proportion of A residues in the target RNA ranging from nt −25 to nt −6; whether each nt from nt −14 to nt −20 of the target RNA matches its corresponding region of the crRNA; whether the guide is about 23 nt long to about 33 nt long, or about 27 nt to about 30 nt long, or about 27 nt long; proportion of A residue(s) of the guide; proportion of C residue(s) of the guide; proportion of G residue(s) of the guide; proportion of U residue(s) of the guide; proportion of A or U residue(s) of the guide; proportion of G or C residue(s) of the guide; proportion of AA residues of the guide; proportion of AC residues of the guide; proportion of AG residues of the guide; proportion of AU residues of the guide; proportion of CA residues of the guide; proportion of CC residues of the guide; proportion of CG residues of the guide; proportion of CU residues of the guide; proportion of GA residues of the guide; proportion of GC residues of the guide; proportion of GG residues of the guide; proportion of GU residues of the guide; proportion of UA residues of the guide; proportion of UC residues of the guide; proportion of UG residues of the guide; and proportion of UU residues of the guide. In one embodiment, the nt numbering is based on a numbering from 5′ end of the target RNA to 3′ end recognizing the nt which is capable of matching to the guide match start as nt 0, and wherein each of the nt ranges includes endpoints.

As used herein, an on-target activity of a crRNA (i.e., efficacy of a crRNA) may refer to one or more of the following: efficacy of the crRNA in forming a complex with a Cas13d protein or a variant thereof; efficacy of the crRNA in hybridizing to the corresponding target RNA; efficacy of the crRNA in directing a Cas13d-crRNA complex to the target RNA; efficacy of the crRNA in reducing the corresponding target RNA; and enrichment or abundance or depletion of the crRNA (or the guide of the crRNA or the target RNA) after applying the crRNA and a Cas13d or a variant thereof to a cell or cell culture. As an illustrative embodiment shown in the Example, the crRNA efficacy was determined by quantifying crRNA abundances in sorted and unsorted cell populations. The value represents the log₂ fold change of sorted divided by input (for example, unsorted) counts. Higher values depict higher efficacies/efficiencies for target knockdown owed to the screen design. As used herein, an on-target score may be used to quantify the on-target activity. In one embodiment, an on-target score is an efficiency quartile as used here in (Q1 to Q4 also shown as bin1 to bin4 ). In another embodiment, an on-target score is a measured or calculated efficacy, for example, a fold change of crRNA/guide/target RNA abundance before v.s. after applying the crRNA.

In one aspect, provided is a method for predicting off-target activity of a crRNA. As disclosed herein, the crRNA is composed of a DR stem loop and a guide, and is capable of forming a complex with a Cas13d or a variant thereof and directing the complex to the target RNA. The predicting method comprises characterizing one or more of the features of a plurality of crRNAs and their corresponding target RNAs; assessing off-target activity of each of the crRNAs; and constructing a model using the characterization and the off-target activity acquired by a modeling method. In one embodiment, the modeling method comprises Random Forest modeling. In another embodiment, the modeling method comprises a deep learning model. Additionally, or alternatively, the model-constructing method comprises one or more of methods listed in Table 3. In one embodiment, input of the model comprises characterization(s) of one or more of features of a crRNA and its corresponding target RNA. Additionally or alternatively, output of the model is an off-target score of the crRNA positively correlating to off-target activity of the crRNA.

In one embodiment, the predicting method further comprises applying the constructed model to a crRNA and generating an off-target score of the crRNA. In a further embodiment, the predicting method further comprises applying the constructed model to two or more crRNA (for example, a first crRNA and a second crRNA) and generating off-target scores of the crRNAs. In yet a further embodiment, the crRNAs share the same target RNA. In one embodiment, the crRNA is capable of hybridizing to a different (overlapping or non-overlapping) hybridization region of the same target RNA. In certain embodiments, the predicting method further comprises comparing the generated off-target scores and selecting the crRNA having the lower/lowest score for directing the crRNA-Cas13d complex to the target RNA and avoiding off-target effect(s).

In certain embodiments, the features discussed with respect to the method for predicting off-target activity is any one or any combination of the features disclosed herein. In one embodiment, the features are one or more of the following: presence and absence of an off-target RNA comprises the hybridization region of the target RNA, or presence and absence of an off-target RNA comprises the hybridization region of the target RNA with one nucleotide residue difference outside of nt −14 to nt −20 of the target RNA; presence and absence of an off-target RNA comprises the hybridization region of the target RNA with two nonconsecutive nucleotide residue differences outside of nt −14 to nt −20 of the target RNA. In one embodiment, the nt numbering is based on a numbering from 5′ end of the target RNA to 3′ end recognizing the nt which is capable of matching to the guide match start as nt 0.

As used herein, an off-target activity refers to an activity of a crRNA-Cas13d complex binds to and optionally nicks an RNA which is not the target RNA. An off-target effect refers to binding of a crRNA-Cas13d complex with an RNA which is not the target RNA and any consequence(s) thereof, for example, reduction of a non-target RNA, reduction of a peptide or a protein encoded by the non-target RNA, increase or reduction of a peptide or a protein whose expression is regulated by the non-target RNA, and any physiological change(s) relating thereto.

In a further aspect, provided is a method for selecting a crRNA from two or more of crRNAs for directing a complex which comprises the crRNA and a CRISPR-associated protein 13d (Cas13d) or a variant thereof (i.e., Cas13d-crRNA complex or crRNA-Cas13d complex) to a target RNA. As disclosed herein, the crRNA is composed of a DR stem loop and a guide. The method comprises: determining on-target score of each of the two or more of crRNAs using the method as disclosed herein; and determining off-target score of each of the two or more of crRNAs using the method as disclosed herein. In a further embodiment, the method comprises selecting the crRNA with the highest on-target score and the lowest off-target score for directing the Cas13d-crRNA complex to the target RNA. In another embodiment, the method comprises constructing a model for incorporating the on-target score and the off-target score into one selection score via a modeling method. In one embodiment, a selection score equals an on-target score multiplied by a factor and minus the corresponding off-target score, wherein the factor can be any number (for example, an integer, a ratio, a rational number or an irrational number). In a further embodiment, the factor is a positive number. In one embodiment, the factor is any one of the following: 1, 2, 3, 4, 5, 6, 7, 8, 9, 10, 20, or 50. In one embodiment, the two or more of crRNA is capable of directing the Cas13d-crRNA to the same target RNA. In a further embodiment, the two or more of crRNA is capable of hybridizing to different (overlapping or nonoverlapping) hybridization regions of the same target RNA.

As used herein, a modeling method refers to a mathematical or statistical analysis, for example, random forest models, classification and regression tree models, boosting, Bayesian networks, Markov random field, linear and generalized linear models, boosted tree models, neural networks, support vector machines, general chi-squared automatic interaction detector models, interactive tree models, multiadaptive regression spline, machine learning classifiers, a multi hypothesis testing, a principal component analysis, and any combinations thereof. These statistical models are well known to those of skill in the art. Any other suitable algorithms in performing the characterization process. In variations, the analysis can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the analysis can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naive Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of analysis. The machine learning classifier may be a discriminant analysis (DA) machine learning classifier, a nearest neighbor (NN) machine learning classifier, a random forest (RF) machine learning classifier, or a support vector machine (SVM). A DA machine learning classifier may be a linear discriminant analysis (LDA) classifier, or a quadratic discriminant analysis (QDA) classifier. In one embodiment, the SVM classifier may have three kernels, including a linear kernel, a radial basis function (RBF) kernel, and a polynomial kernel. In another embodiment, the machine learning classifier may employ a convolutional neural network (CNN). In one embodiment, a modeling method may be performed on a computer.

As used herein, characterizing a feature or a grammatical variation thereof refers to a qualitative or quantitative manner of describing the feature. For example, it may be presence or absence of the feature, a numeric range of the feature, or a parameter/number/percentage calculated.

In certain embodiments, the ranking and/or any of the predicting methods as disclosed herein are determined in silicon in software. Such software is, for example, an R language program, a Python program or similar. Other codes performing the same function may also be used.

In another aspect, another method is described for screening or predicting on-target activity of a clustered regularly interspaced short palindromic repeats (CRISPR) RNA (crRNA), whereby the crRNA is capable of forming a complex with an RNA-targeting CRISPR-associated protein or a variant thereof and directing the complex to the target RNA. The method includes the step of (a) characterizing a plurality of crRNAs and their corresponding target by features comprising the presence of both a seed region located between guide RNA nucleotide bases 15 to 21 relative to the guide RNA 5′ end, characterized by a stabilizing, enriched sequence of G and C bases and an accessible target region characterized by an enriched sequence of A and U, surrounding the seed region on the 5′ end, 3′ end or both the 5′ and 3′ ends. In another embodiment an additional step (b) involves assessing on-target activity of each of the crRNAs of (a). In yet a further embodiment, an additional step (c) involves applying a machine learning model or deep learning model using the characterization of (a) and the on-target or off-target activity of (b). In one embodiment, input of the model comprises characterization(s) of the seed region and target regions of each crRNA and its corresponding target RNA, and output of the model is an on-target score of the crRNA, and wherein a higher score indicates a ranked on-target activity. In another embodiment the input and output can involve off-target scores. Still another step of the method includes (d) applying the model constructed in step (c) to a first crRNA and generating an on-target score or off-target score of the first crRNA.

The features of crRNA(s) and the corresponding target RNA(s) in step (a) are selected from any combination of at least the top 1, 2, 5, 10, 15, 20, 25, 30, 35 or more features of Table 5; any combination of 2 or more of the features of Table 5, at least the top 1, 2, 3, 4, 5, or 6 features of the RFGFP features listed in Table 2, at least the top 1, 2, 5, 10, 15, 20, 25, 30, or 33 features of the RFcombined features listed in Table 2; any combination of 2 or more features listed in Table 2 and having a DR sequence of Table 9.

In another embodiment, the method can include step (d) which further comprises applying the model constructed in step (c) to a second and further additional crRNA having the same target RNA, and generating an on-target score of the second crRNA.

In another embodiment, the on-target activity of step (b) is efficacy of the crRNA in forming a complex with a Cas13d protein or a variant thereof. In another embodiment, the on-target activity of step (b) is efficacy of the crRNA in hybridizing to the corresponding target RNA. In another embodiment, the on-target activity of step (b) is efficacy of the crRNA in directing a Cas13d-crRNA complex to the target RNA. In another embodiment, the on-target activity of step (b) is efficacy of the crRNA in reducing the corresponding target RNA after hybridizing to the target RNA. In another embodiment, the on-target activity of step (b) is enrichment or depletion of the CRISPR pooled screen readout. In another embodiment, the on-target activity of step (b) is efficacy of the guide of the crRNA or the target RNA after applying the crRNA and a Cas13d or a variant thereof to a cell or cell culture, a non-human organism or an in vitro, cell-free assay system. In another embodiment, the on-target activity of step (b) is the efficacy of the crRNA comprising guide sequences which mismatch the target, to allow the Class 2, Type VI effector protein to bind the target, but not elicit target degradation. In yet another embodiment, the method involves identifying on-target activity that includes binding without cleavage.

These methods can be applied to target RNA which is a messenger RNA (mRNA), a mature mRNA, a primary transcript mRNA (pre-mRNA), a ribosomal RNA (rRNA), a 5.8S rRNA, a 5S rRNA, a transfer RNA (tRNA), a transfer-messenger RNA (tmRNA), an enhancer RNA (eRNA), a small interfering RNA (siRNA), a microRNA (miRNA), a small nucleolar RNA (snoRNA), a Piwi-interacting RNA (piRNA), a tRNA-derived small RNA (tsRNA), a small rDNA-derived RNA (srRNA), a non-coding RNA (ncRNA), long (intergenic) non-coding RNA (lincRNA/lncRNA), a single-stranded RNA (ssRNA), a circular RNA (circRNA), a vault RNA (vRNA/vtRNA), a SmY RNA, a double-stranded RNA (dsRNA), a small Cajal body-specific RNA (scaRNA), an antisense RNA (aRNA/asRNA), a ribonuclease RNA (e.g. RNase P), a non-coding regulatory RNA (e.g. 7SK RNA), RNA-viruses, single stranded DNA, coding sequence (CDS) of a RNA, 5′ untranslated region (UTR) of a RNA, 3′ UTR of a RNA, or a intron of a RNA, or a satellite repeat sequence embedded in any of said RNA targets.

Yet other embodiments of the methods involve use of crRNA guides characterized by one or more of the DR sequences of Table 9. In other embodiments of the methods, the crRNA comprises a guide sequence which mismatches the target and allows the Class 2, Type VI effector protein to bind the target, but not elicit target degradation.

Also provided is a non-naturally occurring and/or synthesized and/or engineered crRNA ranked and selected by a method as disclosed herein.

C. crRNA

Also provided is a clustered regularly interspaced short palindromic repeat (CRISPR) RNA (crRNA) composed of a direct repeat (DR) stem loop sequence and a guide sequence, which is capable of hybridizing to a hybridization region of a target RNA. In one embodiment, the crRNA is a Class 2, Type VI crRNA which comprises a direct repeat (DR) stem loop sequence and a guide or spacer sequence. In another embodiment, the crRNA is characterized by having a DR sequence selected from one or more of the DR sequences of Table 9 above. In one embodiment the crRNA has a DR of SEQ ID NO: 2. In one embodiment the crRNA has a DR of SEQ ID NO: 14. In one embodiment the crRNA has a DR of SEQ ID NO: 25. In one embodiment the crRNA has a DR of SEQ ID NO: 36. In still other embodiments, the crRNA has a DR of any of SEQ ID NO: 1-46, or a variant thereof.

In one embodiment, the crRNA is non-naturally occurring. In another embodiment, the crRNA is synthesized. In another embodiment, the crRNA is an engineered sequence. The crRNA is capable of forming a complex with a Class 2, Type VI protein, such as Cas13d or a variant identified above. The crRNA is capable of directing the complex to the target RNA. In one embodiment, provided is a crRNA designed, generated, or selected by a method described herein.

In one embodiment, the crRNA does not require a protospacer in the target RNA for directing the complex. In a further embodiment, nt 15 to nt 21 of the crRNA matches with its corresponding hybridization “seed” region of the target RNA. In yet a further embodiment, one or two mismatches to the target RNA may be found outside of nt 15 to nt 21 of the crRNA. However, three or more mismatches are not allowed between the guide of the crRNA and its corresponding hybridization region of the target RNA.

Without wishing to be bound by the theory, the center of the nt 15 to nt 21 of the crRNA is theorized to coincide with conserved contacts between a helical domain in RfxCas13d protein and the backbone of the guide-target hybrid interface. This interaction resides opposite of the nt 17-18 of the guide within the target RNA. The helical domain is placed between both higher eukaryotes and prokaryotes nucleotide-binding (HEPN) domains needed for target cleavage, and mutation of the interacting amino acids in EsCas13d completely abolished target cleavage. See, Ref 28. Mismatches at around nt 18 of the crRNA may likely impair HEPN-domain activity.

In certain embodiments, the crRNA has one or more of the positively correlated features but not the negatively correlated features. In one embodiment, the features are listed in one or more of Tables 2 and 4-7 and FIGS. 6 and 13. In a further embodiment, ranges of the features are provided in Table 2. In one embodiment, the features are detailed in Table 7.

In certain embodiments, the crRNA having a DR stem loop which is about 30 nt long, for example, 29 nt, 30 nt, or 31 nt long. Based on its secondary structure, the DR stem loop is composed of, from the 5′ end to the 3′ end, a 5′ end, a stem loop which is capable of forming a self-hybridizing structure via paired nucleotides matching with each other, and a 3′ end. The 5′ and 3′ ends of the DR stem loop do not match to the target RNA or any nucleotide of the stem loop. In one embodiment, the stem loop comprises unpaired nucleotides. In a further embodiment, the middle 4 nucleotide residues of the stem loop are not paired and forming a loop. In yet a further embodiment, there is an additional two unpaired nucleotide residues in the stem loop forming a bulge. One example of loop and bulge can be seen in FIG. 8. In one embodiment, a crRNA comprises one unpaired nucleotide as the 5′ end of its DR stem loop.

In one embodiment, the crRNA has a stem loop with a motif selected from the following:
(I) 5′-(₁ (₂ (₃ (₄ (₅ (₆. (₇ (₈ (₉ . . . )₉ )₈ )₇. )₆ )₅ )₄ )₃ )₂ )₁-3′,
(II) 5′-. (₁ (₂ (₃ (₄ (₅. (₆ (₇ (₈ . . . )₈ )₇ )₆. )₅ )₄ )₃ )₂ )₁.-3′,
(III) 5′- . . . (₁ (₂ (₃ (₄. (₅ (₆ (₇ . . . )₇ )₆ )₅. )₄ )₃ )₂ )₁ . . . -3′,
(IV) 5′- . . . (₁ (₂ (₃. (₄ (₅ (₆ . . . )₆ )₅ )₄. )₃ )₂ )₁ . . . -3′, and
(V) 5′-(₁ (₂ (₃ (₄ (₅ (₆ (₇ (₈ (₉ (₁₀ . . . )₁₀ )₉ )₈ )₇ )₆ )₅ )₄ )₃ )₂ )₁- 3′,

wherein “(ₙ” and “)ₙ” represent a pair of nucleotides which matches with each other, and “.” represents an unpaired nucleotide in bulge or loop. As defined above, the self-hybridization stem loop of the DR stem loop starts from a nucleotide noted as “(₁” and ends at a nucleotide noted “)₁” in the motifs of (I) to (V). The “ . . . ” In the center of the motifs represent the unpaired nucleotides in a loop while the “.” flanked by “(” or “)” on both side represent the unpaired nucleotides in a bulge.

In one embodiment, the DR stem loop further contains 1 to 8 nucleotides at the 3′ end of the motif and preceding the guide. Additionally, or alternatively, the DR stem loop further contains a G residue at the 5′ end of the motif.

In certain embodiments, the DR stem loop comprises one of the following sequences SEQ ID NO: 1 to 13, or a modification thereof or the related sequences of Table 9, identified above:

(I)
AACCCCUACCAACUGGUCGGGGUUUGAAAC, SEQ ID NO: 1;
(II)
UACCCCUACCAACUGGUCGGGGUUUGAAAC, SEQ ID NO: 2;
(III)
UUCCCCUACCAACUGGUCGGGGUUUGAAAC, SEQ ID NO: 3;
(IV)
UUUCCCUACCAACUGGUCGGGGUUUGAAAC, SEQ ID NO: 4;
(V)
UUACCCUACCAACUGGUCGGGGUUUGAAAC, SEQ ID NO: 5;
(VI)
AACCCCGACCAACUGGUCGGGGUUUGAAAC, SEQ ID NO: 6;
(VII)
UACCCCGACCAACUGGUCGGGGUUUGAAAC, SEQ ID NO: 7;
(VIII)
UUCCCCGACCAACUGGUCGGGGUUUGAAAC, SEQ ID NO: 8;
(IX)
UUUCCCGACCAACUGGUCGGGGUUUGAAAC, SEQ ID NO: 9;
(X)
AACCCCUACCAACUGGUAGGGGUUUGAAAC, SEQ ID NO: 10;
(XI)
UACCCCUACCAACUGGUAGGGGUUUGAAAC, SEQ ID NO: 11;
(XII)
UUCCCCUACCAACUGGUAGGGGUUUGAAAC, SEQ ID NO: 12;
and
(XIII)
UUUCCCUACCAACUGGUAGGGGUUUGAAAC, SEQ ID NO: 13.

In yet a further embodiment, the DR stem loop is composed of a G-residue at the 5′ end followed by one of sequences (I) to (XIII) In certain embodiments, the crRNA does not have a G-quadruplex. In one embodiment, the presence or absence of a G-quadruplex is determined by RNAfold. In certain embodiments, each nt from nt −14 to nt −20 of the target RNA matches its corresponding region of the crRNA. In certain embodiments, the guide is about 23 nt long to about 33 nt long, or about 27 nt to about 30 nt long, or about 27 nt long, or about 23 nt long.

The efficacy of a crRNA in forming a complex with a Cas13d protein or a variant thereof and directing the complex to the target RNA may be measured. In one embodiment, the efficacy is at least about 10%, about 20%, about 30%, about 40%, about 50%, about 60%, about 70%, about 80%, about 90%, about 1 fold, about 1.5 fold, about 2 fold, about 3 fold, about 5 fold, about 10 fold higher than that of another crRNA. In another embodiment, at least about 10%, about 20%, about 30%, about 40%, about 50%, about 60%, about 70%, about 80%, about 90%, about 95%, or about 99% of the target RNA hybridized to the crRNA. In yet another embodiment, the amount of the target RNA is reduced for at least about 10%, about 20%, about 30%, about 40%, about 50%, about 60%, about 70%, about 80%, about 90%, about 95%, or about 99% after hybridization to the crRNA and being nicked by Cas13d or a variation thereof.

In another embodiment, the cRNA or nucleic acid molecule described herein comprises a guide sequence which mismatches the target and allows the Class 2, Type IV effector protein to bind the target, but not elicit target degradation.

### D. Other Components and Compositions

Also provided is a non-naturally occurring, synthesized (chemically or recombinantly) or engineered nucleic acid molecule comprising one or more of the crRNA(s) as disclosed, or a nucleic acid sequence complementary to the crRNA(s), or a nucleic acid sequence encoding the crRNA(s), or a nucleic acid sequence complementary to the crRNA coding sequence. In one embodiment, the nucleic acid molecule is a DNA.

In one embodiment, the nucleic acid molecule is a mature RNA. In one embodiment, the nucleic acid molecule comprises a DNA sequence encoding the crRNA(s). In a further embodiment, the nucleic acid molecule further comprises a first regulatory sequence directing expression of the crRNA(s). For example, the first regulatory sequence may comprise without limitation, a Pol III promoter, for example, a U6 promoter, a H1 promoter, a T7 promoter, and a 7SK promoter.

In another embodiment, the nucleic acid molecule further comprises a DNA sequence encoding a Class 2, Type VI effector protein or a variant thereof. In one embodiment, the encoded protein is any Class 2, Type VI protein. In a further embodiment, the protein is a Cas13d protein. In another embodiment, the effector protein is a RfxCas13d from Ruminococcus flavefaciens strain XPD3002. In another embodiment, other Cas13d proteins may be utilized, for example, an AdmCas13d from Anaerobic digester metagenome 15706, EsCas13d from Eubacterium siraeum DSM15702, P1E0Cas13d from Gut metagenome assembly P1E0-k21, UrCas13d from Uncultured Ruminoccocus sp., RffCas13d from Ruminoccocus flavefaciens FD1, and RaCas13d from Ruminoccocus albus. In a further embodiment, the feature(s), ranges of the features(s), and any combination thereof may be adjusted according to a Cas13d other than RfxCas13d.

In a further embodiment, the Cas13d or a variant thereof further comprises a nuclear localization signal (NLS) or a cytosolic signal or a nuclear-export signal (NES). In another embodiment the Cas13d or a variant thereof is fused to an endoplasmic reticulum localization element, an Outer Mitochondrial membrane localization element, a Mitochondria localizing element, a Nucleolus localizing element (NIK3x), a Nuclear lamina localizing element (LMNA) or a Nuclear pore complex localizing element (SENP2 ). In yet a further embodiment, the Cas13d or a variant thereof is capable of nicking a target RNA. In one embodiment, the Cas13d or a variant thereof has been engineered and does not have a nuclease activity, therefore referred to as a dead Cas13d.

In one embodiment, the DNA sequence encoding the effector, e.g., Cas13d, protein is under the control of a regulatory sequence directing expression thereof in a mammalian cell. In yet a further embodiment, the nucleic acid molecule comprises a second regulatory sequence which directs expression of the Cas13d protein or a variation thereof. In one embodiment, the second regulatory sequence comprises an RNA polymerase II (Pol II) promoter, for example, an EF-1 Alpha Short (EFS) promoter, or a Tet operator (tetO) promoter. In a further embodiment, the second regulatory sequence comprises one or more of the following: a polyadenylation (poly(A)) sequence, a selectable marker, a tag, and a Woodchuck Hepatitis Virus (WHP) Posttranscriptional Regulatory Element (WPRE) sequence. In certain embodiments, the tag is selected from one or more of the following: a FLAG tag, a poly(His) tag, a chitin binding protein (CBP) tag, a maltose binding protein (MBP) tag, a Strep tag, a glutathione-S-transferase (GST) tag, a thioredoxin (TRX) tag, a poly(NANP) tag, a V5 tag, a HA tag, a Spot tag, a T7 tag, a NE tag, a fluorescence tag, a Green Fluorescent Protein (GFP) tag, and a MYC tag. In one embodiment, the FLAG tag has a sequence of DYKDDDK, SEQ ID NO:47. In certain embodiments, the selectable marker is a puromycin resistance gene, a kanamycin resistance gene, a chloramphenicol resistance gene, a blasticidin S resistance gene, an ampicillin resistance gene, a tetracycline resistance gene, or a G418 resistance gene.

In certain embodiments, one nucleic molecule comprises the sequence for the crRNA and a separate nucleic molecule encodes the sequence of the Cas13d protein.

Also provided is a vector comprising a crRNA and or a nucleic acid molecule as disclosed. In one embodiment, the vector is a viral vector, a retrovirus vector, a lentiviral vector, an adenovirus vector an adeno-associated virus vector, or a hybrid viral vector. In another embodiment, the vector is a non-viral vector or an analogous carrier, such as a nanoparticle, a lipid complex, a polymer, a quantum dot, a carbon nanotube, a magnetic nanoparticle, or a gold nanoparticle. Further, a vector (for example, a plasmid) for producing of the vector is provided.

In still another embodiment, a ribonucleoprotein (RNP) complex as described herein includes a Class 2, Type VI effector protein and a crRNA, as defined herein. In still another embodiment, a cell is provided which contains one or more of the cRNA, nucleic acid molecules, RNP or compositions described herein. The cell may be mammalian, preferably a human cell. In other embodiments, the cell may be bacterial.

Additionally, provided is a library comprising a plurality of crRNAs or nucleic acid molecules or RNPs or vectors or cells as disclosed. In one embodiment, each of the crRNA is capable of directing a Cas13d or a variant thereof to a different target RNA or a different region of one target RNA. In another embodiment, the library is a lentiviral library.

A composition is also provided comprising a pharmaceutical acceptable carrier and one or more crRNA(s), RNPs, or nucleic acid molecule(s) or vector(s), or cells as disclosed. These compositions may be for pharmaceutical use and thus useful in the treatment of a disease associated with an abnormal RNA or misregulation of an RNA transcript. Some examples of these diseases are the diseases mentioned specifically above.

In yet a further embodiment, the crRNA, RNPs, pharmaceutical compositions, cells, vectors and libraries may also comprise crRNA having guide sequences which mismatch the target and allow the Class 2, Type VI effector protein to bind the target, but not elicit target degradation when used in the methods known to those of skill in the art as well as the methods described and exemplified specifically herein.

### E. Methods of Use

One or more of the crRNAs, nucleic acid molecules, RNPs, vectors, cells, and libraries described herein are useful in a variety of methods including without limitation, treating a disease associated with an abnormal RNA; screening functional RNA(s); knocking-down, detecting, or editing a target RNA; or detecting or editing splicing, alternative isoforms, intron retention or differential UTR usage, or binding but not degrading the target.

In one aspect, the crRNA(s), nucleic acid molecule(s), RNB(s), vector(s), cell(s), or composition(s) containing one of more of them are used as a medicament, for example, in the treatment of a disease associated with an abnormal RNA such as by reducing the level of the abnormal RNA. Such disease may be a cancer/tumor, a virus infection, or a genetic disorder. In one embodiment, the treatment comprises contacting a target cell, and/or a biological sample from a subject having or suspected of having the disease with the crRNA(s), nucleic acid molecule(s), RNB(s), or vector(s) described herein. In further embodiment, target RNA of the crRNA(s) is/are the abnormal RNA(s) associated with the disease. In yet a further embodiment, the level of the abnormal RNA(s) in the target cell and/or in the biological sample is reduced. In one embodiment, the level of the abnormal RNA(s) after the treatment is reduced to at least about 10%, about 20%, about 30%, about 40%, about 50%, about 60%, about 70%, about 80%, about 90%, or about 95% of the level before the treatment or the level of a subject having this disease. In another embodiment, the level of the abnormal RNA(s) after the treatment is about 10%, about 20%, about 30%, about 40%, about 50%, about 60%, about 70%, about 80%, about 90%, about 100%, about 1.2 fold, about 1.5 fold, about 2 fold, about 3 fold or about 4 fold of a control level of a subject who is free of the disease. In still other methods, the targets are blocked but not degraded. In still other embodiments, the targets are modified temporarily. In other embodiment, the targets are modified permanently.

In one embodiment, a method of treating a disease associated with an abnormal RNA or misregulation of an RNA transcript, comprises administering to a subject in need thereof the crRNA, nucleic acid molecules, vectors, RNBs, cells, or pharmaceutical compositions described herein. The administering step involves in one embodiment, delivering the selected or designed crRNA as a mature RNA to a cell that expresses an RNA-targeting CRISPR-associated protein, e.g., a Class 2, Type VI protein, such as Cas13d or a variant. In one embodiment the cell has been conditioned or modified to express the Cas13d or variant, and the administering occurs ex vivo. In another embodiment, the administering step involves delivering the crRNA described herein in a vector which co-expresses the RNA-targeting CRISPR-associated protein. In still another embodiment, the administering step involves delivering the crRNA and RNA-targeting CRISPR-associated protein as a ribonucleoprotein complex to the subject. In still a further embodiment, the administering step involves delivering the nucleotide molecule containing the crRNA with a separate nucleotide molecule that expresses the RNA-targeting CRISPR-associated protein.

As used herein, the terms “cancer” and “tumor” are used interchangeably and refer to an abnormal cell growth invading or spreading to other parts of the subject or having a potential of the invading or spreading. In order to achieve the abnormal cell growth, to invade other parts of the subject, and/or to evade an immune reaction, abnormal RNAs may be present in a tumor/cancer cell. The cancer/tumor includes, but is not limited to, a solid tumor (e.g., breast, colon, ovarian, lung, liver and glioma, Mesothelioma, and non-small cell lung cancer), a B cell lymphoma, a Cutaneous T cell lymphoma and a Lymphoid leukemia.

Upon virus infection, a target cell may generate abnormal RNA(s) in order to neutralize the virus. Additionally or alternatively, after the virus' entry to a target cell, the virus may utilize the RNA producing machinery of the target cell producing abnormal RNA(s) in order to replicate the virus, or to lyse the target cell, or to perform other function(s) required by fulfilling the virus life cycle. Such virus infection may include HCV infection and related liver diseases, smallpox, the common cold and different types of flu, corona virus infections, measles, mumps, rubella, chicken pox, and shingles, hepatitis (HCV, HBV, or HAV), HIV, herpes and cold sores, polio, rabies, Ebola and Hanta fever.

Abnormal RNA(s) may also be found in other diseases, including, without limitation, Atherosclerosis, Polycystic Kidney Disease, Cardiac disease, Cardiac stress, Myocardial infarction, Kidney fibrosis, Cardiac fibrosis, diabetes, Diabetes-related kidney complications, type 2 diabetes, non-alcoholic fatty liver diseases, mycosis fungoides, and Scleroderma. Other representative examples of disease-causing defects associated with misregulation or defects in RNA include without limitation Prader Willi syndrome, Spinal muscular atrophy (SMA), Dyskeratosis congenita (X-linked), Dyskeratosis congenita (autosomal dominant), Dyskeratosis congenita (autosomal dominant), Diamond-Blackfan anemia, Shwachman-Diamond syndrome, Treacher-Collins syndrome, Prostate cancer, Myotonic dystrophy, type 1 (DM1 ), Myotonic dystrophy, type 2 (DM2 ), Spinocerebellar ataxia 8 (SCA8 ), Huntington's disease-like 2 (HDL2 ), Fragile X-associated tremor ataxia syndrome (FXTAS), Fragile X syndrome, X-linked mental retardation, Oculopharyngeal muscular dystrophy (OPMD), Human pigmentary genodermatosis, Retinitis pigmentosa, Cartilage-hair hypoplasia (recessive), Autism, Beckwith-Wiedemann syndrome (BWS), Charcot-Marie-Tooth (CMT) Disease, Charcot-Marie-Tooth (CMT) Disease, Amyotrophic lateral sclerosis (ALS), Leukoencephalopathy with vanishing white matter, Wolcott-Rallison syndrome, Mitochondrial myopathy and sideroblastic anemia (MLASA), Encephalomyopathy and hypertrophic cardiomyopathy, Hereditary spastic paraplegia, Leukoencephalopathy 2, susceptibility to diabetes, deafness, and MELAS syndrome. See, for example, Thomas A. Cooper, et al, RNA and Disease, Cell, 136 (4 ): 2009, 777-793, ISSN 0092-8674; Scotti, M., Swanson, M. RNA mis-splicing in disease. Nat Rev Genet 17, 19-32 (2016 ). https://doi.org/10.1038/nrg.2015.3; Matsui, M., Corey, D. Non-coding RNAs as drug targets. Nat Rev Drug Discov 16, 167---179 (2017 ). https://doi.org/10.1038/nrd.2016.117; Rupaimoole, R., Slack, F. MicroRNA therapeutics: towards a new era for the management of cancer and other diseases. Nat Rev Drug Discov 16, 203-222 (2017 ). https://doi.org./10.1038/nrd.2016.246; and Rohilla, K. J., Gagnon, K. T. RNA biology of disease-associated microsatellite repeat expansions. Acta Neuropathol Commun 5, 63 (2017 ). https://doi.org/10.1186/s40478-017-0468-y, incorporated by reference herein.

In certain embodiments, the abnormal RNA(s) is/are presented in a biological sample. In a further embodiment, the abnormal RNA(s) may not be within a cell.

In another aspect, a functional screening method is provided. The method comprises contacting one or more crRNA(s), and/or nucleic acid molecule(s), and/or vector(s), and/or a library as disclosed with a target cell of a cell culture, a tissue, or a subject. In one embodiment, the method comprises amplifying the nucleic acid molecule or the vector in the target cell, and optionally quantifying the nucleic acid molecule or the vector.

In one embodiment, a Cas13d protein is expressed by a nucleic acid molecule or a vector in the target cell. Thus in the target cell, the crRNA forms a complex with a Cas13d or a variation thereof, and directs the complex to a target RNA. In a further embodiment, the nucleic acid molecule or vector is the same nucleic acid molecule or vector which comprises or expresses the crRNA(s). In another embodiment, the nucleic acid molecule or vector expresses the Cas13d protein but not the crRNAs and thus, is referred to as “Cas13d molecule” or “Cas13d vector” as used herein. In one embodiment, the ratio of the Cas13d molecule (or Cas13d vector) to a crRNA (or nucleic acid molecule and/or vectors providing the crRNA) is about 100 to 1 to about 1 to 100, including each ratio therebetween. In one embodiment, the ratio is about 10 to 1, about 5 to 1, about 4 to 1, about 3 to 1, about 2 to 1, about 1 to 1, about 1 to 2, about 1 to 3, about 1 to 4, about 1 to 5, or about 1 to 10. In a further embodiment, the ratio is a molar ratio.

In one embodiment, the encoded Cas13d protein is a RfxCas13d from Ruminococcus flavefaciens strain XPD3002. Other Cas13d may also be utilized, for example, AdmCas13d from Anaerobic digester metagenome 15706, EsCas13d from Eubacterium siraeum DSM15702, P1E0Cas13d from Gut metagenome assembly P1 E0-k21, UrCas13d from Uncultured Ruminoccocus sp., RffCas13d from Ruminoccocus flavefaciens FD1, and RaCas13d from Ruminoccocus albus. In a further embodiment, the Cas13d or a variant thereof further comprises a nuclear localization signal (NLS) or a cytosolic signal or a nuclear-export signal (NES). In yet a further embodiment, the Cas13d or a variant thereof is capable of nicking a target RNA. In one embodiment, the Cas13d or a variant thereof has been engineered and does not have a nuclease activity. In one embodiment, the Cas13d is conjugated to a reporter molecule.

In one embodiment, the method reduces level of one or more of target RNA(s) in a target cell. In a further embodiment, the method functionally knocks down or knocks out one or more gene(s) expressing the target RNA(s). In yet a further embodiment, the method knocks down or knocks out one or more gene(s) in a plurality of targets cells in parallel.

In certain embodiments, a selective pressure or a stimulus is applied to the target cells prior to, during or after the contacting step, which is referred to as a perturbation step. Such selective pressure or a stimulus includes, for example, a chemical agent or a biological agent or actively physically disturbing the target cell(s).

The term chemical agent includes various small molecule drugs/compounds, while the term biological agent refers to biological drugs, which are a diverse category of drugs and are generally large, complex molecules. These biological drugs may be produced through biotechnology in a living system, such as a microorganism, plant cell, or animal cell. Types of biological products approved for use in the United States, including therapeutic proteins (such as filgrastim), monoclonal antibodies (such as adalimumab), vaccines (such as those for influenza and tetanus), cell therapy drug (for example, CarT), and gene therapy drug (for example, recombinant AAV vectors). During the perturbation step, the cells may be incubated with the chemical and/or biological agent or any combinations thereof, such as a library of peptides or a library of small molecules or a library of anti-cancer drugs, which are available commercially or publicly. See, for example, www.selleckchem.com/screening/anti-cancer-compound-library.html?gclid=CjwKCAjw0tHoBRBhEiwAvP1GFfLrUWZGJpXyE_QMr_f3NMvn9tC8433K8edIeOYkL08wUNdHzzwgFhoCquQQAvD_BwE, www.genscript.com/peptide-library.html, www.creative-biolabs.com/drug-discovery/therapeutics/whole-peptide-library.htm, phoenixpeptide.com/products/category/Peptide-Libraries/, www.selleckchem.com/screening/express-pick-library-premium-version.html?gclid=CjwKCAjw0tHoBRBhEiwAvP1GFTm7F6ezXNk1pUNajAWqP8Nc4COj2N1MNTes9pEGADe8nMF7UmUgPxoCT9cQAvD_BwE, www.selleckchem.com/screening/fda-approved-drug-library.html and www.chembridge.com/screening_libraries/. In certain embodiments, the cells are contacted with various chemical drugs or biological drugs for large-scale drug screens. In certain embodiments, the cells are treated via CRISPR-Cas enzyme and various guide RNA. The term physical disturbance refers to an active mixing, shaking, stretching, or stirring of the target cell(s). In certain embodiments, a population of cells is treated separately with any one of the perturbations as described herein or with any combinations of the perturbations, resulting in a heterologous population of cells.

In certain embodiments, the method further comprises assessing cell viability, cell proliferation, cell apoptosis, cell death, cell phenotype, existence or concentration of a molecule (for example, the target RNA(s)), protein or cell marker expression, or response to a stimulus of a target cell, or a function which may be achieved by the cell culture, tissue, or subject comprising the target cell(s).

In a further aspect, provided is a method for detecting a target RNA. In one embodiment, the target RNA is an abnormal RNA associated with a disease. Suitable diseases have been discussed in the earlier sections. In a further embodiment, the target RNA is a virus RNA.

The method comprises contacting a biological sample with a crRNA (or a nucleic acid or a vector expressing the crRNA) as disclosed. In one embodiment, the crRNA is conjugated with a reporter molecule. In a further embodiment, the crRNA hybridizes to a mock RNA which is conjugated to a reporter molecule, whereby during the contacting step, the target RNA competitively hybridizes to the crRNA thus releasing the mock RNA with the reporter molecule. In another embodiment, the method further comprises contacting the biological sample with a Cas13d or a variant thereof, prior to, concurrently with, or after the contacting step with the crRNA(s). In a further embodiment, the Cas13d or a variant thereof is expressed by a nucleic acid molecule or a vector as described herein (which may be the same nucleic acid molecule or vector providing a crRNA or a different one) in a target cell of the biological sample. In yet a further embodiment, the Cas13d or a variant thereof comprises (for example, via conjugation to) a reporter molecule. In certain embodiments, the method comprises detecting the presence or the level of a reporter molecule, which is an indication of presence or the level of the abnormal RNA in the biological sample.

In certain embodiments, the abnormal RNA(s) is/are presented in a biological sample. In one embodiment, the abnormal RNA(s) is in a target cell of the biological sample. In another embodiment, the abnormal RNA(s) may not be within a cell. In a further embodiment, the abnormal RNA(s) may be released from a target cell before the contacting step.

In yet a further aspect, a method for editing or modifying a target RNA is provided, comprising contacting a crRNA-Cas13d RNB complex with a target RNA. In one embodiment, this method or any composition used in the method is used for treatment of a disease associated with the target RNA.

In one embodiment, the crRNA of the complex is as disclosed herein. In a further embodiment, the complex is produced by a vector or a nucleic acid sequence disclosed. In one embodiment, the Cas13d nicks the target RNA. In another embodiment, the Cas13d has been engineered to have no nuclease activity. Other suitable Cas13d variants have been discussed in other sections of this application.

In a further embodiment, the Cas13d of the complex is engineered to edit or modify an RNA, for example. For example, the Cas13d may be conjugated to an RNA aminase, deaminase (e.g., ADAR, ADAR1, ADAR2 ), methylase, or demethylase (e.g., ALKBH5 ). In another embodiment, the Cas13d is conjugated to a splicing factor, for example a RBFOX1 or RBM38, whereby exon inclusion in the target RNA is induced when the hybridization region is at the downstream intron (i.e., intron at the 3′ side of an exon), and whereby exon exclusion in the target RNA is induced when the hybridization region is within the target exon. In yet another embodiment, the Cas13d is conjugated to a polyadenylation factor, for example, Nudix hydrolase 21 (NUDT21 ), whereby polyadenylation of RNA is induced at the hybridization region of the target protein.

In still another aspect, a method is provided for improving the efficiency of targeting or stabilization of a Class 2, Type VI crRNA which comprises a direct repeat (DR) stem loop and a guide or spacer sequence. Such a method involves replacing the DR stem loop sequence of a crRNA which targets inefficiently with a DR sequence selected from one or more of the DR sequences of SEQ ID Nos: 1 to 46 of Table 9, or a modification thereof.

In yet another embodiment, a method is provided that can use active Class 2, Type IV enzymes for cleaving a primary target, while using the same enzyme to block another secondary target without cleaving it. Similarly, the method can block multiple targets without cleaving the targets. In one embodiment, the primary target is a disease-causing or disease-related target and a secondary target is an interfering, e.g., RNA regulatory element(s). The secondary target can be blocked without degradation. It has been observed that Cas13a target RNA binding affinity and HEPN-nuclease activity are differentially affected by the number and the position of mismatches between the guide and the target. See, e.g., Tambe, A et al., 2018 July, RNA Binding and HEPN-Nuclease Activation Are Decoupled in CRISPR-Cas13a, Cell Repts., 24:1025-1036, incorporated by reference herein. Guide RNA and target interaction is needed at the seed region to elicit nuclease function and target degradation. Therefore, mismatches at the seed region of about 4 or more nucleotide bases still lead to pronounced binding but without nuclease activation. This is likely a conserved feature between many Cas13 proteins, which all have an extended RNA-RNA interaction interface, which is long enough for strong binding to the target site.

This method of blocking RNA targets without degradation in one embodiment involves administering to a cell expressing an RNA-targeting CRISPR-associated protein or to a subject crRNAs capable of forming a complex with the RNA-targeting CRISPR-associated protein or a variant thereof and directing the complex to the target RNA, wherein said crRNAs comprise a DR sequence and a guide or spacer sequences. The guide or spacer sequence of the crRNAs are characterized by forming extended mismatches to the target site in the seed region. In one embodiment, the crRNA has a guide sequence with 4 or more mismatches in the seed region located between guide RNA nucleotide bases 15 to 21 relative to the guide RNA 5′ end. In another embodiment, the crRNA and target are characterized by a stabilizing, enriched sequence of G and C bases and an accessible target region characterized by an enriched sequence of A and U, surrounding the seed region on the 5′ end, 3′ end or both the 5′ and 3′ ends. In still another embodiment, the DR sequence of the crRNA having the mismatched sequence is one of the DR sequences of Table 9. In yet a further embodiment, the crRNAs are designed and selected by use of the scoring methods described herein.

Because this method can be used to block RNA regulatory elements without degradation of the target by using guide crRNAs with extended mismatches to the target site in the seed regions, the method can be extended to alternate targets that require blocking. In one embodiment, this method can be employed to permit Cas13d (or another Class 2, Type VI protein), to bind and mask/block a binding sites for another RNA binding protein. In one embodiment, a single nucleotide polymorphism may lead to a unwanted binding site that is not desired. The use of a mismatched crRNA can block that unwanted site using active Cas13d instead of inactive Cas13d. Thus, in one embodiment of a method for treating or modifying a disease target, more than one function with active Cas13 can be accomplished. In one embodiment of a method to treat disease or modify genes/proteins causing disease, employs a step of administering a perfect match guide to destabilize a first target RNA directly related to disease. Simultaneously or before treatment with the perfect match crRNA, a step of administering a mismatched crRNA with active Cas13d (via mature RNA, the nucleic acid molecules expressing the crRNA and encoding the Cas13d, or delivering separate molecules or vectors, or delivering the RNP complex) to the same cell to block a another non-desired site, e.g., a regulatory site, without destabilizing the first target.

In yet another embodiment, the method employs the desired effector protein (e.g., active Cas13d) within the same cell to degrade a target RNA based on perfect matching, and protect another target RNA by binding and blocking a target site, such as a cis regulatory element that can serve as a binding site for another RNA-binding protein (RBP).

Even a single mismatch at the center of the seed region (e.g., position 18 relative to the 5′ end of the guide RNA) can lead to partial or complete loss of target cleavage. This is presented in FIG. 1h. This feature can be used to discriminate closely related transcript isoforms that differ in only a single mismatch. Such a scenario can be present in monoallelic single nucleotide variants (SNV or SNP) where one allele expresses a “healthy” transcript isoform, and the other allele carries an malignant variant.

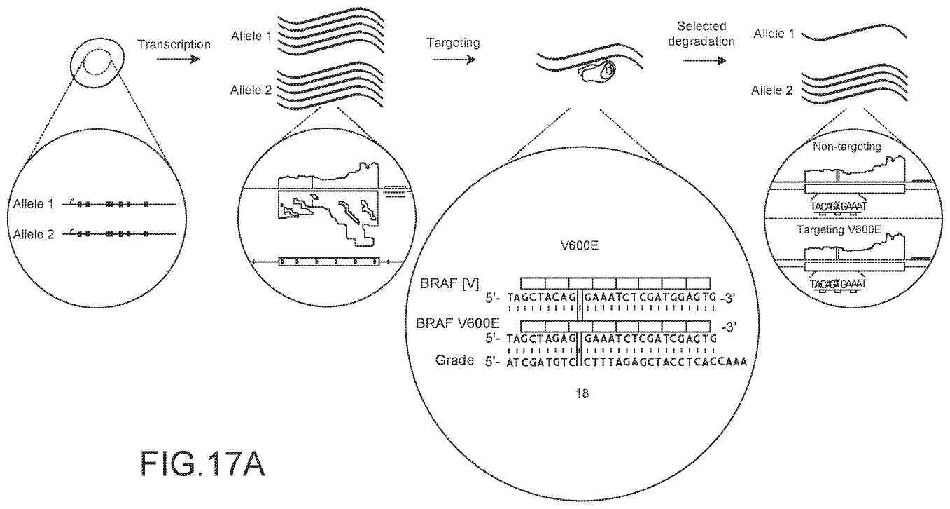

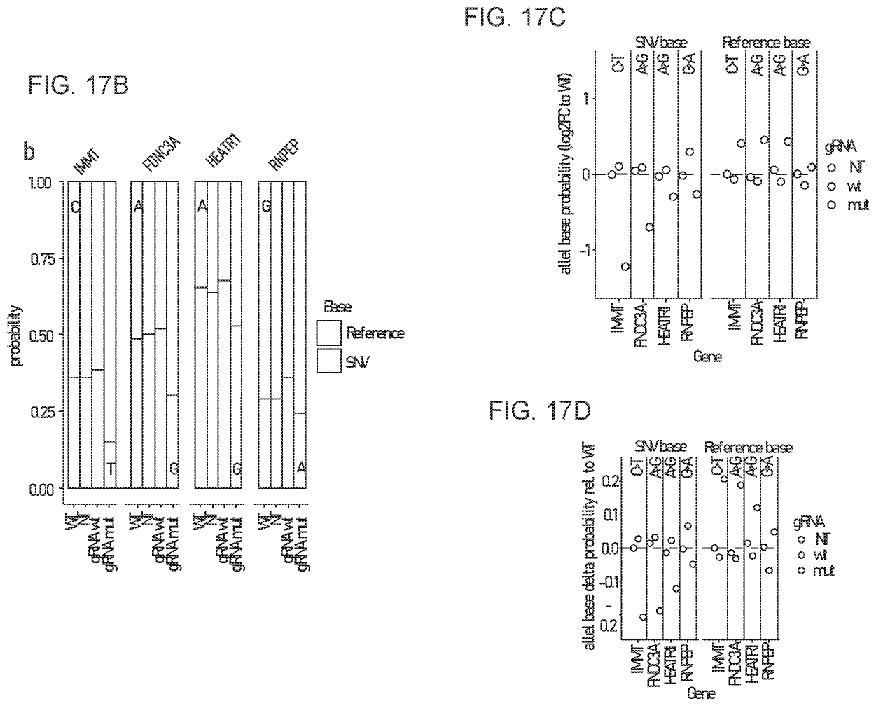

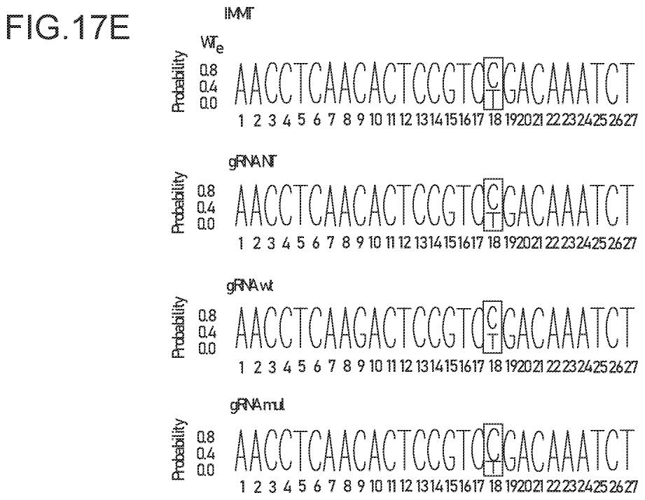

FIGS. 17a-e demonstrates this method with the example of the V600E mutation in the BRAF gene. We selected predicted malignant SNVs that are present in the COSMIC database (see, e.g., cancer.sanger.ac.uk/cosmic) and present as a monoallelic SNP in HEK293 cells. We used Cas13 to target either the reference isoform (gRNA wt) or the malignant isoform (gRNA mut) and compared the frequency of observed variant relative to the wildtype state, or the state when we used a random non-targeting (NT) guide RNA. We found that for four genes, we shifted the proportion of malignant isoform specifically if we target the malignant isoform. FIG. 17a provides the general overview of this approach. FIGS. 17b to 17e present different visualization of SNV specific targeting for four genes with predicted malignant outcome. The SNV base changes with a log 2 fold change relative to the abundance in the wild type state specifically when the SNV carrying transcript is targeted (gRNA mut; red dot). FIG. 17d shows the same data but quantifies the delta/difference in the base probability. Finally, FIG. 17e shows the example of the IMMT gene data and how the observed base probabilities change presented as a average sequence motif.

### EXAMPLES

The following examples disclose both general and specific embodiments of the disclosed compositions and methods described herein, which should be construed to encompass any and all variations that become evident as a result of the teaching provided herein.

### Example 1: GFP Model

Experiments were conducted with respect to in vivo RfxCas13d transcript tiling and permutation screen in mammalian cells. We also evaluated crRNA and target site features for RNA knock-down efficacy using machine learning approaches and have developed an algorithm and easy-to-use webtool for the optimal design of RfxCas13d guides. Using this algorithm, we provided guide predictions for all protein coding transcripts in a human subject. Moreover, we identified a critical seed region within the RfxCas13d guide sequence. This region allows for the discrimination closely related target transcripts by a single nucleotide distance. We showed, that this target sensitivity can be leveraged in vivo for allelic discrimination.

Via these experiments, it was confirmed that RfxCas13d provides robust target RNA knock-down outperforming two other recently identified type VI-B CRISPR proteins PguCas13b and PspCas13b. Nuclear localization/export-tagged nucleases, variable guide lengths, and mutations of the direct repeat were compared in order to develop an optimized RfxCas13d platform.

Previous work on Cas13d did not identify the existence of a critical seed region. Here we showed that a single mismatch between guide and RNA target site within the seed region (nucleotides 15-21 ) can largely disrupt target knock-down. We show that this feature can be used to discriminate closely related RNA species, such as allele-specific single nucleotide polymorphisms—demonstrating a new application of RNA-targeting CRISPR enzymes in vivo.

We systematically evaluated guide RNA and target RNA sequence, secondary structure and hybridization features for perfect match guide RNAs. We show that the crRNA accessibility has a profound impact on target knock-down efficacy, while target site accessibility at least in the GFP transcript has neglectable predictive value.

Using a set of guide RNA and target RNA features, we built an on-target model to predict crRNAs with high knock-down efficacy. We showed that our model performs better on our screen test data than previous models developed for Cas9 nuclease guide design. Specifically, our model is able to explain 37% of the variance in our screen data, while a widely-used Cas9 guide model explains only 21% of the screen variance (Doench et al., Nature Biotechnology, 2016 ).

We confirmed generalizability of our guide design by testing 12 crRNAs on two endogenous genes and show that in 10 out of 12 cases we were able to correctly predict lower or higher guide efficacies.

The largest RNA-targeting screen in mammalian cells to date was performed. In total we gathered information for ˜7,000 RNA-targeting guides, with more than 6,500 guide permutations. This increases the number of data points from previous studies in mammalian cells by more than two orders of magnitude.

We developed a simple algorithm that allows the user to predict guide efficacies on target RNAs. We applied this model to all protein coding transcripts in the human genome and provide a resource for the scientific community for optimal guides. We also created an online, web-based repository that allows the user to select a target mRNA based on cell-type specific isoform expression levels and to visually explore predicted guide scores across target mRNAs (similar to what we previously designed for Cas9, c.f. Meier et al., Nature Methods, 2017 ).

Previous studies hypothesized that “anti-tag” sequences (for self vs. non-self-discrimination in bacteria) would likely be found in Cas13d. Here, we definitively demonstrate the lack of a similar anti-tag for RfxCas13d, confirming the absence of restrictive protospacer flanking sequences.

All of this together defines a state-of-the-art approach to derive both a comprehensive evaluation of RfxCas13d guide design rules as well as a needed model of effective RfxCas13d guides. RNA-targeting Cas proteins have a similarly large impact in molecular biology and medical application; thus, accurate guide prediction is of immense value for this newly developing field.

### Example 2: GFP and Combined Models

We tested 21,763 additional guide RNAs and added 5 new pooled CRISPR-Cas13 screens. We used the data to train an improved on-target guide prediction model and show that our model is generalizable across a large number of endogenous genes.

To validate our initial GFP-screen-based guide prediction model, we conducted 2 pooled Cas13d fitness screens targeting 45 essential genes and 65 control genes with a total 4,803 guide RNAs. In these screens, we confirm that predicted high-scoring guide RNAs show better target knockdown compared to low-scoring guide RNAs for the majority of essential genes.

We show that guide RNA depletion in pooled CRISPR-Cas13d fitness screens is specific to essential genes and that gene-level guide depletion scores are in agreement with RNAi-based and CRISPR-Cas9 derived gene essentiality scores. Through this comparison with other functional genomics methods, we show that Cas13d can be utilized for transcriptome-wide forward genetic screens.

We conducted 3 additional tiling screens on endogenous target genes (3 cell surface receptors) with a total of 16,960 additional guide RNAs. We demonstrate efficient targeting of endogenous human transcripts and confirm that mismatches in the critical seed region (discovered in our initial GFP screen) also reduce targeting of endogenous transcripts.

We target complex features of endogenous transcripts beyond coding regions. Through these experiments, we show that Cas13 targeting is most effective in CDS regions, then 5′/3′ UTRs, and least effective in introns. We also show evidence that Cas13 competes with splicing factors in intronic sequences and with the exon-junction-complex. These insights were not possible from the original GFP screen in our initial submission, as the transgene did not contain introns or UTRs. These results demonstrate new applications of Cas13 pooled screens for the study of splicing, alternative isoforms, intron retention and differential UTR usage.

We updated our Cas13d guide design model learning features across all 4 tilling screen datasets. We evaluated the generalizability of both our initial model as well as the updated model on 48 endogenous genes. Importantly, we show that our updated model shows improved prediction accuracy compared to the model in our initial submission. The improved model explains 47% of the variance in the data set with an average Spearman correlation to the held-out data of 0.67.

Code and software are generated to reproduce our entire analyses (data not shown). Moreover, we greatly improved the utility and performance of our web-based repository for guide RNA predictions targeting all protein-coding transcripts in the human genome (cas13design.nygenome.org).

### Example 3: Methods

A. Cloning of Cas13 Nuclease, Guide RNAs and Destabilized EGFP Plasmids

Using Gibson cloning, we modified the EF1a-short (EFS) promoter-driven lentiCRISPRv2 (Addgene 52961 ) or lentiCas9-Blast (Addgene 52962 ) plasmids with several different transgenes¹. For the destabilized EGFP construct, we introduced a PEST sequence and nuclear localization tag on EGFP to create EFS-EGFPd2PEST-2A-Hygro from lentiCas9-Blast. To test the upstream U-content, we introduced a multiple cloning site (MCS) into EFS-EGFPd2PEST-2A-Hygro right after the stop codon, and used the MCS to introduce oligonucleotide sequences with variable U-content¹.

For the CRISPR Type-VI orthologs, we cloned effector proteins (PguCas13b: Addgene 103861, PspCas13b: Addgene 103862, RfxCas13d: Addgene 109049 ) and their direct repeat (DR) sequences (PguCas13b: Addgene 103853, PspCas13b: Addgene 103854, RfxCas13d: Addgene 109053 ) into lentiCRISPRv2. In this manner, we created lentiRNACRISPR constructs: hU6-[Cas13 DR]-EFS-[Cas13 ortholog]-[NLS/NES]-2A-Puro-WPRE, where [Cas13 ortholog] was one of PguCas13b, PspCas13b, or RfxCas13d and [NLS/NES] was either a nuclear localization signal or nuclear export signal. To generate doxycycline-inducible Cas13d cell lines, we cloned NLS-RfxCas13d-NLS (Addgene 109049 ) into TetO-[Cas13]-WPRE-EFS-rtTA3-2A-Blast. For the screens, we changed the DR in the lentiGuide-Puro vector (Addgene 52963 ) to contain the RfxCas13d DR using Gibson cloning to create lentiRfxGuide-Puro¹. All plasmids will be made available on Addgene.

Guide cloning was done as described previously¹. All constructs were confirmed by Sanger sequencing. All primers used for molecular cloning and guide sequences are not shown.

B. Cell Culture and Monoclonal Cell Line Generation

HEK293FT cells were acquired from Thermo Fisher Scientific (R70007 ) and A375 cells were acquired from ATCC (CRL-1619 ). HEK293FT and A375 cells were maintained at 37° C. with 5% CO₂ in D10 media: DMEM with high glucose and stabilized L-glutamine (Caisson DML23 ) supplemented with 10% fetal bovine serum (Serum Plus II Sigma-Aldrich 14009C) and no antibiotics.

To generate doxycycline-inducible RfxCas13d-NLS HEK293FT and A375 cells, we transduced cells with a RfxCas13d-expressing lentivirus at low MOI (<0.1 ) and selected with 5 μg/mL Blasticidin S (ThermoFisher A1113903 ). Single cell colonies were picked after by sparse plating. Clones were screened for Cas13d expression by western blot using mouse anti-FLAG M2 antibody (Sigma F1804 ).

For the GFP tiling screen RfxCas13d-expressing cells were transduced with EFS-EGFPd2PEST-2A-Hygro lentivirus at low MOI (<0.1 ) and selected with 100 μg/ml Hygromycin B (ThermoFisher 10687010 ) for 2 days. Single-cell colonies were grown by sparse plating. Resistant and GFP-positive clonal cells were expanded and screened for homogenous GFP expression by FACS.

C. Transfection and Flow Cytometry

For all transfection experiments, we seeded 2×10⁵ HEK293FT cells per well of a 24-well plate prior to transfection (12-18 hours) and used 500 or 750 ng plasmid together with a 5-to-1 ratio of Lipofectamine 2000 (ThermoFisher 11668019 ) or 1 mg/mL polyethylenimine (Polysciences 23966 ) to DNA (e.g. 2.5 μl Lipofectamine2000 or PEI mixed with 0.5 μg plasmid DNA). Flow cytometry or fluorescence-assisted cell sorting (FACS) was performed at 48 hrs post-transfection. All transfection experiments were performed in biological triplicate.

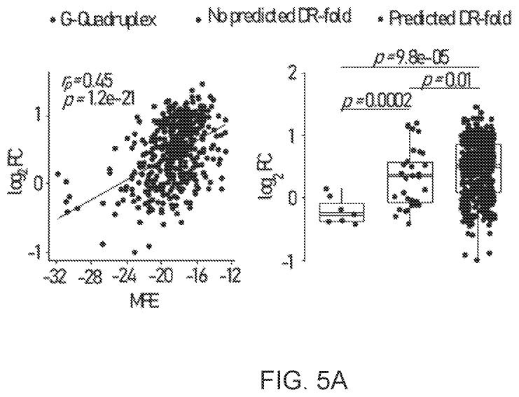

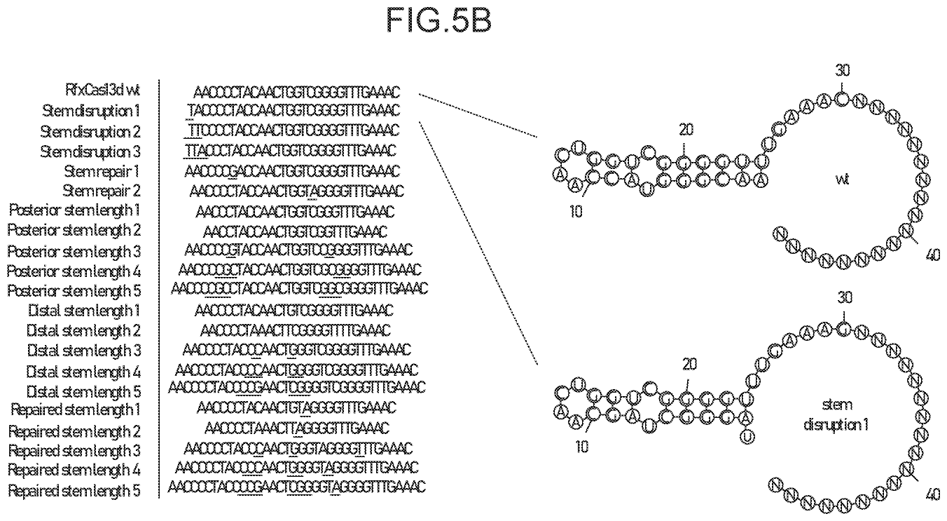

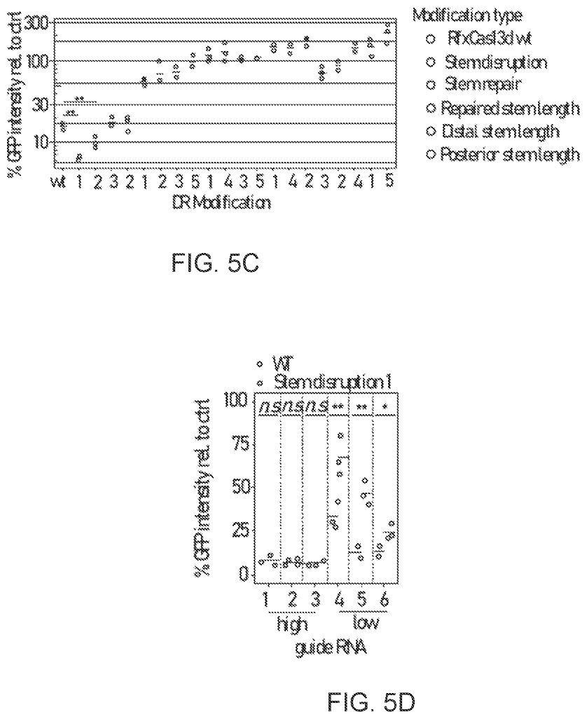

We compared Type IV CRISPR Cas protein knock-down efficacy. Lentiviral vectors for combined CRISPR Type IV enzyme and crRNA delivery were created. We generated single-vector effector protein plus crRNA expressing constructs utilizing nuclear localization (NLS) or cytosolic/nuclear-export signals (NES) to compare knock-down efficacies with uniform delivery, promoter and polyadenylation. Cells were co-transfected with plasmids encoding a Cas13 enzyme together with a crRNA, and with a destabilized GFP plasmid. GFP intensity was recorded by fluorescence activated cell sorting 48 hours after transfection. The percentage of mean fluorescence intensity reduction of cells transfected with one of three different GFP-targeting guide RNAs sequences (G1, G2, G3 ) was determined relative to a non-targeting guide RNA sequence for the same Cas13-fusion protein as a mean of three replicate experiments. We also assessed targeted knock-down with different guide RNA lengths, while maintaining a fixed 5′ or 3′ anchor. RfxCas13d-NLS expressing HEK293 cells were co-transfected with plasmids delivering the crRNA only and a GFP expression plasmid. We cloned the effector proteins (PguCas13b: Addgene 103861, PspCas13b: Addgene 103862, RfxCas13d: Addgene 109049 ) and their direct repeat sequences (PguCas13b: Addgene 103853, PspCas13b: Addgene 103854, RfxCas13d: Addgene 109053 ) as described above. We co-transfected the pLentiRNACRISPR constructs together with a GFP expression plasmid in a 2:1 molar ratio. The guide RNA length comparison was done using previously published RfxCas13d constructs (Addgene 109049 and 109053 ), except that we removed the GFP cassette from the RfxCas13d plasmid. The modified RfxCas13d construct and guide plasmids were co-transfected together with a GFP expression plasmid in a 2:2:1 molar ratio. For the DR modification experiment (FIG. 5c ) we transfected RfxCas13d expressing cells, starting doxycycline-induction (1 μg/ml) at the time of cell plating. The guide plasmid and GFP expression plasmid were co-transfected at a 1:1 molar ratio.

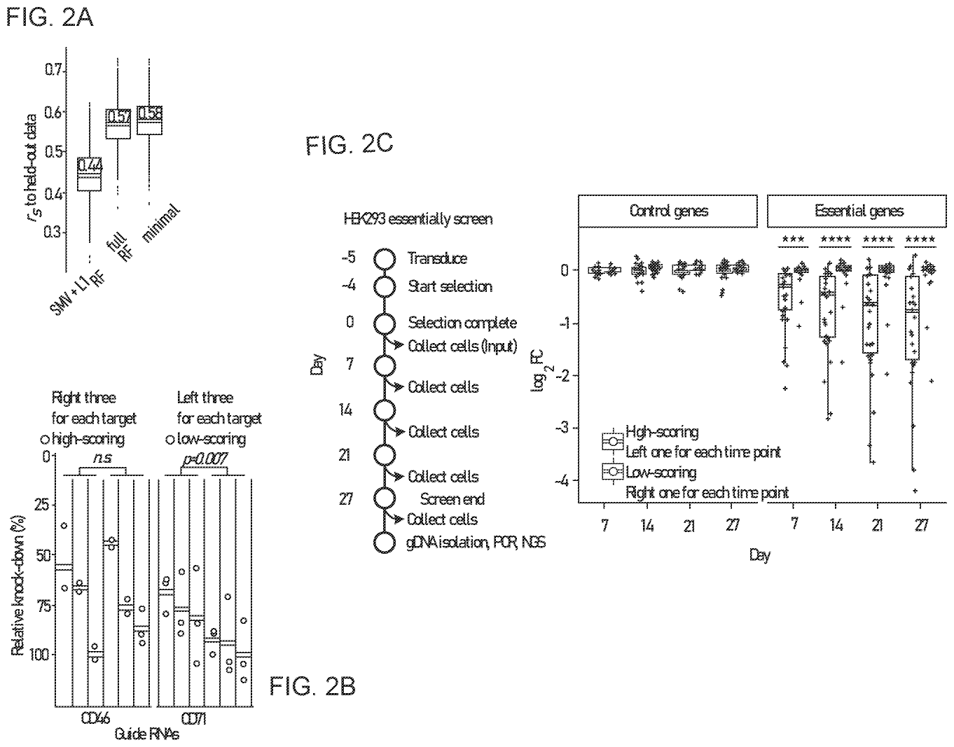

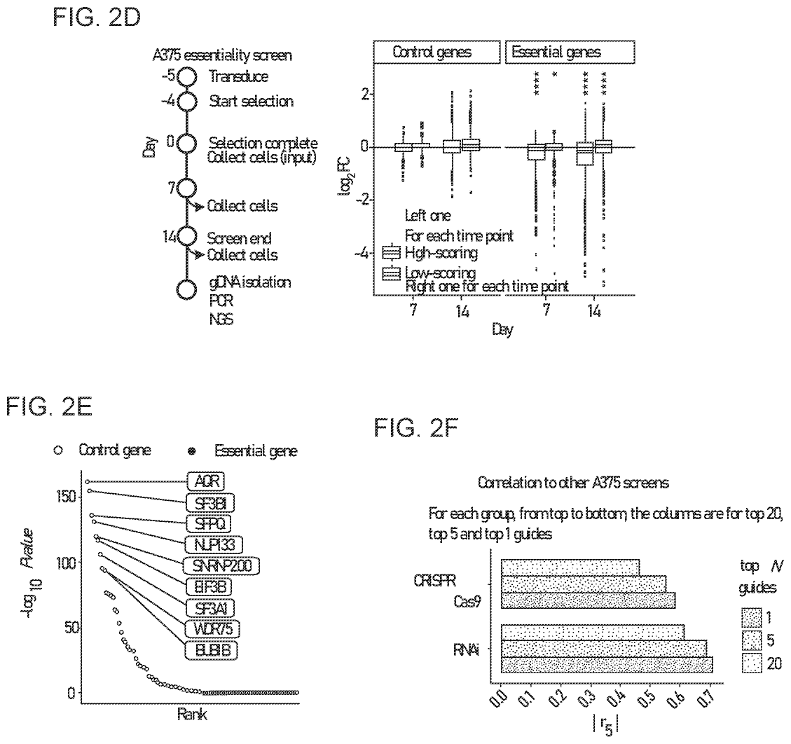

For the model validation flow cytometry (FIG. 2b ) we transfected RfxCas13d-expressing cells with a guide RNA expressing plasmid. 48 hours post transfection, the cells were stained for the respective cell surface protein for 30 min at 4° C. and measured by FACS. (BioLegend: CD46 #352405 clone TRA-2-10, CD71 (TFRC) #334105 clone CYIG4 ).

For the screen result validation (FIG. 1e ) and seed validation experiments (FIG. 1h ) we co-transfected RfxCas13d-expressing cells with a guide RNA expressing plasmid and GFP plasmid at a 1:1 molar ratio. At 48 hours post-transfection, the cells were analyzed by flow cytometry.

To assess the upstream U-context (Example 6 ), we transfected upstream-U context modified EFS-EGFPd2PEST-MCS plasmid together with either a crRNA plasmid into RfxCas13d-expressing in a 2:1 molar ratio. Each GFP-upstreamU-context plasmid was co-transfected with both a targeting or a non-targeting guide RNA used for calculating the knock-down, as a change in 3′UTR uridine content could attract RNA-binding proteins that may affect RNA stability independent of Cas13. We selected the zero-uridine oligonucleotide from a set of 10000 in silico randomized 52mers with {A₂₄,C₁₄,G₁₄} with minimal predicted RNA-secondary structure as determined by RNAfold⁷ with default setting.

For flow cytometry analysis, cells were gated by forward and side scatter and signal intensity to remove potential multiplets. If present, cells were additionally gated with a live-dead staining (LIVE/DEAD Fixable Violet Dead Cell Stain Kit, Thermo Fisher L34963 ). For each sample we analyzed at least 5000 cells. If cell numbers varied, we randomly sampled all samples to the same number of cells before calculating the mean fluorescence intensity (MFI). For GFP co-transfection experiments, we only considered the percentage of transfected cells with the highest GFP expression determined by comparing the non-targeting control to wild-type control cells. For the upstream U-context co-transfection experiments, we considered the whole cell populations.

For knock-down experiments of endogenous genes (FIG. 2b ), we determined the percentage of transfected cells with lower target gene signal than the non-targeting control in the condition with the highest observed knock-down. For all conditions, we analyzed the same bottom percentage of cells. For the selected cells, we compared the MFI of targeting guides relative to non-targeting guides to determine the percent knock-down. To directly compare relative rank of individual guides as done in FIG. 2b, we normalized the effect size by setting the most effective guide to 100%. For the seed validation (FIG. 10, we determined the percentage of transfected (GFP-positive) cells with GFP signal higher than Lipofectamine vehicle treated control cells. The percentage of transfected cells was normalized to percentage of GFP-positive cells in the non-targeting guide control.

D. Screen Library Design and Pooled Oligo Cloning

To design the RfxCas13d guide RNA library for GFP, we selected the 714 bp coding sequence (without start codon) to be targeted. In silico, we generated all perfectly matching 27mer guide RNAs with minimal constraints (T-homopolymer <4, V-homopolymer <5, 0.1<GC-content <0.9 ) and selected 400 by random sampling. From these, we sampled 100 guide RNAs and introduced one random nucleotide conversion at each position (n=2700, SM set). From these 100, we randomly sampled 17 guide RNAs and introduced 26 or 25 consecutive double (n=442, CD set) and triple (n=425, CT set) mismatches, respectively. We sampled an additional 13 guide RNAs from the SM set (in total, 30 guide RNAs) and introduced 100 random double mismatches at any position for each guide RNA if not present already in the set of 17 consecutive double mismatches (n=3000, RD set). In total, we designed 6,967 GFP targeting guides and added 533 non-targeting guides (NT set) of the same length from randomly generated sequences that did not align to the human genome (hg19 ) with less than 3 mismatches.

For CD46, CD55 and CD71 library design, we selected the transcript isoform with highest isoform expression in HEK-TE samples (determined by Cancer Cell Line Encyclopedia CCLE; GENCODE v19 ) and longest 3′UTR isoform (CD46: ENST00000367042.1, CD55: ENST00000367064.3, CD71: ENST00000360110.4 ). As described above, we generated all perfectly matching 23mers, and selected ˜2000 evenly spaced guide RNAs per target. In addition to PM, SM, RD and NT sets as described above, we included for each target a set of guide length variants (n=450, LV set), guide RNAs targeting intronic sequences near splice-donor and splice-acceptor sites across all 39 annotated introns (n=2122, I set) and an additional negative control set of reverse complementary perfect match sequences (n=300, RC set). Further details are gathered but not shown.

For both targeted essentiality screens, we used the DEMETER2 v5³⁷ data set from the Cancer Dependency Map portal (DepMap) to determined essential and control genes. Specifically, we selected essential genes with low log₂ fold-change (FC) enrichments across all cell lines and in the respective assay cell line (s). For our HEK293FT cells, we considered data for HEK-TE cells. Furthermore, we selected genes with one transcript isoform constituting more than 75% of the gene expression with expression level less than ˜150 transcripts per million (TPM). We predicted guide RNA efficiencies using the minimal RF_{GFP} model and removed all guides with matches or partial matches elsewhere in the transcriptome. We allowed up to 3 mismatches when looking for potential off-targets. From the set of remaining perfect match guide RNA predictions, we manually selected three high-scoring and three low-scoring guides for the HEK293FT cell line screen to ensure that each guide fell into non-overlapping regions of the target transcripts. For the A375 cell line targets, we selected the top 20 high-scoring guide RNAs. For the set of 20 low-scoring guides, we chose among the bottom 60 to reduce the overlap of guide RNAs that fall into the same region. In this way, we assayed 20 genes in HEK293FT cells targeting 10 essential and 10 control genes with three low-scoring and three high-scoring guides, as well as three non-targeting guides (n=123 ). For the A375 screen, we targeted 100 genes (35 essential and 65 control genes) with 40 guides each (20 high- and 20 low-scoring) and included 680 non-targeting sequences (n=4680 ).

The guides for the HEK293FT essentiality screen were ordered from IDT, array cloned, confirmed by Sanger sequencing, and subsequently pooled using equal amounts. All other crRNA sequences were synthesized as single-stranded oligonucleotides (Twist Biosciences), PCR amplified using NEBNext High-Fidelity 2×PCR Master Mix (M0541S) (data not shown), and Gibson cloned into pLentiRfxGuide-Puro. Complete library representation with minimal bias (90^{th} percentile/10^{th} percentile crRNA read ratio: 1.68-2.17 ) were verified by next generation sequencing (Illumina MiSeq).

E. Pooled Lentiviral Production and Screening

Lentivirus was produced via transfection of library plasmid with appropriate packaging plasmids (psPAX2: Addgene 12260; pMD2.G: Addgene 12259 ) using polyethylenimine (PEI) reagent in HEK293FT. At 3 days post-transfection, viral supernatant was collected and passed through a 0.45 um filter and stored at −80° C. until use.

Doxycycline-inducible RfxCas13d-NLS human HEK293FT, double-transgenic HEK293FT-GFP or A375 cells were transduced with the respective library pooled lentiviruses in separate infection replicates ensuring at least 1000× guide representation in the selected cell pool per infection replicate using a standard spinfection protocol. We generated either 2 or 3 independent replicate experiments. After 24 hours, RfxCas13d expression was induced by addition of 1 μg/ml doxycycline (Sigma D9891 ) and cells were selected with 1 ug/mL puromycin (ThermoFisher A1113803 ), resulting in ˜30% cell survival. Puromycin-selection was complete ˜48 post puromycin-addition. Assuming independent infection events (Poisson), we determined that ˜83% of surviving cells received a single sgRNA construct. Cells were passaged every two days maintaining at least the initial cell representation and supplemented with fresh doxycycline.

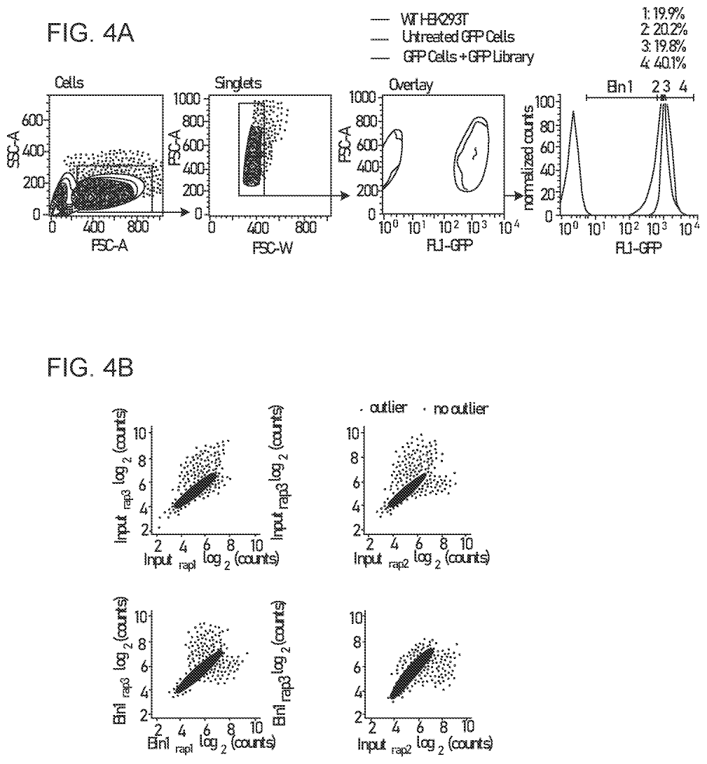

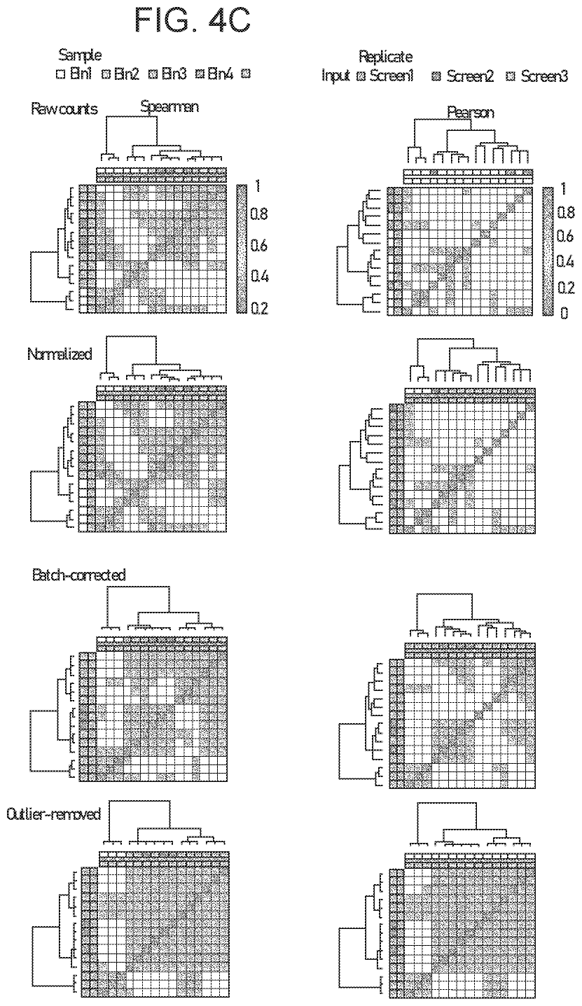

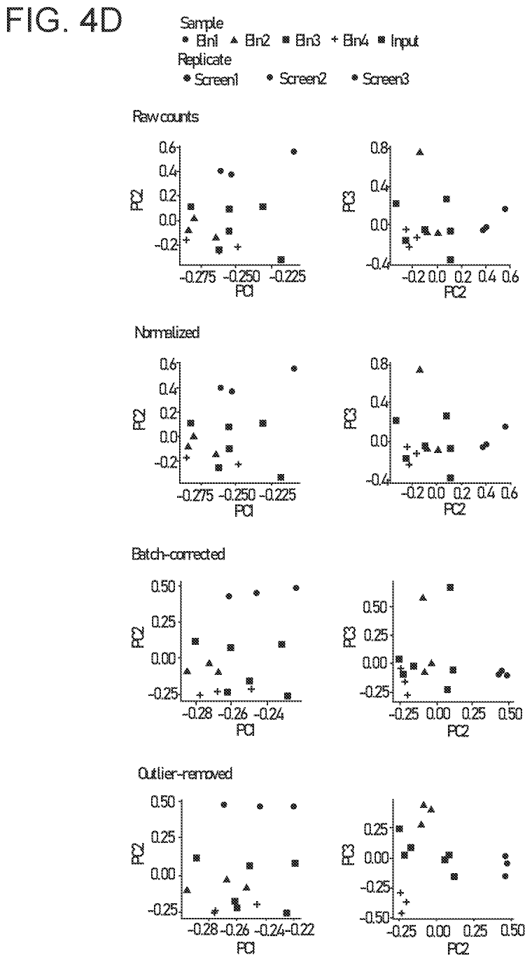

The tiling screens were terminated after 5 to 10 days. For all targets we noted maximal knock-down after 2-4 days (data not shown). For cell surface proteins, cells were stained in batches of 1×10⁷ cells for 30 min at 4° C. (BioLegend: CD46 clone TRA-2-10 #352405-30 per 1×10⁶ cells; CD55 clone JS11 #311311-1.5 μg per 1×10⁶ cells; CD71 clone CYIG4 #334105 -40 per 1×10⁶ cells). We collected unsorted samples for input guide RNA representation of approximately 1000× coverage for each sample and sorted at least another 1000× representation into the assigned bins based on their signal intensities (GFP: lowest 20%, 20%, 20% and remaining highest 40%, FIG. 4a; CD proteins lowest 20% and highest 20%, (data not shown). Cells were PBS-washed and frozen at −80° C. until sequencing library preparation. In each case, the bin containing the lowest 20% represented the strongest target knock-down.

The essentiality screens were started (Day 0 ) upon complete puromycin selection, which was at 5 days after transduction. Cells were passaged every two to three days maintaining at least the initial cell representation and supplemented with fresh doxycycline. At Day 0 (=Input) and every 7 days, we collected a >1000× representation from each sample. The HEK293FT cell screen was conducted in triplicate and cultured for 4 weeks. The A375 cell screen was conducted in duplicate and cultured for 2 weeks.

F. Screen Readout and Read Analysis

For each sample, genomic DNA was isolated from sorted cell pellets using the GeneJET Genomic DNA Purification Kit (ThermoFisher K0722 ) using 2×10⁶ cells or less per column. The crRNA readout was performed using two rounds of PCR². For the first PCR step, a region containing the crRNA cassette in the lentiviral genomic integrant was amplified from extracted genomic DNA using the PCR1 primers (available but not shown).

For each sample, we performed PCR1 reactions as follows: 20 μl volume with 2 ug of gDNA in each reaction limited by the amount of extracted gDNA (total gDNA ranged from 8 μg to 50 ug per sample with an estimated representation of 10⁶ diploid cells per ˜6.6 ug gDNA. PCR1: 4 μl 5× Q5 buffer, 0.02U/0 Q5 enzyme (M0491L), 0.5 uM forward and reverse primers and 100 ng gDNA/μ1. PCR conditions: 98° C./30s, 24×[98° C./10s, 55° C./30s, 72° C./45s], 72° C./5 min).

We pooled the unpurified PCR1 products and used the mixture for a single second PCR reaction per sample. This second PCR adds on Illumina sequencing adaptors, barcodes and stagger sequences to prevent monotemplate sequencing issues. Complete sequences of the 5 forward and 3 reverse Illumina PCR2 readout primers used are not shown. (PCR2: 50 μl 2× Q5 master mix (NEB #M0492S), 10 μl PCR1-product, 0.5 uM forward and reverse PCR2-primers in 1000. PCR conditions: 98° C./30s, 17×[98° C./10s, 63° C./30s, 72° C./45s], 72° C./5 min).

Amplicons from the second PCR were pooled by screen experiment (e.g. all GFP-screen samples) in equimolar ratios (by gel-based band densitometry quantification) and then purified using a QiaQuick PCR Purification kit (Qiagen 28104 ). Purified products were loaded onto a 2% E-gel and gel extracted using a QiaQuick Gel Extraction kit (Qiagen 28704 ). The molarity of the gel-extracted PCR product was quantified using KAPA library quant (KK4824 ) and sequenced on an Illumina NextSeq 500—II MidOutput 1×150 v2.5.

Reads were demultiplexed based on Illumina i7 barcodes present in PCR2 reverse primers using bcl2fastq and by their custom in-read i5 barcode using a custom python script. Reads were trimmed to the expected guide RNA length by searching for known anchor sequences relative to the guide sequence using a custom python script. For the tiling screens, pre-processed reads were either aligned to the designed crRNA reference using bowtie³ (v.1.1.2 ) with parameters -v 0 -m 1 or collapsed (FASTX-Toolkit) to count perfect duplicates followed by string-match intersection with the reference to retain only perfectly matching and unique alignments. Pre-processed guide RNA sequences from the essentiality screens were aligned allowing for up to 1 mismatch (-v 1 -m 1 ). Alignment statistics are available but not shown. The raw guide RNA counts (data not shown) were normalized separated by screen dataset using a median of ratios method like in DESeq2⁴ and underwent batch-correction using combat implemented in the SVA R package⁵. Non-reproducible technical outliers were removed by applying pair-wise linear regression for each sample after normalization and batch-correction, collecting the residuals and taking the median value for each guide RNA across all sample-centric comparisons. We removed all crRNA counts within the top X % residuals across all samples (GFP: 2%, CD proteins: 0.5%, Essentiality screen: no outlier removal). For the GFP screen, we only remove outliers on a per-sample basis as needed (but not the entire guide RNA). For CD46, CD55 and CD71 screens, since the number of outliers was small, we decided to remove the entire guide RNA from the analysis. The table below indicates all filtering applied:

TABLE 2a

| | | <N reads in | | | | | |
|---|---|---|---|---|---|---|---|
| | not | input | 0 reads in any | masked | | | |
| Screen | detected* | samples* | sample* | outlier | filtered | total | remaining |
| GFP | 0 | not applied | not applied | 4** | 4 | 7500 | 7496 |
| CD46 | 19 | 427 (<50) | 22 | 77 | 545 | 5605 | 5060 |
| CD55 | 23 | 88 (<50) | 0 | 79 | 190 | 5356 | 5166 |
| CD71 | 3 | 48 (<50) | 0 | 75 | 123 | 5999 | 5876 |
| HEK293 | 0 | 0 (<50) | 0 | 0 | 0 | 123 | 123 |
| A375 | 2 | 10 (<100) | 0 | 0 | 12 | 4680 | 4668 |

*Removed before normalization
**filtered for Bin1 guide RNAs

Processed crRNA counts are available but not shown. Guide RNA enrichments were calculated building the count ratios between a bin or timepoint and the corresponding input sample and loge-transformation (log₂FC). Consistency between replicates was estimated using robust rank aggregation (RRA)⁶. Delta log₂FC for mismatching guides was calculated by subtracting the log₂FC of the perfectly matching reference guide. For the tiling screens, all plots and analyses were performed using the mean guide RNA enrichments of bin 1 (=bottom 20%) across replicates, unless indicated otherwise. Similarly, we used the mean guide RNA enrichments relative to Day 0 across replicates for the essentiality screen. Guide RNA enrichment scores (log₂FC) are not shown here. In all combined analyses across all four tiling screens, we scaled the observed log₂FC separately to improve comparability. For the generation of a the combined on-target model, we normalized the 2918 selected CDS-targeting guides RNA across the four tiling screens to the same scale prior to training and testing the model. To do so, for each dataset D, we computed the upper and lower quartiles of the guide log₂FC (UQ_{D} and LQ_{D}, respectively) as well as the corresponding quartiles for the log₂FC among all datasets pooled together (UQ_{P} and LQ_{P}). We then updated each fold change x as follows: x\hat{ }=[(x−LQ_{D} )/(UQ_{D}−LQ_{D})*(UQ_{P}−LQ_{P})+LQ_{P}]. By centering on quartiles, this procedure normalized the fold-change distributions in a way that was less susceptible to the influence of outliers of a single screen.

G. Predicting RNA secondary structures and RNA-RNA hybridization energies crRNA secondary structure and minimum free energies (MFEs) was derived using RNAfold [--gquad] on the full-length crRNA (DR+guide) sequence⁷. For building the combined on-target model and for testing the RF_{GFP} model on the combined data set, we assumed 23mer guide RNAs for all guides in the GFP tiling screen to prevent length dependent differences in the crRNA MFE. Target RNA unpaired probability (accessibility) was calculated using RNAplfold [-L 40 -W 80 -u 50] as described before⁸. We performed a grid-search calculating the RNA accessibility for each target nucleotide in a window of minus 20 bases downstream of the target site to plus 20 bases upstream of the target site assessing the unpaired probability of each nucleotide over 1 to 50 bases for all perfectly matching guides. Then, we calculated the Pearson correlation coefficient between the log₁₀-transformed unpaired probabilities and the observed guide RNA log₂FC for each point and window relative to the guide RNA. RNA-RNA-hybridization between the guide RNA and its target site was calculated using RNAhybrid [-s -c] ⁹. For the hybridization calculation, we did not include the direct repeat of the crRNA. We calculated the RNA-hybridization minimum free energy for each guide RNA nucleotide position p over the distance d to the position p+d with its cognate target sequence. All measures were either directly correlated with the observed crRNA log₂FC or using partial correlation to account for the crRNA folding MFE. In each case, we computed the Pearson correlation.

H. Assessing Guide RNA Nucleotide Composition

Guide RNA composition was derived by calculating the nucleotide probability within the respective guide RNA sequence length. To assess the presence of sequence constraints similar to a previously described anti-tag¹⁰ or 5′ and 3′ Protospacer Flanking Sequences (PFS), we ranked all perfectly matching guide RNAs by their log₂FC enrichment within each screen separately. We selected the top and bottom 20% enriched/depleted guide RNAs and calculated the positional nucleotide probability for the four nucleotides upstream and downstream relative to the guide RNA match. To assess nucleotide preferences at any guide RNA match position in addition to upstream and downstream nucleotides, we selected the top 20% of the log₂FC-ranked perfectly matching guides as described above and calculated nucleotide preferences as described before¹¹. In brief, we calculated the probability of each nucleotide at each position for the top guide RNAs and all guide RNAs. The effect size is the difference of nucleotide probability by subtracting the values from all guides from the top guides (delta log₂FC). p-values were calculated from the binomial distribution with a baseline probability estimated from the full-length GFP mRNA target sequence for all perfectly matching crRNAs. p-values were adjusted using a Bonferroni multiple testing hypothesis correction.

I. Assessing Target RNA Context

To assess the target RNA context, we calculated the nucleotide probability at each position (p) over a window (w) of 1 to 50 nucleotides centered around the position of interest (e.g. p=−18 with w=11 summarizes the nucleotide content in a window from −23 to −13 with +1 being the first base of the crRNA). We evaluated p for all positions within 75 nucleotides upstream and downstream of the guide RNA. The nucleotide context of each point was then correlated with the observed log₂FC crRNA enrichments for all perfect match crRNAs, either directly or using partial correlation accounting for crRNA folding MFE. In each case we used Pearson correlation.

The RNA context around single nucleotide mismatches was assessed accordingly with a slight modification. Here, the nucleotide context was assessed relative to mismatch position summarizing the nucleotide probability in a window of 1 to 15 nucleotides to either side (e.g. p=18 with w=5 summarizes the nucleotide content in a window of 11 nucleotides from 23 to 13 ). The nucleotide context around single nucleotide mismatches influences the mismatch tolerance. We determined Pearson correlation coefficient (rₚ ) between observed log₂FC and delta log₂FC for all single mismatch guide RNAs relative to their cognate perfect matching guide RNAs segregated by all 27 positions. For each mismatch position p relative to the 5′ guide RNA end the nucleotide density was calculated as a fraction in a window extended by d (1-15 nt) on both sides centered on the mismatch position p.

The nucleotide context of each position and each window size was then correlated with the observed delta log₂FC relative to the perfectly matching reference guide RNA, either directly or using partial correlation accounting for crRNA folding MFE. In each case, we used Pearson correlation.

J. On-Target Model Selection

An explanation for all selected features for the RF_{GFP} and RF_{combined} model can be found in Table 6 and Table 7, respectively. The RF_{combined} model feature input values are note shown here. All continuous feature scores were scaled to the [0, 1] interval limited to the 5^{th} and 95^{th} percentile, with a mean set to the 5^{th} percentile. Scaled values exceeding the [0, 1] interval were set to 0 or 1, respectively.

TABLE 2
Scaling parameters used to normalize data to the [0,
1] interval for the Random Forest Models

| | | 5^{th} | 95^{th} |
|---|---|---|---|
| Model | Feature | percentile | percentile |
| RF_{GFP} | crRNA MFE | −23.4000 | −14.5000 |
| | Local A probability | 0.0000 | 0.4286 |
| | Local C | 0.2273 | 0.5000 |
| | Local G probability | 0.1429 | 0.4286 |
| | Local U probability | 0.0556 | 0.2778 |
| | upstream U probability | 0.0667 | 0.2000 |
| RF_{combined} | crRNA MFE | −20.3000 | −12.8000 |
| | Log₁₀ Unpaired probability | −7.5546 | −1.6244 |
| | hybridization MFE nt 3-15 | −29.4000 | −17.9000 |
| | hybridization MFE nt 15-23 | −21.8000 | −12.3000 |
| | Local Aₘₐₓ probability | 0.0000 | 0.5000 |
| | Local Cₘₐₓ probability | 0.0000 | 0.5000 |
| | Local Gₘₐₓ probability | 0.0000 | 0.6667 |
| | Local Uₘₐₓ probability | 0.0833 | 0.5000 |
| | Local AUₘₐₓ probability | 0.2727 | 0.7272 |
| | Local GCₘₐₓ probability | 0.2222 | 0.7778 |
| | Local Aₘᵢₙ probability | 0.0000 | 0.5714 |
| | Local Cₘᵢₙ probability | 0.0000 | 0.5556 |
| | Local Gₘᵢₙ probability | 0.0000 | 0.4444 |
| | Local Uₘᵢₙ probability | 0.0000 | 0.5000 |
| | Local AUₘᵢₙ probability | 0.2222 | 0.7778 |
| | Guide A nt probability | 0.0870 | 0.4348 |
| | Guide C nt probability | 0.0870 | 0.3913 |
| | Guide G nt probability | 0.0870 | 0.4348 |
| | Guide AA di-nt probability | 0.0000 | 0.1818 |
| | Guide AC di-nt probability | 0.0000 | 0.1364 |
| | Guide AG di-nt probability | 0.0000 | 0.1818 |
| | Guide AU di-nt probability | 0.0000 | 0.1364 |
| | Guide CA di-nt probability | 0.0000 | 0.1818 |
| | Guide CC di-nt probability | 0.0000 | 0.1364 |
| | Guide CG di-nt probability | 0.0000 | 0.1364 |
| | Guide CU di-nt probability | 0.0000 | 0.1364 |
| | Guide GA di-nt probability | 0.0000 | 0.1364 |
| | Guide GC di-nt probability | 0.0000 | 0.1818 |
| | Guide GG di-nt probability | 0.0000 | 0.1818 |
| | Guide GU di-nt probability | 0.0000 | 0.1364 |
| | Guide UA di-nt probability | 0.0000 | 0.1364 |
| | Guide UC di-nt probability | 0.0000 | 0.1818 |
| | Guide UG di-nt probability | 0.0000 | 0.1818 |

To evaluate and compare model performances, we randomly sampled 1,000 bootstrap datasets from the data of perfect match guide RNA log₂FC response values and selected features. We used 399 data points for the initial RF_{GFP} model and 2918 data points for all CDS-annotating perfect match guides across the four tiling screens. For the RF_{combined} model we normalized the observed log₂FC values data prior to training and testing as described earlier. Normalized response values showed better generalizability compared to unnormalized or scaled log₂FC. For each bootstrap sample, 70% of the data was used for training and the remaining 30% of the data was held out for testing, ensuring a 70/30 split for each screen dataset when testing the RF_{combined} model. Linear dependencies between features were identified using the function findLinearCombos from the R package caret and removed. The model performance was evaluated by calculating the Spearman correlation coefficient rₛ and Pearson r² to the held-out data. We compared a variety of different methods⁸ (Table 3 ).

TABLE 3

| # | Name | Function | Parameter | R package |
|---|---|---|---|---|
| 1 | all subsets regression, | regsubsets | nvmax = 15, nbest = 1, | leaps |
| | maximizing the Bayesian | | method = “forward”, | |
| | information criterion (BIC) | | really.big = T | |
| 2 | stepwise regression, | stepAIC | — | MASS |
| | maximizing the BIC | | | |
| 3 | stepwise regression, | stepAIC | — | MASS |
| | maximizing the Akaike | | | |
| | information criterion (AIC) | | | |
| 4 | Lasso regression | cv.glmnet | family = “gaussian”, | glmnet |
| | | | nfolds = 10, | |
| | | | alpha = 1 | |
| 5 | multivariate adaptive | Earth | degree = 1, trace = 0, | earth |
| | regression splines (MARS) | | nk = 500 | |
| 6 | Random Forest | randomForest | — | randomForest |
| 7 | principal component | Pcr | ncomp = 5 (during | pls |
| | regression (PCR) | | prediction) | |
| 8 | Partial Least Squares (PLSR) | Plsr | ncomp = 5 (during | pls |
| | | | prediction) | |
| 9 | Support Vector Machine w/ | Tune | method = svm, ranges = | e1071 |
| | L1 loss function (SMV + L1) | | list(epsilon = seq(0, 1, 0.025), | |
| | | | cost = 2\hat{ }(2:8)), kernel = | |
| | | | “radial” | |

For both models, we tested a variety of feature combinations including crRNA folding energies, RNA-RNA hybridization energies, target site accessibility, overall and positional (di-) nucleotide probabilities, and one-hot encoding for single and di-nucleotide of the guide target-sites and their upstream and downstream flanking four nucleotides. Together, these represented 644 features for the combined on-target model. A full set of features for the combined on-target model can be found below.

### Feature List

| | | | | | | | | | | | | | | |
|---|---|---|---|---|---|---|---|---|---|---|---|---|---|---|
| meanCS | GC | 3 | T_17 | C_4 | AT_−2 | CC_22 | CT_19 | GC_16 | GT_13 | TC_10 | TT_7 | AC_3 | AT_+1 | CC_+4 |
| pVal | GG | 2 | A_16 | G_4 | CA_−2 | CG_22 | GA_19 | GG_16 | TA_13 | TG_10 | AA_6 | AG_3 | CA_+1 | CG_+4 |
| log10pVal | GT | 1 | C_16 | T_4 | CC_−2 | CT_22 | GC_19 | GT_16 | TC_13 | TT_10 | AC_6 | AT_3 | CC_+1 | CT_+4 |
| ScaledCS.all | TA | 1 | G_16 | A_3 | CG_−2 | GA_22 | GG_19 | TA_16 | TG_13 | AA_9 | AG_6 | CA_3 | CG_+1 | GA_+4 |
| ScaledCS | TC | 2 | T_16 | C_3 | CT_−2 | GC_22 | GT_19 | TC_16 | TT_13 | AC_9 | AT_6 | CC_3 | CT_+1 | GC_+4 |
| normCS | TG | 3 | A_15 | G_3 | GA_−2 | GG_22 | TA_19 | TG_16 | AA_12 | AG_9 | CA_6 | CG_3 | GA_+1 | GG_+4 |
| MFE | TT | 4 | C_15 | T_3 | GC_−2 | GT_22 | TC_19 | TT_16 | AC_12 | AT_9 | CC_6 | CT_3 | GC_+1 | GT_+4 |
| DR | pAA | A_−4 | G_15 | A_2 | GG_−2 | TA_22 | TG_19 | AA_15 | AG_12 | CA_9 | CG_6 | GA_3 | GG_+1 | TA_+4 |
| Gquad | pAC | C_−4 | T_15 | C_2 | GT_−2 | TC_22 | TT_19 | AC_15 | AT_12 | CC_9 | CT_6 | GC_3 | GT_+1 | TC_+4 |
| Log10_Unpaired | pAG | G_−4 | A_14 | G_2 | TA_−2 | TG_22 | AA_18 | AG_15 | CA_12 | CG_9 | GA_6 | GG_3 | TA_+1 | TG_+4 |
| hybMFE_3.12 | pAT | T_−4 | C_14 | T_2 | TC_−2 | TT_22 | AC_18 | AT_15 | CC_12 | CT_9 | GC_6 | GT_3 | TC_+1 | TT_+4 |
| hybMFE_15.9 | pCA | A_−3 | G_14 | A_1 | TG_−2 | AA_21 | AG_18 | CA_15 | CG_12 | GA_9 | GG_6 | TA_3 | TG_+1 | |
| NTdens_max_A | pCC | C_−3 | T_14 | C_1 | TT_−2 | AC_21 | AT_18 | CC_15 | CT_12 | GC_9 | GT_6 | TC_3 | TT_+1 | |
| NTdens_max_C | pCG | G_−3 | A_13 | G_1 | AA_−1 | AG_21 | CA_18 | CG_15 | GA_12 | GG_9 | TA_6 | TG_3 | AA_+2 | |
| NTdens_max_G | pCT | T_−3 | C_13 | T_1 | AC_−1 | AT_21 | CC_18 | CT_15 | GC_12 | GT_9 | TC_6 | TT_3 | AC_+2 | |
| NTdens_max_T | pGA | A_−2 | G_13 | A_+1 | AG_−1 | CA_21 | CG_18 | GA_15 | GG_12 | TA_9 | TG_6 | AA_2 | AG_+2 | |
| NTdens_max_AT | pGC | C_−2 | T_13 | C_+1 | AT_−1 | CC_21 | CT_18 | GC_15 | GT_12 | TC_9 | TT_6 | AC_2 | AT_+2 | |
| NTdens_max_GC | pGG | G_−2 | A_12 | G_+1 | CA_−1 | CG_21 | GA_18 | GG_15 | TA_12 | TG_9 | AA_5 | AG_2 | CA_+2 | |
| NTdens_min_A | pGT | T_−2 | C_12 | T_+1 | CC_−1 | CT_21 | GC_18 | GT_15 | TC_12 | TT_9 | AC_5 | AT_2 | CC_+2 | |
| NTdens_min_C | pTA | A_−1 | G_12 | A_+2 | CG_−1 | GA_21 | GG_18 | TA_15 | TG_12 | AA_8 | AG_5 | CA_2 | CG_+2 | |
| NTdens_min_G | pTC | C_−1 | T_12 | C_+2 | CT_−1 | GC_21 | GT_18 | TC_15 | TT_12 | AC_8 | AT_5 | CC_2 | CT_+2 | |
| NTdens_min_T | pTG | G_−1 | A_11 | G_+2 | GA_−1 | GG_21 | TA_18 | TG_15 | AA_11 | AG_8 | CA_5 | CG_2 | GA_+2 | |
| NTdens_min_AT | pTT | T_−1 | C_11 | T_+2 | GC_−1 | GT_21 | TC_18 | TT_15 | AC_11 | AT_8 | CC_5 | CT_2 | GC_+2 | |
| NTdens_min_GC | Guide | A_23 | G_11 | A_+3 | GG_−1 | TA_21 | TG_18 | AA_14 | AG_11 | CA_8 | CG_5 | GA_2 | GG_+2 | |
| | Seq | | | | | | | | | | | | | |
| GFP_local_max_A | RetrievedPos | C_23 | T_11 | C_+3 | GT_−1 | TC_21 | TT_18 | AC_14 | AT_11 | CC_8 | CT_5 | GC_2 | GT_+2 | |
| GFP_local_max_C | Target | G_23 | A_10 | G_+3 | TA_−1 | TG_21 | AA_17 | AG_14 | CA_11 | CG_8 | GA_5 | GG_2 | TA_+2 | |
| | Seq | | | | | | | | | | | | | |
| GFP_local_max_G | −4 | T_23 | C_10 | T_+3 | TC_−1 | TT_21 | AC_17 | AT_14 | CC_11 | CT_8 | GC_5 | GT_2 | TC_+2 | |
| GFP_local_max_T | −3 | A_22 | G_10 | A_+4 | TG_−1 | AA_20 | AG_17 | CA_14 | CG_11 | GA_8 | GG_5 | TA_2 | TG_+2 | |
| GFP_local_max_uU | −2 | C_22 | T_10 | C_+4 | TT_−1 | AC_20 | AT_17 | CC_14 | CT_11 | GC_8 | GT_5 | TC_2 | TT_+2 | |
| A | −1 | G_22 | A_9 | G_+4 | AA_23 | AG_20 | CA_17 | CG_14 | GA_11 | GG_8 | TA_5 | TG_2 | AA_+3 | |
| C | 23 | T_22 | C_9 | T_+4 | AC_23 | AT_20 | CC_17 | CT_14 | GC_11 | GT_8 | TC_5 | TT_2 | AC_+3 | |
| G | 22 | A_21 | G_9 | AA_−3 | AG_23 | CA_20 | CG_17 | GA_14 | GG_11 | TA_8 | TG_5 | AA_1 | AG_+3 | |
| T | 21 | C_21 | T_9 | AC_−3 | AT_23 | CC_20 | CT_17 | GC_14 | GT_11 | TC_8 | TT_5 | AC_1 | AT_+3 | |
| pA | 20 | G_21 | A_8 | AG_−3 | CA_23 | CG_20 | GA_17 | GG_14 | TA_11 | TG_8 | AA_4 | AG_1 | CA_+3 | |
| pC | 19 | T_21 | C_8 | AT_−3 | CC_23 | CT_20 | GC_17 | GT_14 | TC_11 | TT_8 | AC_4 | AT_1 | CC_+3 | |
| pG | 18 | A_20 | G_8 | CA_−3 | CG_23 | GA_20 | GG_17 | TA_14 | TG_11 | AA_7 | AG_4 | CA_1 | CG_+3 | |
| pT | 17 | C_20 | T_8 | CC_−3 | CT_23 | GC_20 | GT_17 | TC_14 | TT_11 | AC_7 | AT_4 | CC_1 | CT_+3 | |
| G\|C | 16 | G_20 | A_7 | CG_−3 | GA_23 | GG_20 | TA_17 | TG_14 | AA_10 | AG_7 | CA_4 | CG_1 | GA_+3 | |
| A\|T | 15 | T_20 | C_7 | CT_−3 | GC_23 | GT_20 | TC_17 | TT_14 | AC_10 | AT_7 | CC_4 | CT_1 | GC_+3 | |
| pG\|pC | 14 | A_19 | G_7 | GA_−3 | GG_23 | TA_20 | TG_17 | AA_13 | AG_10 | CA_7 | CG_4 | GA_1 | GG_+3 | |
| pA\|pT | 13 | C_19 | T_7 | GC_−3 | GT_23 | TC_20 | TT_17 | AC_13 | AT_10 | CC_7 | CT_4 | GC_1 | GT_+3 | |
| AA | 12 | G_19 | A_6 | GG_−3 | TA_23 | TG_20 | AA_16 | AG_13 | CA_10 | CG_7 | GA_4 | GG_1 | TA_+3 | |
| AC | 11 | T_19 | C_6 | GT_−3 | TC_23 | TT_20 | AC_16 | AT_13 | CC_10 | CT_7 | GC_4 | GT_1 | TC_+3 | |
| AG | 10 | A_18 | G_6 | TA_−3 | TG_23 | AA_19 | AG_16 | CA_13 | CG_10 | GA_7 | GG_4 | TA_1 | TG_+3 | |
| AT | 9 | C_18 | T_6 | TC_−3 | TT_23 | AC_19 | AT_16 | CC_13 | CT_−10 | GC_7 | GT_4 | TC_1 | TT_+3 | |
| CA | 8 | G_18 | A_5 | TG_−3 | AA_22 | AG_19 | CA_16 | CG_13 | GA_10 | GG_7 | TA_4 | TG_1 | AA_+4 | |
| CC | 7 | T_18 | C_5 | TT_−3 | AC_22 | AT_19 | CC_16 | CT_13 | GC_10 | GT_7 | TC_4 | TT_1 | AC_+4 | |
| CG | 6 | A_17 | G_5 | AA_−2 | AG_22 | CA_19 | CG_16 | GA_13 | GG_10 | TA_7 | TG_4 | AA_+1 | AG_+4 | |
| CT | 5 | C_17 | T_5 | AC_−2 | AT_22 | CC_19 | CT_16 | GC_13 | GT_10 | TC_7 | TT_4 | AC_+1 | AT_+4 | |
| GA | 4 | G_17 | A_4 | AG_−2 | CA_22 | CG_19 | GA_16 | GG_13 | TA_10 | TG_7 | AA_3 | AG_+1 | CA_+4 | |

For the initial on target model based on the GFP screen data, we evaluate a set of 15 defined features (Table 6 ) along-side with one-hot encoded positional nucleotide information and GC content. These 15 features were defined based on their positive or negative correlation to the observed response value during the data exploration (see also Example 6 ). We iteratively reduced the numbers of features from 15 to 6 for the RF_{GFP} model and monitored the model performance as described above. At each iteration, the Random Forest model performed slightly better than any other learning approach. Reducing the features to fewer than the selected 6 features (RFₘᵢₙᵢₘₐₗ=RF_{GFP} ) reduced the model performance. For the combined on-target model, we did not iteratively reduce the set of 35 selected features. We compared the RF_{GFP} model to an SVM+L1 model similar to one of the first CRISPR-Cas9 on-target model. Specifically, we used one-hot encoding for all 35 nucleotide positions considered (27 guide RNA positions and 8 additional positions with 4 upstream and 4 downstream nucleotides). Considering all positions, the feature space contained 140 single nucleotide features, 544 di-nucleotide features and the GC-content (685 non-all-zero features). Here, we used tuning (see table herein for parameters) to increase model performance for SVM+L1 specifically. Here, but also for the combined model, one-hot encoded features did not lead to high Spearman correlation coefficient rₛ to the held-out data.

For further evaluation of the random forest models we used 10-fold cross-validation by randomly partitioning the data into 10 equally-sized partitions ensuring even contribution from each screen to each partition. We trained the model 10 times on 90% of the data and predicted the held-out 10%. For each data point, we assigned the known guide RNA efficacy quartile based on the log₂FC enrichment and compared it the predicted efficacy quartiles in the held-out data. We also assessed the predicted guide score by calculating the median predicted guide score for the top and bottom ranked crRNAs in the 10% held-out data based on the known log₂FC-rank for all 10 cross-validation folds (top/bottom N=2, 4, 8, 16, 32, 64, 128 or 256 guide RNAs). To compute the null distribution, we calculated the median predicted guides scores of randomly selected guide RNAs across 1000 samplings for each N. For the leave-one-out cross-validation we trained on all data from three tiling screens and performed Spearman rank correlation of the predicted the guide efficiency of the held-out fourth screen to the observed log₂FC enrichments.

To make the guide score more interpretable, we standardized the guide score to a [0, 1] interval preserving the distribution between 5^{th} and 95^{th} percentile. Normalized values exceeding the [0, 1] interval were set to 0 or 1, respectively. The final RF_{GFP} model was trained on all data points for perfect match guides using the six selected features with 1500 regression trees. The model explains 36.9% of the observed variance with a mean of squared residuals of 0.139. The table below shows the feature contribution for the RF_{GFP} model.

TABLE 4

| Feature | % IncMSE | IncNodePurify |
|---|---|---|
| crRNA MFE | 57.989 | 22.617 |
| Local A probability | 30.542 | 7.529 |
| Local C probability | 46.255 | 13.683 |
| Local G probability | 38.557 | 9.256 |
| Local U probability | 29.953 | 6.555 |
| upstream U probability | 31.559 | 8.629 |

Similarly, final RF_{combined} was trained on 2918 data points using 35 selected features. Tuning the number of trees (ntree) and number of splitting variables per node (mtry) led to insignificant insignificant performance improvements compared to default settings. The model (mtry=12, ntree=2000 ) explains 47.16% of the observed variance, a mean of squared residuals of 0.168, and the feature contribution as indicated below ranked by importance:

TABLE 5

| Feature | % IncMSE | IncNodePurity |
|---|---|---|
| Local Gₘₐₓ probability | 86.6814 | 47.9987 |
| crRNA MFE | 72.5480 | 69.6068 |
| Unpaired probability | 72.1078 | 57.5635 |
| Local Cₘᵢₙ probability | 56.9153 | 37.2137 |
| hybridization MFE nt 15-23 | 54.5793 | 94.9857 |
| Guide G nt probability | 47.7324 | 29.2452 |
| Guide CA di-nt probability | 47.6241 | 21.4664 |
| hybridization MFE nt 3-15 | 47.3083 | 46.7462 |
| Guide CG di-nt probability | 44.0711 | 11.7069 |
| Guide AU di-nt probability | 42.1128 | 15.6205 |
| Guide CU di-nt probability | 40.6967 | 16.9172 |
| Guide A nt probability | 39.7297 | 25.0736 |
| Local GCₘₐₓ probability | 39.7236 | 59.0805 |
| Local AUₘᵢₙ probability | 38.8318 | 56.3599 |
| Local Uₘₐₓ probability | 38.7535 | 24.4657 |
| Guide GG di-nt probability | 38.5896 | 22.2621 |
| Local Gₘᵢₙ probability | 36.5244 | 19.6159 |
| Guide UC di-nt probability | 36.3043 | 15.2075 |
| Guide AC di-nt probability | 36.2901 | 14.2361 |
| Guide C nt probability | 36.0901 | 16.8637 |
| Local Uₘᵢₙ probability | 35.6886 | 16.2571 |
| Guide AG di-nt probability | 34.9534 | 14.6260 |
| Local Aₘᵢₙ probability | 34.6632 | 17.4928 |
| Local Aₘₐₓ probability | 33.6557 | 16.1888 |
| Guide AA di-nt probability | 33.2106 | 13.3830 |
| Guide GC di-nt probability | 33.0670 | 13.3209 |
| Guide CC di-nt probability | 32.2834 | 12.9797 |
| Guide UG di-nt probability | 31.9188 | 13.1659 |
| Guide GU di-nt probability | 31.8413 | 12.8934 |
| Guide GA di-nt probability | 31.3837 | 13.1399 |
| Guide UA di-nt probability | 30.6930 | 12.5052 |
| Local Cₘₐₓ probability | 29.8186 | 14.2189 |
| Local AUₘₐₓ probability | 22.4842 | 14.2585 |
| Predicted correctly folded DR | 8.2669 | 4.0679 |
| Predicted G-quadruplex | −3.0274 | 0.0219 |

K. RfxCas13d Guide Scoring

We created a user-friendly R script that readily predicts RfxCas13d on-target guide scores. The only user-provided argument is a single-entry FASTA file input of minimally 30 nt that represents the target sequence, such as a transcript isoform sequence. The software first generates all possible 23mer guide RNAs and collects all required features and predicts guide RNA efficacies. The only filter applied removes guide RNAs with homopolymers of 5 or more Ts and 6 or more Vs (V=A, C, G). Such guide RNAs may trigger early transcript termination for PolIII transcription or cause difficulties during oligo synthesis. The software returns a FASTA file with guide RNA sequences ranked by the predicted standardized guide score. In addition, a csv file is created following providing additional information. Optionally, the script can be used to plot the guide score distribution along the provided target sequence for visualization.

We used this software to predict guide scores for all transcripts (including all biotypes: protein_coding, nonsense_mediated_decay, non_stop_decay, IG_*_gene, TR_*_gene, polymorphic_pseudogene) of protein coding genes annotated in GENCODE v19 (GRCh37 ) (n=94,873 of 95,074 ) and provide the top 10 ranked 5′UTR, coding sequence and 3′UTR annotating guide RNA sequences (data not shown).

L. RfxCas13d Guide Scoring Validation

To validate our that our initial RF_{GFP} model can readily separate between poorly and well-performing crRNAs, we performed several experiments.

First, we chose two genes that encode for cell surface proteins that allow for quantitative assessment of their expression levels by FACS. For each gene we predicted crRNAs for the highest expressed transcript isoform in HEK293FT cells (CD46: ENST00000367042.1, CD71 [TFRC]: ENST00000360110.4 ). For each gene, we selected 3 guides present in the low scoring quartiles (Q1 and Q2 ) and 3 guides in the high scoring quartiles (Q3 and Q4 ). We selected the guides to be non-overlapping and to reside in 3 different regions of the target transcript.

Then, we performed two essentiality screens with a dropout growth phenotype readout in HEK293FT and A375 cells, respectively. We designed two crRNA libraries targeting essential and control genes with a number of predicted low-scoring and high-scoring guide RNAs as described above (see Screen library design and pooled oligo cloning). For the HEK293FT cell screen, we compared the guide depletion of four groups of 30 guides (Essential gene targeted by high-scoring guide or by low-scoring guide, and control genes targeted by high-scoring guide or by low-scoring guide). We expected the greatest depletion for the 30 high-scoring guide RNAs targeting essential genes. Similarly, we compared the relative guide depletion of the same four groups of guide RNAs in the A375 screen, with the expectation that the 20 high-scoring guides per essential gene would be the most depleted.

For gene ranking based on guide depletion, we used robust rank aggregation (RRA)⁶ to assign a p-value based on the consistency of log₂FC-based rank-consistency of the most depleted N guide RNAs per gene (with N {1, 5, 20}) across the two A375 screen replicates. The -log₁₀ transformed p-values were then compared to other growth screens (RNAi and Cas9 ) using Spearman rank correlation. Specifically, we compared the RRA-derived log₁₀ p-value to the log₂FC from an RNAi-based DEMETER2 v5 repository³⁷ and the merged STARS scores from a Cas9-based approach²⁹. For the correlation we only used genes with value present in all scores (Essential genes: n=35; Control genes: n=15 ).

Furthermore, we used the log₂FC guide depletion values to compare the predictive value of the RF_{GFP} and RF_{combined} models. Specifically, for both essentiality screens we used 10 essential genes (all in HEK293FT and the 10 most depleted in A375 cells) and correlated the predicted guide scores from both models to the observed log₂FC guide depletion scores (normalized to 0-100% per gene) of all detected guide RNAs (HEK293FT: n=60 with 6 guides per gene; A375: n=398 with up to 40 guides per gene). We made the same comparison on a per-gene level using all 40 guide RNAs per gene in the A375 screen.

M. Data Representation

In all boxplots, boxes indicate the median and interquartile ranges, with whiskers indicating either 1.5 times the interquartile range, or the most extreme data point outside the 1.5-fold interquartile. All transfection experiments show the mean of three replicate experiments with individual replicates plotted as points.

N. Data Availability Statement

Screen data are being deposited to GEO with an accession number pending. All code and software to reproduce our entire analyses are available on our gitlab repository (gitlab.com/sanjanalab/cas13 ). Moreover, we provide pre-computed guide RNA predictions targeting all protein-coding transcripts in the human genome on our web-based repository (cas13design.nygenome.org). Other data and materials that support the findings of this research are available from the corresponding author upon reasonable request.

O. Code Availability Statement

The predictive on-target model as well as all code for the presented and additional quality control analysis is available on gitlab repository (gitlab.com/sanjanalab/cas13 ).

### Example 4: Principles for Rational Cas13D Guide Design

To date, three different Cas13 effector proteins (PguCas13b, PspCas13b, RfxCas13d) have been reported to show high RNA knock-down efficacy with minimal off-target activity 16,20 We compared the ability of these three Cas13 enzymes to knock-down GFP mRNA when directed to either the cytosol or the nucleus. RfxCas13d (CasRx) consistently showed the strongest target knock-down, especially when fused to a nuclear localization sequence (NLS) data not shown. Using Cas13d-NLS, we varied the guide length while maintaining a constant guide RNA 5′ end or 3′ end relative to a 30 nt reference guide. In both experiments, we found the most pronounced target knock-down using guide RNAs with a length of 23-30 nt (FIG. 4a ). Structural analysis of another Cas13d variant (EsCas13d, PDB: 6E9E/6E9F) suggested that guide RNAs longer than 20 nt extend outside the effector protein binding cleft and that 22 nt guide RNAs provide optimal knock-down²⁸. However, additional guide RNA-target hybridization up to 30 nt in total does not impair target knock-down.

To systematically assess the RfxCas13d target knock-down efficacy of thousands of guide RNAs, we established a monoclonal HEK293 cell line expressing destabilized GFP and doxycycline-inducible Cas13d protein. We lentivirally delivered a library of 7,500 crRNAs that target the GFP coding sequence, containing perfect match and mismatch guide RNAs (FIG. 1a ). We performed fluorescence activated cell sorting (FACS) to gate cells in four bins based on their GFP intensity (FIG. 4a ). Guide counts showed high concordance between bins across three independent transductions with clear separation of bin 1, which contained cells with the lowest GFP expression (FIGS. 4b-4d ).

We calculated the log₂ fold-change (log₂FC) crRNA enrichment between all bins and the unsorted input guide RNA distribution (data not shown). Perfect match guide RNAs were enriched in bin 1, while increasing numbers of mismatches led to a gradual decrease in guide enrichment (FIG. 1b ). This was true for the whole crRNA population as well as for individual guides and their corresponding guides with 1-3 mismatches (FIGS. 1b, 1c). As a control, the library also contained 537 non-targeting crRNAs and they were effectively depleted from bin 1 (FIG. 1b ). As expected, guide abundances in bin 1 were negatively correlated to those in bins 2 to 4, which contained cells with higher GFP intensities. Taken together, this suggests that the enrichments of guide RNAs in bin 1 accurately reflect target mRNA knock-down.

We noticed considerable heterogeneity of guide enrichment within each class (FIGS. 1b-1c). By examining perfect match guide RNAs along the target mRNA, we observed a position-dependent effect, suggesting an influence of the target sequence context on the guide RNA efficacy (FIG. 1d ). We selected 6 guides along the GFP target transcript with either high or low enrichment and validated their relative target knock-down efficacies by transfection of individual guides followed by FACS readout (FIG. 1e ).

To examine if Cas13 can tolerate mismatches between the guide RNA and the target RNA, we calculated the relative log₂ fold change (A log₂FC) for each mismatch guide by subtracting the log₂FC from the reference (perfect match) guide (FIG. 10. We found a critical (“seed”) region for Cas13d knock-down efficacy between guide RNA nucleotides 15 to 21 with its center at nucleotide 18 relative to the guide RNA 5′ end. Although seed regions have been shown for Cas13a orthologs^{12,33,34}, one group reported no clear seed region for Cas13d²⁸ while another group showed position-dependent mismatch sensitivity for Cas13d in a cell-free assay³⁰. Within the seed region, single mismatches led to diminished guide enrichment, while mismatches outside the seed region were better tolerated (FIG. 1f ). The critical region was present irrespective of the mismatch identity (FIG. 1g ). We determined that the decrease in targeting efficacy (delta log₂FC) of crRNAs with single nucleotide mismatches, consecutive double mismatches, random double mismatches and consecutive triple mismatches relative to their cognate perfectly matching guides stratified by mismatch position (two-sided t-test of log₂FC values of permutated guide RNAs versus perfect match guide RNAs. Significance levels: * p<0.05, ** p<0.01, *** p<0.001 ). We also assessed the delta log₂FC in targeting efficacy for random double (RD) mismatches and determined the relative change of targeting efficacy summarized by the number of mismatches falling into the seed region (positions 15 to 21 ).

For randomly distributed double mismatches, the largest change in enrichment was observed in cases where both mismatches are in the seed region. Increasing the number of mismatches to three largely abrogated target knock-down. For this reason, the critical region may have been masked in previous studies on RfxCas13d which used four consecutive mismatches²⁸.

Given the heterogeneity in enrichment for guide RNAs that have mismatches in the seed region, we sought to assess the effect of surrounding nucleotide context. We used partial correlation to control for the knock-down efficacy of the cognate perfect match guide (“reference”), as poorly performing crRNAs might not allow for large changes in enrichment. Controlling for the reference crRNA efficacy, mismatches in a ‘U’-context in the target site negatively impact Cas13d activity, whereas mismatches in a ‘GC’-context were better tolerated. We confirmed the presence of the seed region in transfection experiments using guides with single or double nucleotide mismatches to the GFP mRNA (FIG. 1h ). A single mismatch at guide position 18 led to a marked decrease in knock-down efficacy relative to a perfect match guide RNAs. While a perfect match guide decreased the percentage of GFP-positive cells to ˜29%, a single mismatch at guide position 18 resulted in 75% GFP-positive cells and a double mismatch at positions 17 and 18 resulted in ˜79% GFP positive cells (FIG. 1h ).

Importantly, the center of the RfxCas13d seed region coincides with conserved contacts between a helical domain in Cas13d protein and the backbone of the guide RNA-target hybrid interface. This interaction resides opposite of the guide RNA position 17-18 with the target RNA²⁸. The helical domain is located between both HEPN-domains needed for target cleavage, and mutation of the interacting amino acids in EsCas13d completely abolished target cleavage 28. Mismatches at the seed center thus may impair HEPN-domain activity.

Next we sought to assess the features that may affect knock-down efficacy for perfect match guide RNAs (see Example 6 for details). One of the features impacting the observed guide RNA enrichments in the GFP tiling screen was crRNA folding: Predicting secondary structures and corresponding minimum free energy (MFE) of perfect match crRNAs showed a positive correlation between the MFE and guide efficacy (FIG. 5a ). In particular, ‘G’-dependent structures, such as predicted G-quadruplexes, showed diminished target knock-down. Given that the crRNA folding is critical for effective target knock-down, we sought to further stabilize and improve the DR by repairing a predicted bulge in the DR, by varying the length of the stem loop or by disrupting bases in the proximal DR stem (FIG. 5b ). Analysis of the crystal structure of EsCas13d and UrCas13d together with its crRNA suggested that the terminal loop in the DR may not be embedded within the protein and thus may allow extension (and further stabilization) of the stem loop^{28,30} similar to those previously found to enhance Cas9 activity or utility^{31,32}. We observed that any change in stem length abrogated target knock-down completely (FIG. 5c ). Also, repairing the bulged nucleotide within the stem loop decreased target knock-down. However, disrupting the first base pair within the proximal stem further increased Cas13d targeting efficacy, leading to a novel RfxCas13d DR with improved knock-down capability. We tested the modified DR on 6 additional guides targeting GFP and found that the modified DR improved target knock-down especially for guides with low knock-down efficiency (FIG. 5d ).

We defined 15 crRNA and target-RNA features based on their correlation with observed guide enrichment in our exploratory data analysis (Table 6, Example 6 and data not shown). With these features, we sought to derive a generalizable ‘on-target’ model to predict Cas13d target knock-down. We compared the ability of machine learning approaches to predict guide efficiency (observed log₂FC) in the held-out data (see Methods) and found that a Random Forest (RF) model had the best prediction accuracy (FIG. 10a ), weighting the crRNA folding energy, the local target ‘C’-context, and the upstream target ‘U’-context as the most important features (FIG. 6b ). Other learning approaches frequently chose similar features, suggesting that these features are the main drivers of Cas13d GFP knock-down (FIG. 6c ). To identify key predictor of guide efficiency, we iteratively reduced the number of features, monitoring the model performance and derived a minimal model that explained about 37% of the variance (r² ) with a Spearman correlation (rₛ ) of ˜0.58 to the held-out data (FIGS. 2a and 6d ). In comparison, an support vector machine (SVM) regression model with a similar structure to a Cas9 guide prediction algorithm²⁶ performed worse when applied to this data (r²=0.21, rₛ=0.44 ) (FIG. 2a ). We used 10-fold cross-validation to confirm that the model can readily separate poor performing guide RNAs from effective crRNAs. Accordingly, 46% of the guides present in the highest efficacy-quartile are predicted to reside in the best performing quartile. Conversely, 64% of guides present in the lowest efficacy-quartile are predicted to reside in the poorest performing quartile (FIG. 6e ). Similarly, the predicted standardized guide score of the N top- or bottom-ranked crRNAs confirmed that the model can effectively separate crRNAs that perform well from those that perform poorly (FIG. 6f ).

To show that our model is generalizable, we predicted guides to target the endogenous transcripts of CD46 and CD71, which encode cell surface proteins, and measured the guide knock-down efficacy by FACS. For each gene, we chose 3 guide RNAs predicted to have high knock-down efficacy (Q3 or Q4 ) and 3 guide RNAs predicted to have low knock-down efficacy (Q1 or Q2 ). On an individual guide level, we found that the majority of guides with higher predicted guide scores suppressed CD46 and CD71 protein expression more robustly than guides with lower guide scores (FIG. 2b ). Comparing the observed knock-down across all three high-scoring to all three low-scoring guide RNAs, we found a significant improvement for CD71, while for CD46 we observed considerable variance. To increase throughput and test guide RNA efficacy predictions for more genes, we first generated a small crRNA library targeting 10 essential and 10 control genes with both 3 high-scoring and 3 low-scoring guide RNAs and monitored their depletion in a gene essentiality screen over time. Essential genes were chosen from genes that were strongly depleted in previous RNAi screens³⁷ (FIG. 7a ). Most high-scoring guides targeting essential genes were progressively depleted over time, while low-scoring guides showed largely no depletion (FIGS. 2c and 7b ).

In addition, we performed a second targeted essentiality screen in A375 cells targeting 35 essential and 65 control genes with both 20 high-scoring and 20 low-scoring guide RNAs. Similar to the HEK293 screen above, we found that high-scoring guides that target essential genes were progressively depleted over time (FIGS. 2d and 7c ). Although high-scoring guide RNAs were generally more depleted than low-scoring guide RNAs on a per gene level, we noticed that not all predicted essential genes showed depletion upon Cas13d targeting (FIGS. 7c-7d ), suggesting that RNAi-screen derived essentiality scores may not be one-to-one transferable to Cas13d derived essentiality.

We calculated a significance score of gene depletion based on the guide rank consistency for the 20 high-scoring guides and found strong enrichment of defined essential genes at the top of the list (FIG. 2e ). The guide RNA depletion scores correlated better with the DEMETER2 RNAi⁴⁰ scores used to define the set of essential genes to be tested (up to rₛ=0.71 using the best guide) than with the Cas9 STARS scores¹⁵ (up to rₛ=0.61 ) (FIG. 2f ). Taken together, this suggest that the crRNA and target RNA features derived from the GFP tiling screen can generalize to predict Cas13d guide efficacy for novel targets, and that these guide predictions can be used in pooled CRISPR-Cas13 screens.

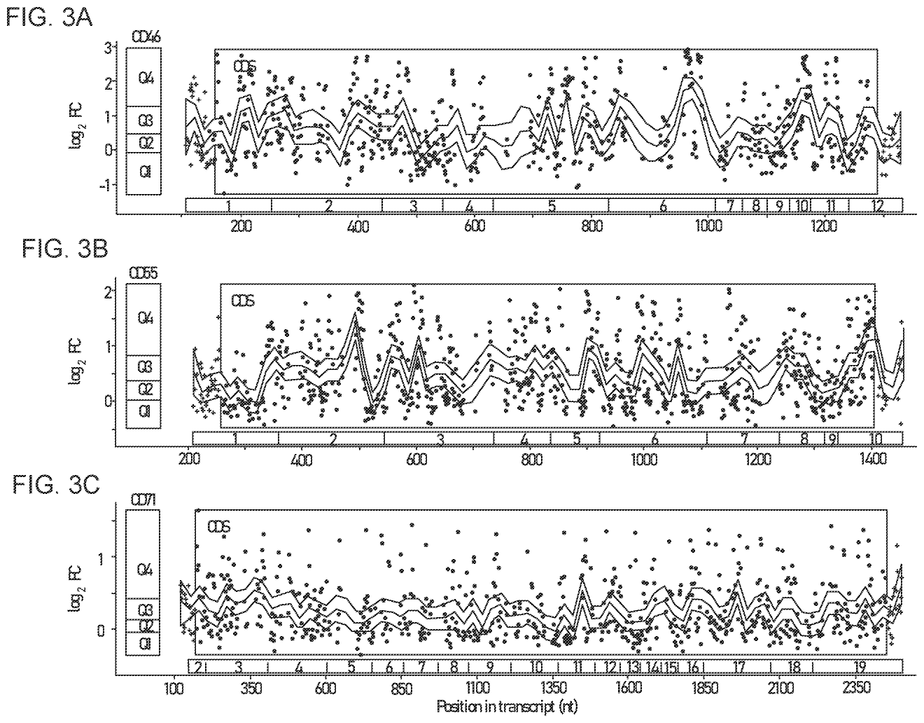

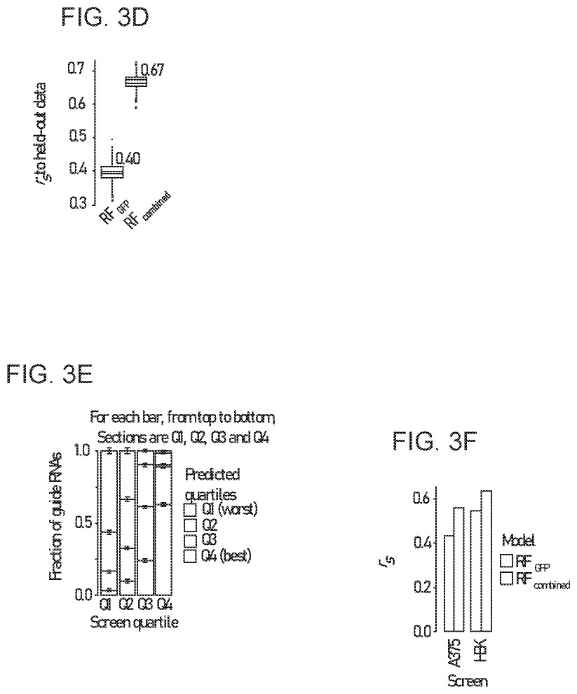

Our predictive on-target model based on the GFP tiling screen was largely able to separate guide RNAs with low knock-down efficiency from those with high efficiency. However, given that we observed remaining heterogeneity among the predicted high-scoring guides, we sought to improve our on-target model by enlarging our training dataset. Therefore, we performed three additional Cas13d tiling screens targeting the main transcript isoforms of the cell surface proteins CD46, CD55 and CD71 in HEK293 cells coupled with FACS readout selecting for cells with decreased surface protein expression (FIGS. 3a-3c ). Besides perfect match guide RNAs, we added several guide RNA classes, including reverse complement (RC), intronic (I), length variants (LV) double mismatch (RD), single mismatch (SM), perfect match (PM) and non-targeting (NT). Together, the three libraries (CD46, CD55, CD71 ) contained 6,069 guide RNAs that were perfectly-matched, 99 guide RNAs with a single mismatch at each of the 23 guide positions (n=2,277 guides), 42 guide RNAs with 100 random double mismatches (n=4,200 guides), and 30 guides with 10 additional guide length variants (n=450 guides), a set of 2188 intron targeting guides centered around the splice-donor and acceptor sites across 40 introns and 300 reverse complement perfect match guide RNAs as additional negative control. Details are not shown here.

CrRNAs are lentivirally transduced into TetO-RfxCas13d HEK293 cells. Five to ten days after transduction, cells are stained for the targeted cell surface protein and sorted by intensities into 2 bins (Bottom 20%=strongest knock-down, Top 20%=least knock-down). For each screen, perfect match guide RNAs showed the strongest guide enrichment relative to the unsorted input samples, while reverse complement negative control guides and non-targeting guides were depleted, data not shown. In the new screens we reduced the overall guide length to 23 bases and included a set of guide length variants ranging in length from 15 to 36 nucleotides. Starting from 23 nucleotide length, guides RNAs exerted full knock-down efficiency, while longer guide 3′ends did not have any deleterious effects.

Perfect match guides targeting coding regions (CDS) were more strongly enriched compared to guides targeting untranslated regions (UTRs) or introns. UTR-targeting guides may show lower enrichments as each target gene may be represented by multiple transcript isoforms with alternative UTR usage. Hence, guides targeting coding regions have a higher likelihood to find the cognate target site while, for example, 3′ UTR-targeting guide RNAs find their target site only in a fraction of the expressed transcripts isoforms. Accordingly, the low enrichment for intron-targeting guide RNAs may be explained by the short-lived nature of introns. For these guides, the intronic target site is present only for a short period of time, which likely enables the transcript to evade Cas13 targeting. For this reason, guide RNA knock-down efficiency may not be directly comparable between CDS-targeting guides and UTR- or intron-targeting guides.

We also observed a slight decrease in guide efficiency of intron-targeting guides immediately downstream of the 5′-splice-site and within the −50 to 0 nucleotide upstream of the 3′-splice-site summarizing across all 39 introns present-. These sites are typically bound by the spliceosome⁴¹, suggesting that guide RNAs targeting these regions may compete with the splice machinery and other splice factors for the target sequences. As transcript maturation in the nucleus seemingly influences the guide RNA targeting efficiency, we wondered if the exon-junction-complex (EJC) would affect knock-down of the matured transcript in the same way. The EJC typically binds ˜20-24 nucleotides 5′ upstream to the exon-exon-junction upon splicing^{42,43}. Indeed, we observed a depletion of high-scoring guide RNAs within a window of −20 to 0 nucleotides 5′ upstream to the exon junction.

To improve our on-target model, we focused on perfect match guide RNAs that target CDS-regions and increased the number of high-confidence model input observation from ˜400 to nearly 3000. We performed a grid-search correlating the observed guide RNA efficacies with the target nucleotide probabilities across a window of 1 nt up to 50 nt at every point 75 nt upstream to 75 nt downstream relative to all 2,918 selected CDS-targeting perfect match target sites. We determined for each nucleotide (A,C,G,U, A|U, G|C) the position and widow size of minimal and maximal Pearson correlation coefficient (Table 7 ). Patterns derived from partial correlation controlling for the crRNA MFE did not deviate from correlations shown. A comparison of machine leaning regression approaches to predict target knock-down of held-out data using bootstrapping was also performed. The data of all CDS-targeting perfect match guides (n=2,918 ) from the all four tiling screens and features (Table 7 ) was randomly split into 70% training data and 30% held-out testing data for 1000 random non-redundant splits. The prediction accuracy (comparing predicted scores to the known log₂FC) is computed using the Spearman correlation (rₛ ) to the held-out data. Models were ranked by their median performance.

Similar to the initial GFP-screen, guide RNAs efficiencies were distributed along the coding region in a non-random manner (FIGS. 3a-3c ). We repeated the assessment of features that may affect knock-down efficacy (see Example 7 for details). Notably, the increased number of observations uncovered positional nucleotide preferences. Guide enrichments correlated positively with G- and C-base probabilities in the seed region around guide position 18. And surrounding this region U- and A-base probabilities correlate positively with the target knock-down. We derived an updated on-target model using 2,918 CDS-targeting guide RNAs across all four tiling screens and selected 35 out of 644 evaluated features in a similar fashion as before (see Example 3, Table 7, Example 7 and data not shown).

The combined Random Forest model (RF_{combined} ) displayed improved prediction accuracy compared to the initial RFₘᵢₙᵢₘₐₗ model (referred to herein as RFG_{FP} ) explaining ˜47% of the variance (r² ) with a Spearman correlation (rₛ ) of ˜0.67 to the held-out data (FIG. 3d ). Using 10-fold cross-validation the model effectively separated low-scoring guides from high-scoring guides, assigning 63% of the guide RNAs correctly to the highest efficacy-quartile (FIG. 3e ). Similarly, the predicted guide scores of the top- or bottom-ranked guide RNAs (ranked by the observed knock-down efficiency) separate guides that performed well from those that performed poorly more than expected by chance. Further, we performed leave-one-out cross-validation training on three data sets while predicting guide scores for the held-out fourth screen. The RF_{combined} model generalized well for endogenous genes (mean±sd: rₛ=0.63±0.01 ) but was less predictive for the GFP transgene (rₛ=0.33 ), data not shown.

Finally, we compared the ability of both models, the RF_{GFP} and RF_{combined} model, with respect to their ability to correctly predict the knockdown efficiencies for the two essentiality screens. Both screens were designed based on guide predictions made by the RF_{GFP} model. In both cases, the RF_{combined} was in better agreement with the observed knock-down efficiencies across all genes (FIG. 3f ). Likewise, we found that the RF_{combined} showed improved agreement with the observed guide RNA depletion also on a gene level for the 10 most depleted genes in the A375 fitness screen (RFG_{FP}: rₛ=0.46±0.16, RF_{combined}: 0.58±0.14 ). Taken together, we show that our updated RF_{combined} on-target model is able to predict Cas13d guide RNA target knock-down efficiencies, separating poorly performing guides from guides with high efficacy and generalized across numerous targets.

We applied our model and predicted guide RNAs for all protein-coding transcripts in the human genome (GENCODE v19 ). We made these predictions available through a user-friendly, web-based application (cas13design.nygenome.org). In addition, we report the 10 highest-scoring crRNAs for the 5′ UTR, CDS and 3′ UTR of each transcript (data not shown). We partitioned the predicted guide RNAs according to the efficacy quartiles in our four screens. Only 15.2% of all possible guides fall into the highest scoring (best knock-down) quartile (Q4 ). A large fraction of guide RNAs are predicted to have lower efficacy (36.8% of all guides are in Q1 or Q2 ), which emphasizes the value of optimal guide selection for high knock-down efficacy. However, almost all transcripts have top-scoring guide predictions.

Taken together, we performed a set of pooled screens for CRISPR Type VI Cas13d and defined targeting rules for optimal guide design. We show that crRNA choice and target RNA-context constrain target knock-down efficacy and, using this data, we develop and validate an ‘on-target’ model to predict guides with high efficacy. Although we specifically sought to define rules for active Cas13d, we believe that our model may be transferable to inactive (catalytically dead) Cas13d effector proteins. Beyond our on-target guide design, we identified a critical seed region in the crRNA that is sensitive to target mismatch. We provide evidence that this seed region can be used in living cells to discriminate between target RNAs with high similarity, such as allele-specific single nucleotide polymorphisms.

### Example 5: Additional Information

TABLE 6
Input features for GFP ‘on-target’ model selection.

| | Feature Name | Description |
|---|---|---|
| 1 | crRNA MFE | Minimum free energy value of DR-sequence plus guide sequence |
| | | using RNAfold |
| 2 | direct repeat | Binary - based on the presence of the predicted “(((((.((( . . . ))).)))))” |
| | | at the crRNA start |
| 3 | G-quadruplex | Binary - based on the presence of the predicted G-quadruplex |
| | | indicated by “+” within the folding sequence |
| 4 | hybMFE 1:26 | Minimum free energy value between guide RNA nucleotides 1-26 |
| | | and its corresponding target site (=overall hybridization) |
| 5 | hybMFE 1:10 | Minimum free energy value between guide RNA nucleotides 1-10 |
| | | and its corresponding target site (=5′ hybridization) |
| 6 | hybMFE 19:8 | Minimum free energy value between guide RNA nucleotides 19-27 |
| | | and its corresponding target site (=3′ hybridization) |
| 7 | log₁₀(Unpaired | log₁₀(probability) of a target RNA nucleotide being unpaired in a |
| | prob1) | window centered at nt −23 relative to the guide match start |
| | | summarizing 21 nts (nt −13:−33) |
| 8 | log₁₀(Unpaired | log₁₀(probability) of a target RNA nucleotide being unpaired in a |
| | prob2) | window centered at nt −23 relative to the guide match start |
| | | summarizing 10 nts (nt −27:−18) |
| 9 | A1 context | Probability of target RNA A-bases at position −22 relative to the |
| | | guide match start summarizing 7 nts (nt −19:−25) |
| 10 | A2 context | Probability of target RNA A-bases at position −22 relative to the |
| | | guide match start summarizing 33 nts (nt −6:−48) |
| 11 | A3 context | Probability of target RNA A-bases at position −16 relative to the |
| | | guide match start summarizing 20 nts (nt −25:−6) |
| 12 | C context | Probability of target RNA C-bases at position −11 relative to the |
| | | guide match start summarizing 22 nts (nt −21:0) |
| 13 | G context | Probability of target RNA G-bases at position −10 relative to the |
| | | guide match start summarizing 21 nts (nt 20:0) |
| 14 | U context | Probability of target RNA U-bases at position −3 relative to the |
| | | guide match start summarizing 18 nts (nt −12:+5) |
| 15 | upstream U | Probability of target RNA U-bases at position −78 relative to the |
| | context | guide match start summarizing 30 nts (nt −93:−64) |

For the RF_{GFP} model we define features as follows: For guide RNA features (features 4, 5, 6 ), nucleotide 1 defines the guide start site (GSS) being the most 5′ guide RNA base matching the target RNA. Nucleotide 2 relative to GSS is the subsequent base (moving in the 5′ to 3′ direction) in the guide RNA and so on. For target RNA features (features 7-15 ), we denote the target nucleotide opposite to the GSS as nucleotide 0. Moving in 5′ to 3′ direction target RNA nucleotide −1 is upstream (5′) to target RNA nucleotide 0 and base-paired to guide nucleotide 2, while target RNA nucleotide +1 is downstream of the target site and so on. A complete illustration for features 4-15 with a schematic of the guide RNA and target RNA can be found in Example 6 and FIG. 9.

TABLE 7
Selected/Extended Input features for RF_{combined} 'on-target' model.

| | Correlation | Feature Name | Description |
|---|---|---|---|
| 1 | Positive | crRNA MFE | Minimum free energy value of DR-sequence plus |
| | | | guide sequence using RNAfold |
| 2 | Positive | Direct repeat | Binary - based on the presence of the predicted |
| | | | “(((((.((( . . . ))).)))))” at the crRNA start (or as listed |
| | | | in CLAIM 19) |
| 3 | Negative | G-quadruplex | Binary - based on the presence of the predicted G- |
| | | | quadruplex indicated by “+” within the folding |
| | | | sequence |
| 4 | Positive | HybMFE 3:15 | Minimum free energy value between guide RNA |
| | | | nucleotides 3-15 (or a sequence including |
| | | | nucleotide 15-23) and its corresponding target site |
| | | | (=5′ hybridization) |
| 5 | Negative | HybMFE 15:23 | Minimum free energy value between guide RNA |
| | | | nucleotides 15-23 (or a sequence including |
| | | | nucleotide 15-23) and its corresponding target site |
| | | | (=3′ hybridization) |
| 6 | Positive | Log₁₀(Unpaired | log₁₀(probability) of a target RNA nucleotide being |
| | | prob) | unpaired in a window centered at nt −11 relative to |
| | | | the guide match start summarizing 23 nts (nt 0:−22) |
| | | | (or a sequence including nucleotide 0:−22) |
| 7 | Positive | Local Aₘₐₓ | Probability/Proportion of target RNA A-bases at |
| | | probability | position −10 relative to the guide match start |
| | | | summarizing 8 nts (nt −14:−7) (or a sequence |
| | | | including nucleotide 14:−7) |
| 8 | Positive | Local Cₘₐₓ | Probability/Proportion of target RNA C-bases at |
| | | probability | position −16 relative to the guide match start |
| | | | summarizing 4 nts (nt −18:−15) (or a sequence |
| | | | including nucleotide 18:−15) |
| 9 | Positive | Local Gₘₐₓ | Probability/Proportion of target RNA G-bases at |
| | | probability | position −19 relative to the guide match start |
| | | | summarizing 3 nts (nt −20:−18) (or a sequence |
| | | | including nucleotide 20:−18 |
| 10 | Positive | Local Uₘₐₓ | Probability/Proportion of target RNA U-bases at |
| | | probability | position −6 relative to the guide match start |
| | | | summarizing 12 nts (nt −12:−1) (or a sequence |
| | | | including nucleotide nt −12:−1) |
| 11 | Positive | Local AUₘₐₓ | Probability of target RNA A or U-bases at position −7 |
| | | probability | relative to the guide match start summarizing 11 |
| | | | nts (nt −12:−2) (or a sequence including nucleotide |
| | | | nt −12:−2) |
| 12 | Positive | Local GCₘₐₓ | Probability/Proportion of target RNA G or C-bases |
| | | probability | at position −18 relative to the guide match start |
| | | | summarizing 9 nts (nt −22:14) (or a sequence |
| | | | including nucleotide nt 22:14) |
| 13 | Negative | Local Aₘᵢₙ | Probability/Proportion of target RNA A-bases at |
| | | probability | position −17 relative to the guide match start |
| | | | summarizing 7 nts (nt −20:−14) (or a sequence |
| | | | including nucleotide nt 20:−14) |
| 14 | Negative | Local Cₘᵢₙ | Probability/Proportion of target RNA C-bases at |
| | | probability | position −3 relative to the guide match start |
| | | | summarizing 9 nts (nt −7:+1) (or a sequence |
| | | | including nucleotide nt −7:+1) |
| 15 | Negative | Local Gₘᵢₙ | Probability/Proportion of target RNA G-bases at |
| | | probability | position −9 relative to the guide match start |
| | | | summarizing 9 nts (nt −13:−5) (or a sequence |
| | | | including nucleotide nt −13:−5) |
| 16 | Negative | Local Uₘᵢₙ | Probability/Proportion of target RNA U-bases at |
| | | probability | position −17 relative to the guide match start |
| | | | summarizing 10 nts (nt −22:−13) (or a sequence |
| | | | including nucleotide nt 22:−13) |
| 17 | Negative | Local AUₘᵢₙ | Probability/Proportion of target RNA A or U-bases |
| | | probability | at position −17 relative to the guide match start |
| | | | summarizing 9 nts (nt −21:−13) (or a sequence |
| | | | including nucleotide nt 21:−13) |
| 18 | Negative | Local GCₘᵢₙ | Probability/Proportion of target RNA G or C-bases |
| | | probability | at position −18 relative to the guide match start |
| | | | summarizing 11 nts (nt −11:−1) (GCₘᵢₙ - not used in |
| | | | RF_{combined}) |
| 19- | Positive | Nucleotide | Probability/Proportion of guide RNA A, C, G or U |
| 22 | | probability | bases (U - not used in RF_{combined}) |
| 23- | Positive | Di-nucleotide | Probability/Proportion of 16 possible guide RNA |
| 39 | | probability | di-nucleotides (UU - not used in RF_{combined}) |

For the RF_{combined} model we define features as follows: For guide RNA features (features 4 and 5 ), nucleotide 1 defines the guide start site (GSS) being the most 5′ guide RNA base matching the target RNA. Nucleotide 2 relative to GSS is the subsequent base (moving in the 5′ to 3′ direction) in the guide RNA and so on. For target RNA features (features 6-18 ), we denote the target nucleotide opposite to the GSS as nucleotide 0. Moving in 5′ to 3′ direction target RNA nucleotide −1 is upstream (5′) to target RNA nucleotide 0 and base-paired to guide nucleotide 2, while target RNA nucleotide +1 is downstream of the target site and so on. A complete illustration for features 4-18 with a schematic of the guide RNA and target RNA can be found in Example 7 and FIG. 10.

### Example 6: Features of Cas13D Targeting from the GFP Tiling Screen

A. Anti-Tag

Recently, others have found that Cas13a is inhibited by a 4 nt “anti-tag” sequence homology between the end of the DR and the corresponding flanking sequence of the target and have speculated that Cas13d, which has a similarly positioned 5′ DR, might also use an anti-tag for host versus pathogen discrimination¹⁰. Using all perfect match guide RNAs, we did not find evidence for the presence of a similar anti-tag sequence for RfxCas13d suggesting that anti-tags may not be found in all Type VI CRISPRs or contribute only marginally compared to other features, data not shown.

B. Nucleotide Preferences

Next, we tested whether position-based nucleotide preferences exist within the guide RNA target sequence or nearby nucleotides by comparing the nucleotide composition of the top 20% to all perfectly matching guides in the GFP screen, similar to previous approaches assessing Cas9 guide preferences¹¹. Although the top enriched guides showed slight nucleotide preferences compared to all guides, most preferences became insignificant after correction for multiple hypothesis testing. However, when we correlated guide RNA nucleotide probabilities with the observed guide enrichment, we saw that high ‘G’-content in the guide RNA had a strong negative impact. Other measures, like guide RNA GC-content indicated a local optimum around 50% with lower guide efficiency when the guide adopts lower or higher GC proportions.

C. crRNA Folding

The negative correlation to the observed guide RNA enrichments (log₂FC) was restricted to high ‘G’-content in the guide RNA, while guide RNA ‘C’-content did not affect targeting in the same way. This suggests that the effect may not be caused by specific guide-target interaction, which should weight ‘C’ and ‘G’ bases interchangeably, but instead may be driven by ‘G’-dependent stable structures within the crRNA that may render the crRNA inaccessible for Cas13d. Indeed, predicting the secondary structure and corresponding minimum free energy (MFE) of perfect match guides showed a positive correlation between the MFE and guide efficacy (FIG. 5a ). In particular, ‘G’-dependent structures, such as predicted G-quadruplexes, showed diminished target knock-down.

D. Guide RNA—Target RNA Hybridization

We next tested whether guide-target hybridization can contribute to guide RNA efficacy by computing the correlation between hybridization energy and guide RNA efficacy. The Pearson correlation coefficient (rₚ ) of the observed log₂FC and the hybridization minimum free energy (MFE) of guide RNA nucleotide position p over the distance d to the position p+d with its cognate target sequence for all perfectly matching guide RNAs was determined. For the GFP screen we found that more stable hybridization between guide RNAs and their target sequences (lower MFE) was correlated with lower guide RNA efficacy (r=0.31 ). This suggests that the most stable guide-target interactions may render the ribonucleoprotein complex less active. Interestingly, calculating MFEs between smaller regions within the guide RNA indicated multiple sub-structures that contribute to the overall correlation, suggesting that individual parts of the guide-target interaction may serve specific roles during ternary complex formation or nuclease activation. However, these correlative structures were nearly gone when using partial correlation to control for the effect of crRNA folding.

E. Target Site Nucleotide Context

Beyond guide RNA nucleotide composition, we wondered if the context features of the guide RNA target site affected target knock-down. By correlating the observed guide RNA log₂FC with the nucleotide probabilities across windows around target sites, we detected a strong negative impact of high ‘C’-context directly at the target site. However, this may be confounded by the high guide RNA ‘G’-content and its role in crRNA folding (FIG. 5). Indeed, using partial correlation to account for the crRNA MFE diminished the negative correlation strength. Outside the direct target site, we noticed that high ‘U’-content upstream (5′) to the target site positively correlated with target knock-down, which is consistent with previous reports of higher nuclease activity in ‘U’-rich contexts^{22,38}. In order to understand if the observed upstream U-context is generalizable or targeting position specific, we generated a GFP reporter plasmid that allowed for changing the nucleotide context upstream of a perfect match target site. We designed a 52mer oligonucleotide lacking uridines and optimized to minimize predicted RNA secondary structures. We cloned this and 52mer oligonucleotides with 3 or 6 uridines at various positions into the GFP-reporter plasmid and tested the upstream uridine context effect on target knock-down. Each reporter was targeted directly downstream of the introduced oligo, or with a non-targeting guide. This was done, because the introduced uridines could potentially act as cis-regulatory elements and recruit RNA binding proteins and thus influence target RNA stability independent of the Cas13 protein⁴⁴. We did not observe a significant position dependent effect of the upstream (5′) uridines (data not shown), suggesting that the effect may be target site specific, driven by additional downstream U content or too weak to be assessed in this experiment.

F. Target Site Accessibility

We also assessed whether the target site accessibility influences knock-down by correlating the observed guide RNA efficacies with the target site accessibility. Here, we define target site accessibility as the probability that the target RNA (in this case, GFP mRNA) is unpaired. We found a weak positive correlation with increased target site accessibility centered on the 3′-end of the spacer RNA (FIG. 8) reminiscent of target-RNA accessibility preferences shown for Cas13b

G. On-Target Model Feature Collection

Based on our analyses above, we determined the position and window-size with the best correlation to the observed guide RNA enrichments for each feature (FIG. 9). A full list of all features evaluated in the on-target model based in the GFP-tilling screen data can be found in Table 6.

### Example 7: Features of Cas13D Targeting from Combined Tiling Screens

For the assessment of crRNA and target RNA features, we considered 2,918 perfect match guides that target coding regions across four genes: GFP, CD46, CD55, CD71 (see FIGS. 1d and 3a ). Compared to the 399 perfect-match guide RNAs from the GFP screen alone, this represents a 7.3-fold increase in data points. To make the screens more comparable, we scaled the log₂FC of each screen independently. We preferred scaling each dataset over assigning ranks between 1 and 0 in order to maintain relative guide strength differences between screens, under the assumption that the strongest guide in screen A may not be as strong as the strongest guide in screen B. Here, each feature is represented across all 4 screens. In addition to this combined analysis, analyses for each independent screen are available but not shown here.

A. Anti-Tag

Using all perfect match guide RNAs, we did not find evidence for the presence of an anti-tag sequence²⁷ for RfxCas13d suggesting that anti-tags may not be found in all Type VI CRISPRs or contribute only marginally compared to other features.

B. Nucleotide Preferences

Next, we tested whether position-based nucleotide preferences exist within the guide RNA target sequence or nearby nucleotides by comparing the nucleotide composition of the top 20% to all perfectly matching guides across all four screens, similar to previous approaches assessing Cas9 guide preferences¹¹. The increased number of data points uncovered clear nucleotide preferences. Effect-size (delta nucleotide probabilities) and Bonferroni-corrected p-values of observing the conditional probability of a guide in the top 20% under the null distribution examined at every position including the 4 nucleotides upstream and downstream of the guide RNA target site was determined. The p-values were calculated from the binomial distribution with a baseline probability estimated from the full-length mRNA target sequence all perfect match guide RNAs. The top 20% were selected for each screen separately to ensure equal contribution. The top enriched guides showed preferences for G-bases at guide nucleotides 19-21 (with position 1 defined as the most 5′ nucleotide in the guide RNA that matches the target RNA). C-bases were favored at positions 15-16. Interestingly, the enrichment of G and C bases surround the center of the critical seed region at position 18 (see FIG. 1f ). Moreover, we observed before that increased GC-content surrounding mismatches at position 18 correlated with the relative decrease in guide efficiency (delta log₂FC). It appears that increased high GC-content may ameliorate the effect size of mismatches in the seed region. There was also a mild enrichment for A- and U-bases in the first half of the guide RNA.

We correlated guide RNA nucleotide probabilities with the observed guide enrichment. In the GFP screen data alone we found that high ‘G’-content in the guide RNA had a strong negative impact. This impact was reduced when taking all four screens into account. The guide RNA GC-content indicated a local optimum around 50% with lower guide efficiency when the guide adopts lower or higher GC proportions.

C. crRNA Folding

Analyzing the GFP screen alone, we found previously that the predicted crRNA folding minimum free energy (MFE) of perfect match guides correlated positively with guide RNA efficacy (see FIG. 5a ). Low MFE values were associated with low guide RNA efficiencies suggesting that stable crRNA folds may hinder crRNA utilization by Cas13d. Extending this analysis to perfect match guide RNAs of all screen, we observed an overall decrease in the correlation between crRNA MFE and guide efficiency. However, low MFEs still associated with low guide RNA efficiencies. Additional predicted G-quadruplex structures were not observed in the CD46, CD55 and CD71 screens.

D. Guide RNA—Target RNA Hybridization

We next tested whether guide-target hybridization can contribute to guide RNA efficacy when integrating data from all four screens. Unlike for the GFP screen alone, we found that the overall hybridization energy between the full-length guide RNA and target sequence correlated less. Instead, the hybridization energies of sub-fragments contributed differentially to the overall guide-target interaction. The hybridization energy between the 12 nucleotides from guide position 3 to 15 and the cognate target site showed a slight positive correlation. Hybridization energies covering the 9 nucleotides from guide position 15 to 23 correlated negatively with the knock-down efficiencies. Unlike for the GFP screen analysis before, these sub-structures were still present when controlled for the crRNA folding energies using partial correlations.

E. Target Site Accessibility

We also assessed the target site accessibility for all screens and correlated observed guide RNA efficacies with the target site accessibility. Here, we define target site accessibility as the probability that the target RNA is unpaired. We did not find a strong relationship between the probability of the target sequence being unpaired and the observed knock-down strengths. Similar to the GFP screen alone, we find a weak positive correlation with increased target site accessibility centered on the 3′-end of the spacer RNA. We also recapitulate the observed nucleotide preferences with higher GC-content centered around seed nucleotide 18, which is surrounded by higher AU-content. Higher AU-content translates to increased accessibility, while higher GC content suggest local secondary structures.

F. On-Target Model Feature Collection

Based on our analyses across all four tiling screens, we determined the position and window-size with the best correlation to the observed guide RNA enrichments for each feature (FIG. 10). For the RNA target site accessibility we chose the entire target site as a window instead of the weak positive correlation that correlated with the U-context in in that region (from nucleotide 1-23 with position 1 defined as the most 5′ nucleotide in the guide RNA that matches the target RNA). A full list of all features evaluated in the on-target model based in the GFP-tilling screen data can be found in Table 7.

### Example 8: Summary of Screen Data

To show the overall distribution of GFP signal in response to the screen, the GFP flow cytometry plot in FIG. 4a is presented with an overlay of 1 ) GFP-negative HEK293FT cells, 2 ) untransduced HEK293FT-Cas13d-GFP cells and 3 ) HEK293FT-Cas13d-GFP cells transduced with the GFP-targeting crRNA library. We added several new screens tiling mRNAs of endogenously expressed cell surface receptors and, similarly, added FACS gating strategy figures for the newly-added CD46, CD55 and CD71 tiling screens. All FACS plots for cell-surface receptors include 1 ) unlabeled cells, 2 ) antibody-labeled cells transduced with a pooled library and 3 ) antibody-labeled cells transduced with a non-targeting guide. In all four screens (GFP, CD46, CD55, and CD71 ), the signal distribution shifts lower compared to control cells.

A GFP-FSC scatter plot is also presented in FIG. 4a, which shows that cells of all sizes show depletion in GFP signal and that selection for GFP is not related to selection for size.

Regarding predictions for Bins 2-4: Bins 2, 3 and 4 did not enrich for high-efficiency guide RNAs, but instead were depleted for high-efficiency guide RNAs. However, to clarify, our predictive model does not try to make predictions specifically for Bins 2-4. Rather, the targeting quartiles are based on the guide RNA enrichments within Bin 1 (presented in FIGS. 3e and 6e ). For each screen, Bin 1 represents the bin with the lowest target gene expression (and highest target knock-down). Within Bin 1, we split the guide RNA efficiency in quartiles based on their log₂FC enrichments. Therefore, the prediction quartiles are restricted to predict the guide RNA efficiency within bin 1. To make this connection clearer, guide RNA efficiency quartile labels are indicated in FIGS. 1d and 3a-3c. These labels match the color labels in FIGS. 6e and 3e, respectively.

Regarding the outliers, they may have been introduced during the screen (e.g. FACS selection or PCR amplification) as the outliers do not show any consistency between transduction replicates. In contrast, outliers introduced during transduction would display a guide-specific pattern in input and sorted samples of the same biological (transduction) replicate, which would be apparent in the Pearson correlation coefficients in FIG. 4c. However, we do not see this. If the same guide RNAs had high counts throughout the unsorted input samples and sorted bin, then clustering would happen by replicate. Again, we do not see this.

As this was not the case, we removed single outlier counts for a single biological sample, but not the entire gRNA for the GFP screen (clarified in the methods). Normalization was done including all counts, as all counts contribute to the library size. Moreover, outliers were not enriched for a particular class of guides and are a small minority of the points. Overall, the outlier detection procedure resulted in the removal of ˜2% of data points with the highest residuals across the 15 biological samples. In conclusion, we considered the outlier to be a random confounder and thus masked individual counts only when detected as an outlier. Most importantly, by masking outliers we reduced the number of perfect match guide RNAs used for the initial on-target model by only 1 guide from 400 to 399, and by 4 guide RNAs overall. A table is provided below summarizing reproducibility (correlation) of bins 1, 2, 3, 4 and input counts throughout the normalization steps across the three replicate GFP-screens. A complete set of all pairwise correlations can be found in FIG. 4c. In light of the addition of five more screens to this work, we have made all quality control and data processing of the GFP screen and the additional screens available (data now shown).

TABLE 8
Summary statistics of GFP screen guide count correlations throughout four processing
steps. Each correlation represents the mean +/− standard deviation of three
replicate experiments among the input samples and the indicated bins 1 through 4.

| Spearman correlation | Input | Bin 1 | Bin 2 | Bin 3 | Bin 4 |
|---|---|---|---|---|---|
| 1) raw | 0.79 +/− | 0.84 +/− | 0.8 +/− | 0.79 +/− | 0.81 +/− |
| counts | 0.02 | 0.02 | 0.01 | 0.02 | 0.01 |
| 2) after | 0.79 +/− | 0.84 +/− | 0.8 +/− | 0.79 +/− | 0.81 +/− |
| normalization | 0.02 | 0.02 | 0.01 | 0.02 | 0.01 |
| 3) after | 0.83 +/− | 0.88 +/− | 0.85 +/− | 0.85 +/− | 0.85 +/− |
| batch-correction | 0.01 | 0.01 | 0 | 0.01 | 0.01 |
| 4) after | 0.9 +/− | 0.93 +/− | 0.92 +/− | 0.89 +/− | 0.91 +/− |
| outlier-removal | 0.01 | 0 | 0 | 0.01 | 0.01 |

We initially attempted to compare our predicted scores to guides that have been used in previous Cas13d work. Given the few papers in this field, we had difficulty making a meaningful comparison, as other RfxCas13d papers used only a very small numbers of guide RNAs (e.g Ref 22 ) or a non-mammalian context (e.g. bacterial library in EsCas13d from Ref 17 ).

In light of these issues, we decided to significantly expand our dataset and added three additional tiling screens targeting the human transcripts CD46, CD55 and CD71. This data is in FIG. 3. The additional data led to an improved on-target model compared to the GFP-based model. Moreover, we also added two screens targeting essential and control genes in human kidney cells (HEK293FT) and melanocytes (A375 ). We experimentally evaluated 3,979 new perfectly-matching guide RNAs targeting 48 endogenous target genes in the revised manuscript. Our improved on-target model (RF_{combined} ) predicts correctly 63% of guides in the highest scoring efficiency quartile, whereas our previous model (RFG_{FP} ) achieved only 46% correct assignments. Also, RF_{combined} has better prediction accuracy (rₛ=0.67 ) than commonly used Cas9 on-target models derived from similar tiling screens (2014: rₛ=˜0.45, 2016: rₛ=˜0.5 )^{11,29}.

Indeed, we observed overall high consistency between the samples. One reason for the high consistency may be that the plasmid crRNA libraries showed a very even distribution of guide counts (e.g. comparing the 90^{th} to 10^{th} percentile ratio, we determined a skew from 2.2 or less for all screens present in this work). Compared to previous large-scale Cas9 libraries (e.g. GeCKOv1, Ref 2 ), this is nearly a 10-fold improvement in library uniformity. As a consequence, guide RNAs may likely be represented very evenly in the unsorted input samples. In addition to that, we sequenced the GFP screen to a high depth (˜450 reads per guide). Taken together, this may explain the overall good agreement of guide RNAs (and even ones with low representation/counts) in the GFP screen. No additional filtering (e.g. removal of guide RNAs below a minimum count) was used.

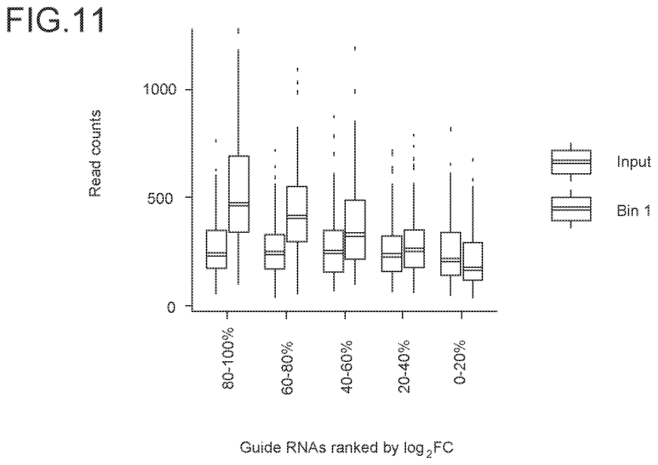

The low read count guides are likely not outliers in other aspects. Specifically, we grouped the perfect match guide RNAs into 5 consecutive bins (20% bins) based on their log₂FC enrichments and compared the associated guide counts in Input and Sort Bin 1 samples (=highest knock-down). We find that all guides are evenly represented cross the entire range of log₂FC enrichments in the input samples (FIG. 11).

The observed effects are driven by the differential guide RNA enrichments in Sort Bin 1. It is important to note that our conclusion is based on the average base probabilities of the bottom 0-20% guides (n=80 guide RNAs).

We tested the modified crRNA direct repeat (DR) for 6 additional guide RNAs. We tested the same 6 guide RNAs presented in FIG. 1e. These 6 guide RNAs included 3 guide RNAs that we previously found to have low knock-down efficiency and 3 guide RNAs that had high knock-down efficiency.

We found that the modified DR improved GFP knock-down for low knock-down efficiency guide RNAs, but the effect was negligible for high knock-down efficiency guide RNAs. Overall, the modified DR seems to either improve knock-down efficiency or have no effect when knock-down is already strong.

In our initial GFP tiling screen, we noticed that the guide RNA activity correlated positively with the uridine probability ˜50 nucleotides upstream of the guide RNA target site via a grid search over positions 75 nt upstream to 75 nt downstream of the target site. This finding is consistent with previous reports of higher nuclease activity in ‘U’-rich contexts^{22,38}. Specifically, Konermann et al. 2018 tested the EsCas13d cleavage activity in single stranded and structured context varying the upstream nucleotide identities. The authors found that target cleavage showed a significant preference for U-bases. The exact distance to the target site was not addressed. In Freije et al. 2019, the authors found that active guide RNAs had a higher U-probability within the 50 nt upstream and downstream of the target site compared to non-active guide RNAs for a Cas13a variant. However, it is not clear if this finding was statistically significant. Similarly, since the correlation between the upstream U content and the observed guide efficiency in our GFP screen was driven by a group of guides that all fell into the same region of the GFP target transcript, we were concerned the observed upstream U-context might not be generalizable (i.e. targeting position specific).

To address this concern, we generated a GFP reporter plasmid that allowed for changing the nucleotide context upstream of a perfect match target site. We designed a 52mer oligonucleotide lacking uridines and optimized to minimize predicted RNA secondary structures. We cloned 52mer oligonucleotides with 3 or 6 uridines at various positions into the GFP-reporter plasmid and tested the upstream uridine context effect on GFP knock-down. Each reporter transcript was targeted directly downstream of the introduced oligo, or with a non-targeting guide. This was done, because the introduced U-stretches could potentially act as cis-regulatory elements and recruit RNA binding proteins and thus influence target RNA stability⁴⁴ independent of the Cas13 protein. We did not observe a significant position-dependent effect of upstream Us (data not shown), suggesting that the effect may be target site specific, driven by additional downstream U content, or too weak to be assessed in this experiment. More importantly, we did not find a similar correlation of U-probability and guide efficiency for the CD46, CD55 and CD71 tiling screens (˜6.3× larger dataset).

The linear combination of nucleotide context (which we term herein as “NT-context+”) represents the following model formula:

guideRNA efficiency (log₂FC)˜local A1+local C+local G+local U+upstreamU+crRNA MFE

Each of the listed model parameters is defined in Table 6. This linear model utilizes the same 6 features from the RF_{GFP} model. Although the features are selected (see next paragraph), the model (NT-context+) itself is just a linear (regression) model.

We identified the features most highly correlated with guide RNA efficacy using an exhaustive (grid) search ranging over 75 nt upstream to 75 nt downstream of the target site. At each position, we determined if the nucleotide (A, C, G, U, GC, AU) probability over a window size of 1 to 50 nt correlated with the observed guide RNA efficiencies (log₂FC). For A nucleotides in the initial GFP screen, we identified three positions (termed A1, A2, and A3 ) with similar Pearson correlation coefficients.

Specifically, these predictive features are:

A1 is the probability of A-bases in a 7 nt window centered at nucleotide 23 relative to the guide sequence start (GSS). We define “nucleotide 1 relative to GSS” as the most 5′ guide RNA base matching the target RNA. “Nucleotide 2 relative to GSS” is the subsequent base (moving in the 5′ to 3′ direction) in the guide RNA and so on.

A2 is the probability of A-bases in a 33 nt window centered at nucleotide 23 relative to the GSS.

A3 is the probability of A-bases in a 20 nt window centered at nucleotide 17 relative to the GSS.

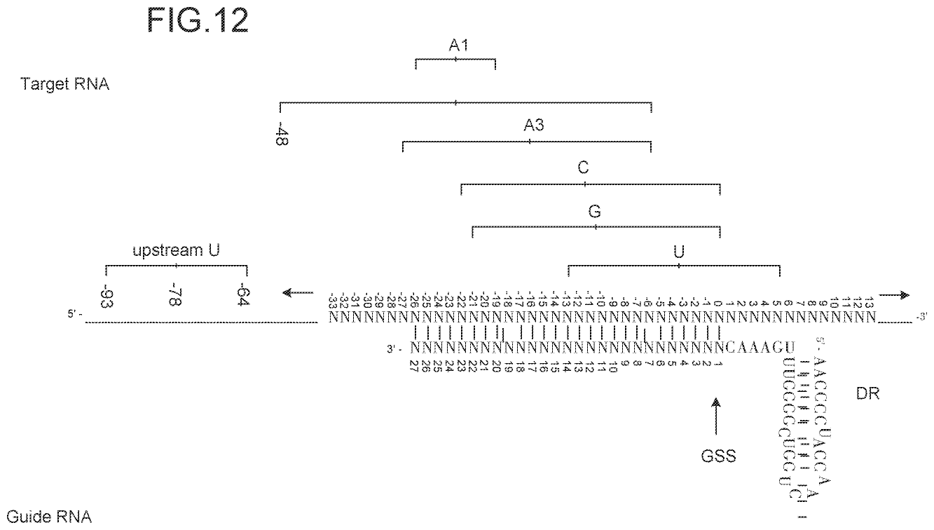

A complete description of all features that have been selected can be found in Tables 6 and 7. We have summarized the selected features for the GFP-screen based model (see FIG. 12).

Naïvely, predicted low-scoring guides should not confer any knock-down, while predicted high-scoring guides should confer strong knock-down. However, we show in FIG. 1e that low-scoring guides that target GFP, are still capable to confer GFP knock-down to some degree. Thus low-scoring guides may either show no or diminished knock-down compared to high-scoring guides. In that sense, it is not surprising that the predicted low-scoring guides can confer CD46 and CD71 knock-down in FIG. 2b.

The shift in the distribution from CD46 and CD71 knock-down (FSC vs. CD46/CD71 ) shows a unimodal distribution (i.e. cells of all sizes are shifting to less CD46 or CD71 signal, respectively).

In our revised study, we conducted several additional screens (in total, a ˜6.3× larger dataset) and built a new predictive Cas13 guide RNA model. In all of these tiling screens (GFP, CD46, CD55, CD71 ), we do not see a specific impact of target position along the transcript.

For example, if we simply compare the ratio of the average knock-down efficiency of guide RNAs in the first 50% of each transcript versus those in the last 50% of each transcript, we did not observe significant differences (mean±s.d.: 0.97±0.08 ). Moreover, we assessed the presence of a correlation between the guide RNA match position and the observed guide RNA efficiency and found no clear connection (mean rₛ=−0.02±0.07 ).

In contrast, specific nucleotide contexts are among the most important features in the improved predictive model (see FIG. 13). A full description of the features used can be found in Tables 6 and 7.

Our main focus in this work was active Cas13 for knock-down of target RNAs. We believe that assessing nuclease-inactive applications of dCas13 (such as the modulation of alternative splicing) requires a different readout from the FACS-based screens described in our work. (This is in contrast to nuclease active Cas9 and inactive dCas9-KRAB applications, which can be assessed by the same readout.) Experiments are under investigation addressing this important point with an appropriate readout for dCas13d, such as an alternative splicing reporter (as shown in Konermann et al. 2018 ).

We greatly improved the initial start time (˜100× faster, <10 seconds). Moreover, we have improved data visualization to plot all predictions along the target transcript and have added new guide RNAs to target both 5′ and 3′ UTRs. We have also made it easy to download gene-specific visualizations and guide RNAs.

Endogenous Targets

We added five screens with endogenous targets. Two of these screens (HEK293FT and A375 gene essentiality screens) aimed to validate our initial on-target model based on the GFP screen by comparing low-scoring and high-scoring guide RNAs head-to-head with higher throughput. And in addition, three tiling screens (CD46, CD55, CD71 ) aimed to enlarge the dataset for training an on-target model. In total we added measurements for 21,763 additional guide RNAs to the initial set of 7,500 GFP-targeting guide RNAs. Considering only endogenous genes, we have evaluated our on-target models on 48 endogenous target genes (essential genes plus cell surface proteins) and 3.979 coding sequence targeting perfect match guide RNAs.

Significance Testing

For 47 genes we expected detectable changes when targeted with either low- or high-scoring guide RNAs (2 genes in FIG. 2b; 10 essential genes in FIG. 7b, and 35 essential genes in FIG. 7d ). For each gene, we included a one-tailed t-test to calculate the probability of high-scoring guide RNAs conferring stronger target knock-down compared to low-scoring guides (FIGS. 2b, 7b and 7d ). For 29 genes tested, we found that high-scoring guide RNAs led to significantly stronger target knock-down (guide depletion) than low-scoring guide RNAs. For 8 genes the differences between predicted low-scoring and high-scoring and the observed changes was not significant. In none of these 37 cases we found predicted low-scoring guides to perform better than predicted high-scoring guides. For 10 essential genes (FIG. 7d, the 10 right-most genes), we could not observe guide RNA depletion relative to input guide RNA counts.

To more globally compare gene essentiality between RNAi, CRISPR-Cas9 and Cas13d, we derived a p-value for the relative guide depletion in the A375 screen, under the assumption that more essential genes would show more pronounced guide depletion. Specifically, we calculated a p-value based on the log₂FC rank consistency of the most depleted N guides (N in {1, 5, 20}) using robust rank aggregation (RRA)³⁹. RRA assesses the relative rank of each group of selected guides across all 100 genes present in the A375 screen. In this way, RRA represents a multiple-comparisons test in which the consistency of relative guide ranks is compared across genes. The outcome represents a p-value for gene essentiality under the null hypothesis that there are no essential genes (i.e. that there are no guides that rank robustly at the top of the ranked essentiality list). Using all 20 high-scoring guides per gene, we found that essential genes were associated with lower p-values and separate clearly from control genes (FIG. 2e ).

Moreover, we used the derived p-value (Cas13 essentiality score) and compared our score to Cas9 and RNAi derived gene essentiality scores in A375 cells. For this comparison we used non-parametric Spearman rank correlations of all genes in our A375 dataset with essentiality scores in all three approaches. All 35 essential genes were included along with 15 control genes. The -log₁₀p-values (Cas13 essentiality scores) correlated better with the DEMETER2 RNAi⁴⁰ scores (up to rₛ=0.71 ) than with the Cas9 STARS scores¹⁵ (up to rₛ=0.61 ) (FIG. 20. In comparison, McFarland et al. reported that the DEMETER2 RNAi scores compare to Cas9-CERES scores globally (for most genes and across 391 cell lines) with a Pearson correlation coefficient of r=0.58⁴⁰.

Targeting Complex Transcript Features (UTRs and Introns)

Beyond our efforts to validate the initial on-target model based on the GFP screen data, we conducted three additional tiling screens targeting genes that encode for cell surface proteins (CD46, CD55 and CD71 ). These new tiling screens enabled us to assess features we could not assess using the GFP screen alone. For example, we found that guide RNAs targeting coding sequences showed overall stronger enrichments (target depletion) compared to guide RNAs targeting untranslated regions (UTRs) or introns.

This observation may be explained in part by differential target-site availability. Intronic sequences are comparably short-lived and thus can be targeted only for a short period of time during the lifespan of the target transcript. 3′UTRs on the other hand may undergo differential cleavage and polyadenylation, hence only a fraction of transcripts will contain guide RNA target-sites that target longer 3′UTRs. However, data from 3′UTR-end sequencing by Christine Mayr's lab¹⁸ suggests that CD55 shows strong evidence for alternative cleavage and polyadenylation in HEK293FT cells, while CD46 and CD71 may only express one 3′UTR isoform. Nevertheless, all three target genes show the same enrichment pattern: CDS>5′ UTR≈3′ UTR>introns, in order of largest median fold-change to smallest median fold-change.

Complex Transcript Features: Splice Junctions

Using a set of ˜2100 intron-targeting guide RNAs across all 39 introns (CD46 n=12 introns, CD55 n=9 introns, CD71 n=18 introns), we found a decrease in guide efficiency of intron-targeting guides immediately downstream of the 5′-splice-site and within the −50 to 0 nucleotide upstream of the 3′-splice-site. The Ule lab recently showed that these sites are usually bound by proteins of the spliceosome¹⁹, suggesting that Cas13d may compete with splicing factors and other RNA-binding proteins³⁸ for intronic target sequences. Similarly, we found a decrease of strongly enriched guide RNAs −20 to 0 nucleotides upstream of exon-exon-junctions, which may indicate that Cas13 competes with the exon-junction-complex for target-sites directly upstream (20-24 nt) of exon-exon-junctions^{20,21}.

Summary of expanded analysis: Tiling cell surface proteins and improved model We repeated the feature exploration for the combined tiling screen (n=2,918 coding sequence targeting perfect match guide RNAs) similar to our initial approach for the GFP screen. Details about the selected features can be found in Example 7, with a full description of features in Table 7. Using these features we derived an updated on-target model (RF_{combined} ) that showed improved correlation to the held-out data during bootstrapping (FIG. 3d ). Accordingly, the RF_{combined} model allowed for the correct assignment of 63% percent of guide RNAs to in the highest-scoring quartile during 10-fold cross-validation (FIG. 3e ), compared to ˜46% for the initial RF_{GFP} model (FIG. 10e ). The RF_{combined} model showed homogenous performance during leave-one-out cross-validation for endogenous genes (mean rₛ=0.63±0.01 ), suggesting that a similar set of features was learned across all three CD protein tiling screens. Importantly, the RF_{combined} model also showed improved performance predicting the outcome of the two fitness screens in HEK293FT and A375 cells (FIG. 3f ).

Taken together, we show that our model can reasonably predict the guide RNA efficiencies, and provide evidence that Cas13d can be used in forward genetic screens. It is important to note that our on-target model (RF_{combined} ) has more predictive power (rₛ=0.67 ) than initial Cas9 on-target models (2014: rₛ=˜0.45, 2016: rₛ=˜0.5 ) 14,15.

We have collected a ˜6× larger dataset to build the improved model. Our improved on-target model (RF_{combined} ) predicts correctly 63% of guides in the highest scoring efficiency quartile, whereas our previous model (RFG_{FP} ) achieved only 46%.

We conducted two targeted fitness screens and included a comparison of depleted genes to previous RNAi screens.

In these screens, we assessed the gene essentiality for 120 endogenous genes. Specifically, we have targeted 10 essential and 10 control genes in human HEK293FT cells and monitored guide depletion 3 predicted low-scoring and 3 predicted high-scoring guides per gene over four weeks. We found that predicted high-scoring guide RNAs targeting essential genes were the most strongly depleted class of guide RNAs (FIGS. 2c and 7a-7b ). Similarly, we targeted 35 essential and 65 control genes in human A375 cells and monitored guide depletion of 20 predicted low-scoring and 20 predicted high-scoring guides per gene over two weeks. Again, in this second cell type (A375 ), we found that predicted high-scoring guide RNAs targeting essential genes were the most strongly depleted class of guide RNAs (FIGS. 2d and 7c-7d ).

In both screens, targeting of control genes (n=75 genes) did not lead to cell dropout (guide RNA depletion) suggesting the observed guide depletions are not mediated by Cas13 off-target activity but are specific to the targeted gene. Accordingly, we found that a gene essentiality score measured by the Cas13 guide RNA depletion correlates to gene essentiality derived by RNAi (DEMETER2, from 712 RNAi screens) with a Spearman rank correlation of up to rₛ=0.71 (FIG. 2f ). However, we also noticed that not all predicted essential genes showed depletion of Cas13 guides (FIG. 7d ), suggesting that RNAi and Cas13 derived results may not be directly comparable. Although the high correlation between RNAi and Cas13 is promising, future studies are under investigation that may systematically compare Cas13 and RNAi in transcriptome-wide fitness screen may shine more light on the touted superiority of Cas13 approaches over RNAi.

### Example 9: Additional Methods and Materials

CRISPR-Cas13 mediates robust transcript knockdown in human cells through direct RNA targeting^{13,15-17.} Compared to DNA-targeting CRISPR enzymes like Cas9, RNA targeting by Cas13 is transcript- and strand-specific: It can distinguish and specifically knock-down processed transcripts, alternatively spliced isoforms and overlapping genes, all of which frequently serve different functions. Previously, we have described a set of optimal design rules for RfxCas13d guide RNAs (gRNAs), and developed a computational model to predict gRNA efficacy for all human protein-coding genes⁴⁶. However, there is a growing interest to target other types of transcripts, such as noncoding RNAs (ncRNAs)^{47,48} or viral RNAs^{49,50} and to target transcripts in other commonly-used organisms^{51, 52, 40,53} In this example, we predicted relative Cas13-driven knock-down for gRNAs targeting messenger RNAs and ncRNAs in six model organisms (human, mouse, zebrafish, fly, nematode and flowering plants) and four abundant RNA virus families (SARS-CoV-2, HIV-1, H1N1 influenza and MERS). To allow for more flexible gRNA efficacy prediction, we also developed a web-based application to predict optimal Cas13d guide RNAs for any RNA target entered by the user.

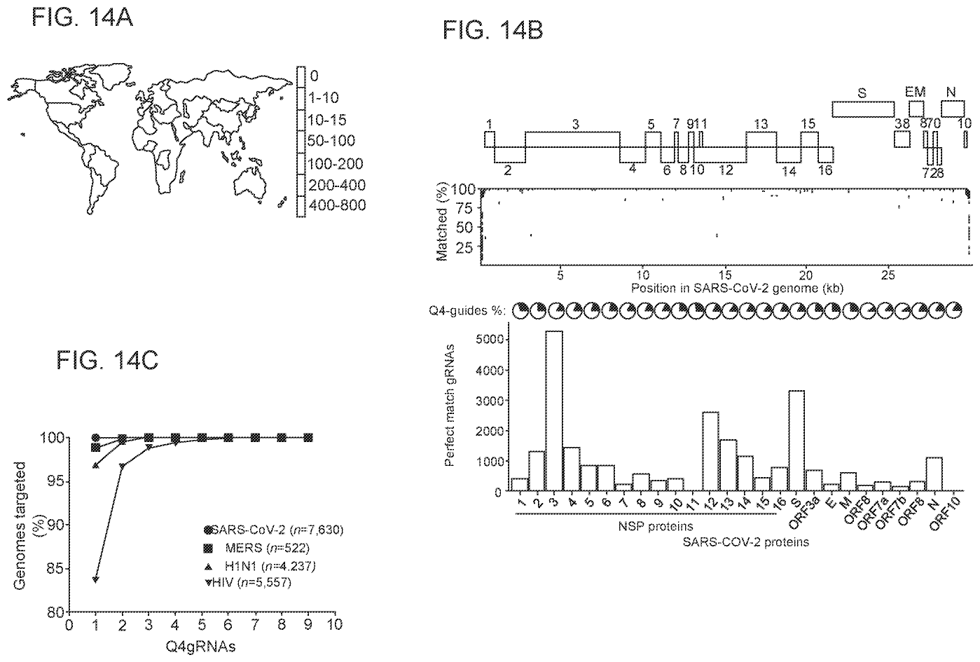

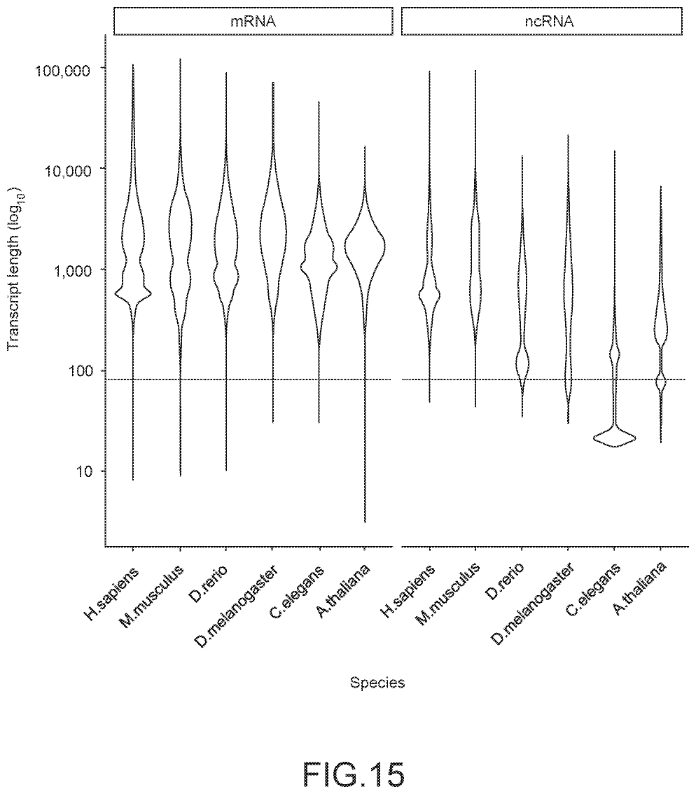

To select optimal gRNAs for transcripts produced from the reference genomes of human, mouse, zebrafish, fly, nematode and flowering plants, we created a user-friendly Cas13 online platform (cas13design.nygenome.org/). We previously found that optimal Cas13 gRNAs depend on specific sequence and structural features, including position-based nucleotide preferences in the gRNA and the predicted folding energy (secondary structure) of the combined direct repeat plus gRNA⁴⁶. Using this algorithm, we pre-computed gRNA efficacies, where possible, for all mRNAs and ncRNAs with varying transcript length for the 6 model organisms (FIG. 14).

For the scored gRNAs for each organism, we found that approximately 20% are ranked in the top quartile (Q4 guides) for both mRNAs and ncRNAs. Remarkably, even though the nucleotide composition can very between RNAs from different species⁵⁴⁻⁵⁶, we find a similar proportion of optimal RfxCas13d gRNAs across all six species.

Next, we examined how many predicted high efficacy gRNAs are present, on average, in different transcripts. To do this, we determined what fraction of the transcripts in each organism include n top-scoring (Q4 ) gRNAs for values of n between 1 and 25. We found that coding sequences contained a higher number of top-scoring gRNA per transcript across all organisms, whereas targeting the noncoding transcriptome is more challenging and varies across different organisms. On average, we were able to find at least 25 Q4 gRNAs for >99% of coding exons in mRNAs but only 80% of ncRNAs. Beyond targeting transcripts from the reference genomes of these model organisms, there are also many other applications of Cas13, such as targeting transcripts from non-model organisms, cleavage of synthetic RNAs, and targeting of transcripts carrying genetic variants not found in the reference genome. Therefore, in addition to these pre-scored gRNAs, we have also developed a graphical interface that allows the user to input a custom RNA sequence for scoring and selection of optimal gRNAs.

Recently, several groups have proposed using CRISPR-Cas13 nucleases to directly target viral RNAs8,⁵⁷ which has become an area of rapid technology development due to the recent COVID-19 pandemic⁵⁸. However, these approaches do not use optimized Cas13 guide RNAs. Previously, we showed that optimal guide RNAs targeting an EGFP transgene can result in a ˜10-fold increase in knock-down efficacy compared to other gRNAs⁴⁶. Therefore, to speed development of effective CRISPR-based antiviral therapeutics, we applied our design algorithm to target SARS-CoV-2 and other serious viral threats using Cas13d.

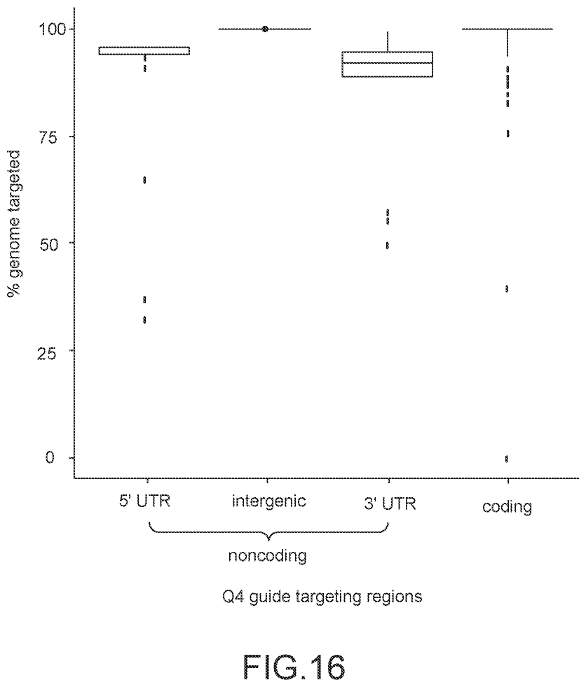

To ensure coverage of diverse patient isolates, we collected 7,630 sequenced SARS-CoV-2 genomes submitted to the Global Initiative on Sharing All Influenza Data (GISAID) database from 58 countries/regions19 (FIG. 14A). Using the first sequenced SARS-CoV-2 isolate from New York City (USA/NY1-PV08001/2020 ) as a reference⁶⁰, we evaluated how many individual SARS-CoV-2 genomes each reference gRNA can target (FIG. 35B). Guide RNAs targeting protein-coding regions are mostly well-conserved across all genomes, with lower conservation in more variable regions such as Non-Structural-Protein 14 (NSP14 ) and Spike (S) protein. We found that gRNAs targeting in the 5′ and 3′ untranslated regions tended to be poorly conserved, as might be expected given the lack of coding function of these regions (FIG. 16). Upon examination of each of the 26 SARS-CoV-2 genes, we found that all gene transcripts could be targeted with Q4 gRNAs.

Similarly, we designed and scored all gRNAs for the coronavirus MERS and two other RNA viruses, HIV-1 which drives Acquired Immunodeficiency Syndrome (AIDS) and H1N1 pandemic influenza. Unlike SARS-CoV-2, where a single high-efficacy (Q4 ) gRNA can target all genomes analyzed, we found that at least two gRNAs are needed to target nearly all available genomes. For the highly mutagenic virus HIV-1⁶¹, we found that nine gRNAs are needed to target all available genomes (FIG. 35C). Given the tremendous interest in viral RNA targeting using Cas13 enzymes, this dataset of optimized gRNAs provides a platform for the development of CRISPR therapeutics for broad targeting of viral populations from diverse patient isolates. All designed gRNAs for model organism and viral transcripts can be interactively browsed or downloaded in bulk on the design tool website.

RNA-targeting CRISPR-Cas13 has great potential for transcriptome perturbation and antiviral therapeutics. In this example, we have designed and scored Cas13d gRNAs for both mRNAs and ncRNAs in six common model organisms and identified optimized gRNAs to target virtually all sequenced viral RNAs for SARS-CoV-2, HIV-1, H1N1 influenza and MERS. We further expanded our web-based platform to make the Cas13 gRNA design readily accessible for model organisms and created a new application to enable gRNA prediction for user-provided target RNA sequences. Given the current lack of Cas13 guide design tools, we anticipate this resource will greatly facilitate CRISPR-Cas13 RNA targeting in model organisms, emerging viral threats to human health and novel RNA targets.

A. gRNA Design for Model Organisms

Reference transcriptomes and corresponding annotations were obtained for each model organism: H. sapiens (GENCODE v19, GRCh37 ), M. musculus (GENCODE M24, mm10 ), D. rerio (Ensembl v99, GRCz11 ), D. melanogaster (Ensembl v99, BDGP6 ), C. elegans (Ensembl v99, WBcel235 ) and A. thaliana (Ensembl Plants v46, TAIR10 ). For each organism, we performed the on-target efficiency predictions for both mRNAs and ncRNAs using command-line RfxCas13d designer version 0.2 as previously described⁴⁶. We scored gRNAs for all RNA targets with a length of at least 80 nucleotides.

B. RNA Virus Genome Collection

All full-length RNA virus genomes were downloaded on Apr. 17, 2020. We downloaded 7,630 complete SARS-CoV-2 viral genomes classified as high coverage and 4,237 Influenza A H1N1 viral genomes with a complete set of eight genomic segments. SARS-CoV-2 and H1N1 genomes were obtained from GISAID (www.gisaid.org/). We also analyzed 522 MERS-CoV and 5,557 full length HIV-1 viral genomes, which were downloaded from NCBI Virus (https://www.ncbi.nlm.nih.gov/labs/virus/).

C. gRNA Design to Target SARS-CoV-2

We split multi-FASTA files into single-entry FASTA files using the UCSC tool faSplit⁶². All possible 23-mer gRNAs targeting individual genomes were scored with the RfxCas13 on-target model described previously⁴⁶. All scored guides were classified into four quartiles. Quartile 4 guides (or Q4 ) are designated to be the predicted best-performing guides. We used USA/NY1-PV08001/2020 (refer to as NY1 isolate) for the SARS-CoV-2 reference gRNA design. Compared to the original (Wuhan) isolate, NY1 contains 3 nucleotide substitutions (G3243A, C25214T, G29027T) resulting in two amino acid mutations (N: A252S, ORF1a: G993S). The SARS-CoV-2 transcript annotation was obtained from NCBI (GenBank: NC_045512.2 ).

D. Prediction of Minimal Numbers of gRNAs to Target RNA Viruses

For each RNA virus, we identified a minimal set of high-scoring Q4 gRNAs that could target all genomes collected. We used a greedy algorithm as described previously⁴⁹: For each iteration, the gRNAs with the highest number of targeting genomes are added to the set. During each iteration, if multiple gRNAs target the same highest number of genomes, we will pick one for the minimal set and start the next iteration.

E. Code Availability

All designed Cas13 guide RNAs (for model organisms and RNA viruses) and the interactive design tool are available at: cas13design.nygenome.org/. The Cas13 guide design algorithm is available at: gitlab.com/sanjanalab/cas13. For additional reproducibility, we provide UNIX and R code to reproduce all figures at: www.dropbox.com/sh/9mk7jlfzcalhnlx/AACp-zkPZSZt6tcLSWOS2a90a?dl=0. Prior to publication, the code to reproduce figures is included on the Gitlab repository. The contents of the websites including the guide sequences referenced in Example 9 are incorporated herein by reference.

Each and every patent, patent application, and publication, including websites cited throughout specification, particularly gitlab.com/sanjanalab/cas13, and the appended Sequence Listing are incorporated herein by reference. While the invention has been described with reference to particular embodiments, it will be appreciated that modifications can be made without departing from the spirit of the invention. Such modifications are intended to fall within the scope of the appended claims.

### REFERENCES

1. Sanjana, N. E., et al. Improved vectors and genome-wide libraries for CRISPR screening. Nat. Methods 11, 783-784 (2014 ).
2. Shalem, O. et al. Genome-Scale CRISPR-Cas9 Knockout Screening in Human Cells. Science (80-.). 343, 84-88 (2014 ).
3. Langmead, B., et al. Ultrafast and memory-efficient alignment of short DNA sequences to the human genome. Genome Biol. 10, (2009 ).
4. Love, M. I., et. al. Moderated estimation of fold change and dispersion for RNA-seq data with DESeq2. Genome Biol. 15, 550 (2014 ).
5. Leek, J. T., et al. The sva package for removing batch effects and other unwanted variation in high-throughput experiments. Bioinformatics 28, 882-883 (2012 ).
6. Kolde, R., et al. Robust rank aggregation for gene list integration and meta-analysis. Bioinformatics 28, 573-580 (2012 ).
7. Lorenz, R. et al. ViennaRNA Package 2.0. Algorithms Mol. Biol. 6, 26 (2011 ).
8. Agarwal, V., et al Predicting microRNA targeting efficacy in Drosophila. Genome Biol. 19, 1-23 (2018 ).
9. Krueger, J. & Rehmsmeier, M. RNAhybrid: microRNA target prediction easy, fast and flexible. Nucleic Acids Res. 34, 451-454 (2006 ).
10. Meeske, A. J. & Marraffini, L. A. RNA Guide Complementarity Prevents Self-Targeting in Type VI CRISPR Systems. Mol. Cell 71, 791-801 (2018 ).
11. Doench, J. G. et al. Rational design of highly active sgRNAs for CRISPR-Cas9-mediated gene inactivation. Nat. Biotechnol. 32, 1262-1267 (2014 ).
12. Abudayyeh, O. O. et al. C2c2 is a single-component programmable RNA-guided RNA-targeting CRISPR effector. Science (80-.). 353, (2016 ).
13. Abudayyeh, O. O. et al. RNA targeting with CRISPR-Cas13. Nature 550, 280-284 (2017 ).
14. East-Seletsky, A. et al. Two distinct RNase activities of CRISPR-C2c2 enable guide-RNA processing and RNA detection. Nature 538, 270-273 (2016 ).
15. Smargon, A. A. et al. Cas13b Is a Type VI-B CRISPR-Associated RNA-Guided RNase Differentially Regulated by Accessory Proteins Csx27 and Csx28. Mol. Cell 65, 618-630 (2017 ).
16. Konermann, S. et al. Transcriptome Engineering with RNA-Targeting Type VI-D CRISPR Effectors. Cell 173, 665-676 (2018 ).
17. Yan, W. X. et al. Cas13d Is a Compact RNA-Targeting Type VI CRISPR Effector Positively Modulated by a WYL-Domain-Containing Accessory Protein. Mol. Cell 70, 327-339 (2018 ). doi:10.1016/j.molcel.2018.02.028
18. Gootenberg, J. S. et al. Nucleic acid detection with CRISPR-Cas13a/C2c2. Science (80 ). 356, 438-442 (2017 ).
19. Gootenberg, J. S. et al. Multiplexed and portable nucleic acid detection platform with Cas13, Cas12a, and Csm6. Science (80-.). 360, 439-444 (2018 ).
20. Cox, D. B. T. et al. RNA editing with CRISPR-Cas13. Science (80 ). 358, 1019-1027 (2017 ).
21. Li, J. et al. Targeted mRNA demethylation using an engineered dCas13b-ALKBH5 fusion protein. bioRxiv (2019 ). doi:10.1101/614859
22. Konermann, S. et al. Transcriptome Engineering with RNA-Targeting Article Transcriptome Engineering with RNA-Targeting. Cell 173, 1-12 (2018 ).
23. Jillette, N. & Cheng, A. W. CRISPR Artificial Splicing Factors. bioRxiv (2018 ). doi:10.1101/431064
24. Anderson, K. M., et al. Targeted Cleavage and Polyadenylation of RNA by CRISPR-Cas13. bioRxiv (2019 ). doi:10.7143/jhep.46.175
25. Yan, W. X. et al. Cas13d Is a Compact RNA-Targeting Type VI CRISPR Effector Positively Modulated by a WYL-Domain-Containing Accessory Protein. Mol. Cell 70, 327-339 (2018 ).
26. Meeske, A. J., et al. Cas13-induced cellular dormancy prevents the rise of CRISPR-resistant bacteriophage. Nature 570, 241-245 (2019 ).
27. Meeske, A. J. & Marraffini, L. A. RNA Guide Complementarity Prevents Self-Targeting in Type VI CRISPR Systems. Mol. Cell 71, 791-801 (2018 ).
28. Zhang, C. et al. Structural Basis for the RNA-Guided Ribonuclease Activity of CRISPR-Cas13d. Cell 175, 212-223 (2018 ).
29. Doench, J. G. et al. Optimized sgRNA design to maximize activity and minimize off-target effects of CRISPR-Cas9. Nat. Biotechnol. 34, 184-191 (2016 ).
30. Zhang, B. et al. Two HEPN domains dictate CRISPR RNA maturation and target cleavage in Cas13d. Nat. Commun. 10, (2019 ).
31. Konermann, S. et al. Genome-scale transcriptional activation by an engineered CRISPR-Cas9 complex. Nature 517, 583-588 (2015 ).
32. Replogle, J. M. et al. Direct capture of CRISPR guides enables scalable, multiplexed, and multi-omic Perturb-seq. bioRxiv (2018 ). doi:10.1101/503367
33. Liu, L. et al. The Molecular Architecture for RNA-Guided RNA Cleavage by Cas13a. Cell 170, 714-720 (2017 ).
34. Tambe, A., et al. RNA Binding and HEPN-Nuclease Activation Are Decoupled in CRISPR-Cas13a. Cell Rep. 24, 1025-1036 (2018 ).
35. Wang, H. et al. CRISPR-mediated live imaging of genome editing and transcription. Science 365, 2-6 (2019 ).
36. Yang, L.-Z. et al. Dynamic Imaging of RNA in Living Cells by CRISPR-Technology. Mol. Cell 76, 1-17 (2019 ).
37. McFarland, J. M. et al. Improved estimation of cancer dependencies from large-scale RNAi screens using model-based normalization and data integration. Nat. Commun. 9, 1-13 (2018 ).
38. Freije, C. A. et al. Programmable Inhibition and Detection of RNA Viruses Using Cas13. Mol. Cell 76, 1-12 (2019 ).
39. Poosala, P., et al. Targeting Toxic Nuclear RNA Foci with CRISPR-Cas13 to Treat Myotonic Dystrophy. bioRxiv 1-29 (2019 ). doi:doi.org/10.1101/716514
40. Mahas, A., et al. CRISPR-Cas13d mediates robust RNA virus interference in plants. Genome Biol. 20, 1-16 (2019 ).
41. Briese, M. et al. A systems view of spliceosomal assembly and branchpoints with iCLIP. Nat. Struct. Mol. Biol. 26, 930-940 (2019 ).
42. Saulière, J. et al. CLIP-seq of eIF4AIII reveals transcriptome-wide mapping of the human exon junction complex. Nat. Struct. Mol. Biol. 19, 1124-1131 (2012 ).
43. Hauer, C. et al. Exon Junction Complexes Show a Distributional Bias toward Alternatively Spliced mRNAs and against mRNAs Coding for Ribosomal Proteins. Cell Rep. 16, 1588-1603 (2016 ).
44. Mukherjee, N. et al. Integrative regulatory mapping indicates that the RNA-binding protein HuR couples pre-mRNA processing and mRNA stability. Mol. Cell 43, 327-39 (2011 ).
45. Lianoglou, S. et al. Ubiquitously transcribed genes use alternative polyadenylation to achieve tissue-specific expression. Genes Dev. 27, 2380-2396 (2013 ).
46. Wessels, H.-H., et al. Massively parallel Cas13 screens reveal principles for guide RNA design. Nature Biotechnology 38, 722-727, (2020 ).
47. Li, S., et al., (January 2020 ) ncRNA-eQTL: a database to systematically evaluate the effects of SNPs on non-coding RNA expression across cancer types. Nucl. Acids. Res., 48 (D1 ):D956-963
48. Xu, D., et al., CRISPR/Cas13-based approach demonstrates biological relevance of vlinc class of long non-coding RNAs in anticancer drug response. Sci Rep 10, 1794, (2020 ).
49. Abbott, T. R., et al. Development of CRISPR as an Antiviral Strategy to Combat SARS-CoV-2 and Influenza. Cell 181, 865-876 e812, (2020 ).
50. Cui, J., et al. Abrogation of PRRSV infectivity by CRISPR-Cas13b-mediated viral RNA cleavage in mammalian cells. Sci Rep 10, 9617, (2020 ).
51. Kushawah, G., et al (January 2020 ) CRISPR-Cas13d induces efficient mRNA knock-down in animal embryos. bioRxiv 10.1101/2020.01.13.904763.
52. Buchman, A. B., et al. Programmable RNA Targeting Using CasRx in Flies. CRISPR J 3, 164-176, (2020 ).
53. Zhou, H., et al. Glia-to-Neuron Conversion by CRISPR-CasRx Alleviates Symptoms of Neurological Disease in Mice. Cell 181, 590-603.e516, (2020 ).
54. Boyle, A. P., et al. Comparative analysis of regulatory information and circuits across distant species. Nature 512, 453-456, (2014 ).
55. Gerstein, M. B., et al. Comparative analysis of the transcriptome across distant species. Nature 512, 445-448, (2014 ).
56. Long, H., et al. Evolutionary determinants of genome-wide nucleotide composition. Nat Ecol Evol 2, 237-240, (2018 ).
57. Blanchard, E. L., et al., (April 2020 ) Treating Influenza and SARS-CoV-2 via mRNA-encoded Cas13a., bioRxiv, p. 1-43, doi: https://doi.org/10.1101/2020.04.24.060418.
58. World Health Organization. WHO Director-General's opening remarks at the media briefing on COVID-19-11 Mar. 2020, www.who.int/dg/speeches/detail/who-director-general-s-opening-remarks-at-the-media-briefing-on-covid-19—11 Mar. 2020>, (2020 ).
59. Shu, Y. & McCauley, J. GISAID: Global initiative on sharing all influenza data—from vision to reality. Euro Surveill 22, (2017 ).
60. Gonzalez-Reiche, A. S., et al. Introductions and early spread of SARS-CoV-2 in the New York City area. Science, (July 2020 ) 369 (6501 ):297-301
61. Cuevas, J. M., et al. Extremely High Mutation Rate of HIV-1 In Vivo. PLoS Biol 13, e1002251, (2015 ).
62. Kuhn, R. M., et al. The UCSC genome browser and associated tools. Brief Bioinform 14, 144-161, (2013 ).

TABLE 10
(Sequence Listing Free Text)
The following information is provided for sequences containing
free text under numeric identifier <223>.

| SEQ ID NO: | |
|---|---|
| (containing free text) | Free text under <223> |
| 1 | DR stem loop |
| 2 | DR stem loop sequence |
| 3 | DR stem loop sequence |
| 4 | DR stem loop sequence |
| 5 | DR stem loop sequence |
| 6 | DR stem loop sequence |
| 7 | DR stem loop sequence |
| 8 | DR stem loop sequence |
| 9 | DR stem loop sequence |
| 10 | DR stem loop sequence |
| 11 | DR stem loop sequence |
| 12 | DR stem loop sequence |
| 13 | DR stem loop sequence |
| 14 | DR stem loop sequence |
| | <222> (1) . . . (1) |
| | <223> B is one of C or G or U |
| 15 | DR stem loop sequence |
| | <222> (1) . . . (1) |
| | <223> N is A or C or G or U |
| | <222> (2) . . . (2) |
| | <223> B is C or G or U |
| 16 | DR stem loop sequence |
| | <222> (1) . . . (1) |
| | <223> N is C or A or G or U |
| | <222> (2) . . . (2) |
| | <223> N is C or A or G or U |
| | <222> (3) . . . (3) |
| | <223> D is A or G or U |
| 17 | DR stem loop sequence |
| | <222> (24) . . . (24) |
| | <223> V is A or C or G |
| 18 | <223> DR stem loop sequence |
| | <222> (23) . . . (23) |
| | <223> V is A or C or G |
| | <222> (24) . . . (24) |
| | <223> N is A or C or G or U |
| 19 | DR stem loop sequence |
| | <222> (22) . . . (22) |
| | <223> V is A or C or U |
| | <222> (23) . . . (23) |
| | <223> N is A or C or G or U |
| | <222> (24) . . . (24) |
| | <223> N is A or C or G or U |
| 20 | DR stem loop sequence |
| 21 | DR stem loop sequence |
| 22 | DR stem loop sequence |
| 23 | DR stem loop |
| | <222> (1) . . . (1) |
| | <223> N is at least one additional |
| | A and/or C and/or G and/or U |
| 24 | DR stem loop |
| | <222> (7) . . . (7) |
| | <223> V is A or C or G |
| 25 | DR stem loop |
| | <222> (1) . . . (1) |
| | <223> B is one of C or G or U |
| | <222> (7) . . . (7) |
| | <223> V is A or C or G |
| 26 | DR stem loop |
| | <222> (1) . . . (1) |
| | <223> N is A or C or G or U |
| | <222> (2) . . . (2) |
| | <223> B is C or G or U |
| | <222> (7) . . . (7) |
| | <223> V is A or C or G |
| 27 | DR stem loop |
| | <222> (1) . . . (1) |
| | <223> N is A or C or G or U |
| | <222> (2) . . . (2) |
| | <223> N is A or C or G or U |
| | <222> (3) . . . (3) |
| | <223> D is A or G or U |
| | <222> (7) . . . (7) |
| | <223> V is A or C or G |
| 28 | DR stem loop |
| | <222> (7) . . . (7) |
| | <223> V is A or C or G |
| | <222> (24) . . . (24) |
| | <223> V is A or C or G |
| 29 | DR stem loop |
| | <222> (7) . . . (7) |
| | <223> V is A or C or G |
| | <222> (23) . . . (23) |
| | <223> V is A or C or G |
| | <222> (24) . . . (24) |
| | <223> N is A or C or G or U |
| 30 | DR stem loop |
| | <222> (7) . . . (7) |
| | <223> V is A or C or G |
| | <222> (22) . . . (22) |
| | <223> H is A or C or U |
| | <222> (23) . . . (23) |
| | <223> N is A or C or G or U |
| | <220> |
| | <221> misc_feature |
| | <222> (24) . . . (24) |
| | <223> N is A or C or G or U |
| 31 | DR stem loop |
| | <222> (6) . . . (6) |
| | <223> V is A or C or G |
| 32 | DR stem loop |
| | <222> (5) . . . (5) |
| | <223> V is A or C or G |
| 33 | DR stem loop |
| | <222> (4) . . . (4) |
| | <223> V is A or C or G |
| 34 | DR stem loop |
| | <222> (1) . . . (1) |
| | <223> N is at least one additional |
| | A and/or C and/or G and/or U |
| | <222> (8) . . . (8) |
| | <223> V is A or G or C |
| 35 | DR stem loop |
| | <222> (18) . . . (18) |
| | <223> D is A or G or U |
| 36 | DR stem loop |
| | <222> (1) . . . (1) |
| | <223> B is one of C or G or U |
| | <222> (18) . . . (18) |
| | <223> D is A or G or U |
| 37 | DR stem loop |
| | <222> (1) . . . (1) |
| | <223> N is A or C or G or U |
| | <222> (2) . . . (2) |
| | <223> B is C or G or U |
| | <222> (18) . . . (18) |
| | <223> D is A or G or U |
| 38 | DR stem loop |
| | <222> (1) . . . (1) |
| | <223> N is A or C or G or U |
| | <222> (2) . . . (2) |
| | <223> N is A or C or G or U |
| | <222> (3) . . . (3) |
| | <223> D is A or G or U |
| | <222> (18) . . . (18) |
| | <223> D is A or G or U |
| 39 | DR stem loop |
| | <222> (18) . . . (18) |
| | <223> D is A or G or U |
| | <222> (24) . . . (24) |
| | <223> V is A or C or G |
| 40 | DR stem loop |
| | <222> (18) . . . (18) |
| | <223> D is A or G or U |
| | <222> (23) . . . (23) |
| | <223> D is A or C or G |
| | <222> (24) . . . (24) |
| | <223> N is A or C or G or U |
| 41 | DR stem loop |
| | <222> (18) . . . (18) |
| | <223> D is A or G or U |
| | <222> (22) . . . (22) |
| | <223> H is A or C or U |
| | <222> (23) . . . (23) |
| | <223> N is A or C or G or U |
| | <222> (24) . . . (24) |
| | <223> N is A or C or G or u |
| 42 | DR stem loop |
| | <222> (17) . . . (17) |
| | <223> D is A or G or U |
| 43 | DR stem loop |
| | <222> (16) . . . (16) |
| | <223> D is A or G or U |
| 44 | DR stem loop |
| | <222> (15) . . . (15) |
| | <223> D is A or G or U |
| 45 | DR stem loop |
| | <222> (1) . . . (1) |
| | <223> N is at least one additional |
| | A and/or C and/or G and/or U |
| | <222> (19) . . . (19) |
| | <223> D is A or G or U |
| 46 | DR stem loop |
| 47 | FLAG tag |
| 48 | Amino acid sequence of the Cas |
| | 13d variant |

1. A Class 2, Type VI clustered regularly interspaced short palindromic repeat (CRISPR) RNA (crRNA) which comprises a direct repeat (DR) stem loop sequence and a guide or spacer sequence, said DR selected from one or more of the following DR sequences or a modification thereof:

| DR Ref | | SEQ ID |
|---|---|---|
| No. | DR Sequence | NO: |
| (I) | AACCCCUACCAACUGGUCGGGGUUUGAAAC | 1 |
| (II) | UACCCCUACCAACUGGUCGGGGUUUGAAAC | 2 |
| (III) | UUCCCCUACCAACUGGUCGGGGUUUGAAAC | 3 |
| (IV) | UUUCCCUACCAACUGGUCGGGGUUUGAAAC | 4 |
| (V) | UUACCCUACCAACUGGUCGGGGUUUGAAAC | 5 |
| (VI) | AACCCCGACCAACUGGUCGGGGUUUGAAAC | 6 |
| (VII) | UACCCCGACCAACUGGUCGGGGUUUGAAAC | 7 |
| (VIII) | UUCCCCGACCAACUGGUCGGGGUUUGAAAC | 8 |
| (IX) | UUUCCCGACCAACUGGUCGGGGUUUGAAAC | 9 |
| (X) | AACCCCUACCAACUGGUAGGGGUUUGAAAC | 10 |
| (XI) | UACCCCUACCAACUGGUAGGGGUUUGAAAC | 11 |
| (XII) | UUCCCCUACCAACUGGUAGGGGUUUGAAAC | 12 |
| (XIII) | UUUCCCUACCAACUGGUAGGGGUUUGAAAC | 13 |
| (IIₐₗₜ₁) | BACCCCUACCAACUGGUCGGGGUUUGAAAC | 14 |
| (IIIₐₗₜ₁) | NBCCCCUACCAACUGGUCGGGGUUUGAAAC | 15 |
| (IVₐₗₜ₁) | NNDCCCUACCAACUGGUCGGGGUUUGAAAC | 16 |
| (Vₐₗₜ₁) | AACCCCUACCAACUGGUCGGGGUVUGAAAC | 17 |
| (VIₐₗₜ₁) | UUACCCUACCAACUGGUCGGGGVNUGAAAC | 18 |
| (VIIₐₗₜ₁) | AACCCCUACCAACUGGUCGGGHNNUGAAAC | 19 |
| (VIIIₐₗₜ₁) | -ACCCCUACCAACUGGUCGGGGUUUGAAAC | 20 |
| (IXₐₗₜ₁) | --CCCCUACCAACUGGUCGGGGUUUGAAAC | 21 |
| (Xₐₗₜ₁) | ---CCCUACCAACUGGUCGGGGUUUGAAAC | 22 |
| (XIIₐₗₜ₁) | (N)ₙAACCCCUACCAACUGGUCGGGGUUUGAAAC | 23 |
| (Iₐₗₜ₂) | AACCCCVACCAACUGGUCGGGGUUUGAAAC | 24 |
| (IIₐₗₜ₂) | BACCCCVACCAACUGGUCGGGGUUUGAAAC | 25 |
| (IIIₐₗₜ₂) | NBCCCCVACCAACUGGUCGGGGUUUGAAAC | 26 |
| (IVₐₗₜ₂) | NNDCCCVACCAACUGGUCGGGGUUUGAAAC | 27 |
| (Vₐₗₜ₂) | AACCCCVACCAACUGGUCGGGGUVUGAAAC | 28 |
| (VIₐₗₜ₂) | UUACCCVACCAACUGGUCGGGGVNUGAAAC | 29 |
| (VIIₐₗₜ₂) | AACCCCVACCAACUGGUCGGGHNNUGAAAC | 30 |
| (VIIIₐₗₜ₂) | -ACCCCVACCAACUGGUCGGGGUUUGAAAC | 31 |
| (IXₐₗₜ₂) | --CCCCVACCAACUGGUCGGGGUUUGAAAC | 32 |
| (Xₐₗₜ₂) | ---CCCVACCAACUGGUCGGGGUUUGAAAC | 33 |
| (XIIₐₗₜ₂) | (N)ₙAACCCCVACCAACUGGUCGGGGUUUGAAAC | 34 |
| (Iₐₗₜ₃) | AACCCCUACCAACUGGUDGGGGUUUGAAAC | 35 |
| (IIₐₗₜ₃) | BACCCCUACCAACUGGUDGGGGUUUGAAAC | 36 |
| (IIIₐₗₜ₃) | NBCCCCUACCAACUGGUDGGGGUUUGAAAC | 37 |
| (IVₐₗₜ₃) | NNDCCCUACCAACUGGUDGGGGUUUGAAAC | 38 |
| (Vₐₗₜ₃) | AACCCCUACCAACUGGUDGGGGUVUGAAAC | 39 |
| (VIₐₗₜ₃) | UUACCCUACCAACUGGUDGGGGVNUGAAAC | 40 |
| (VIIₐₗₜ₃) | AACCCCUACCAACUGGUDGGGHNNUGAAAC | 41 |
| (VIIIₐₗₜ₃) | -ACCCCUACCAACUGGUDGGGGUUUGAAAC | 42 |
| (IXₐₗₜ₃) | --CCCCUACCAACUGGUDGGGGUUUGAAAC | 43 |
| (Xₐₗₜ₃) | ---CCCUACCAACUGGUDGGGGUUUGAAAC | 44 |
| (XIIₐₗₜ₃) | (N)ₙAACCCCUACCAACUGGUDGGGGUUUGAAAC | 45 |

wherein R represent A or G; Y represents C or T(or U); S represents G or C; W represents A or T(or U); K represents G or T(or U); M represents A or C; B represents C or G or T(or U); D represents A or G or T(or U); H represents A or C or T(or U); V represents A or C or G; N represents any base; and - represents a nucleotide gap.

2. The cRNA according to claim 1, which is a non-naturally occurring, chemically synthesized, recombinantly synthesized or engineered crRNA.

3. The crRNA according to claim 1 or 2, which is capable of forming a complex with a Class 2, Type VI effector protein, and directing the complex to bind to the target RNA to cleave or block the target RNA.

4. The cRNA according to any of claim 3, wherein the Class 2, Type VI effector protein is a CRISPR-associated protein 13d (Cas13d).

5. The cRNA according to claim 4, wherein the Cas13d protein is selected from a RfxCas13d from Ruminococcus flavefaciens strain XPD3002, an AdmCas13d from Anaerobic digester metagenome 15706, EsCas13d from Eubacterium siraeum DSM15702, P1E0Cas13d from Gut metagenome assembly P1E0-k21, UrCas13d from Uncultured Ruminoccocus sp., RffCas13d from Ruminoccocus flavefaciens FD1, and RaCas13d from Ruminoccocus albus, or a Cas13d that shares at least 80% homology with the RNA sequence of RfxCas13d.

6. A nucleic acid molecule comprising a crRNA of any of claims 1 to 5.

7. The nucleic acid molecule according to claim 6, which is a mature RNA.

8. The nucleic acid molecule according to claim 6, further comprising a DNA sequence encoding a Class 2, Type VI effector protein or a variant thereof under the control of a first regulatory sequence directing expression thereof in a mammalian cell.

9. The nucleic acid molecule according to claim 8, wherein the first regulatory sequence comprises an RNA polymerase II (Pol II) promoter.

10. The nucleic acid molecule according to claim 9, wherein
(a) the Pol II promoter is an EF-1 alpha short (EFS) promoter, or a Tet operator (tetO) promoter;
(b) the first regulatory sequence comprises one or more of a polyadenylation sequence, a selectable marker, a tag, and an optional Woodchuck Hepatitis Virus Posttranscriptional Regulatory Element sequence; or
(c) the tag is selected from one or more of the following: a FLAG tag, a poly(His) tag, a chitin binding protein (CBP) tag, a maltose binding protein (MBP) tag, a Strep tag, a glutathione-S-transferase (GST) tag, a thioredoxin (TRX) tag, a poly(NANP) tag, a V5 tag, a HA tag, a Spot tag, a T7 tag, a NE tag, a fluorescence tag, a Green Fluorescent Protein (GFP) tag, and a MYC tag; or
(d) the selectable marker is selected from a a puromycin resistance gene, a kanamycin resistance gene, a chloramphenicol resistance gene, a blasticidin S resistance gene, a geneticin resistance gene, a hygromicin resistance gene, an ampicillin resistance gene, a tetracycline resistance gene, and a G418 resistance gene.

11. The nucleic acid molecule according to any one of claims 6 through 10, wherein the molecule further comprises a second regulatory sequence directing expression of the crRNA.

12. The nucleic acid molecule according to claim 11, wherein the second regulatory sequence comprises an RNA polymerase III (Pol III) promoter.

13. The nucleic acid molecule according to claim 12, wherein the Pol III promoter is selected from a U6 promoter, a H1 promoter, a T7 promoter and a 7SK promoter.

14. The nucleic acid molecule according to any one of claims 6 to 13, which is a vector or plasmid.

15. The nucleic acid molecule according to claim 14, wherein said vector is a viral vector.

16. The nucleic acid molecule according to claim 15, wherein the viral vector is a retrovirus vector, a lentivirus vector, an adenovirus vector, an adeno-associated virus vector, or a hybrid viral vector.

17. The cRNA or nucleic acid molecule according to any one of claims 1 to 16, wherein the Cas13d or a variant thereof further comprises a nuclear localization signals (NLS) or a cytosolic signal or a nuclear-export signal (NES).

18. The cRNA or nucleic acid molecule according to claim 17, wherein the Cas13d or a variant thereof is fused to an endoplasmic reticulum localization element, an Outer Mitochondrial membrane localization element, a Mitochondria localizing element, a Nucleolus localizing element (NIK3x), a Nuclear lamina localizing element (LMNA) or a Nuclear pore complex localizing element (SENP2 ).

19. The crRNA or nucleic acid molecule according to any one of claims 1 to 18, wherein the crRNA has the DR sequence of SEQ ID NO: 2, 14, 25 or 36.

20. The cRNA or nucleic acid molecules according to any one of claims 1 to 19, in which the crRNA comprises a guide sequence which mismatches the target and allows the Class 2, Type VI effector protein to bind the target, but not elicit target degradation.

21. A ribonucleoprotein (RNP) complex comprising a Class 2, Type VI effector protein and a crRNA of claim 1.

22. A composition comprising a cRNA, nucleic acid molecule or RNP complex of any of claims 1 to 21 in a pharmaceutically acceptable carrier.

23. The composition according to claim 22, wherein said carrier is a nanoparticle, a lipid complex, a polymer, a quantum dot, a carbon nanotube, a magnetic nanoparticle, or a gold nanoparticle.

24. A cell comprising any of the cRNA, nucleic acid molecules, RNP or compositions of claims 1 through 23.

25. A library comprising a plurality of crRNAs, nucleic acid molecules, viral vectors or RNPs according to any one of claims 1 to 23, wherein each of the crRNA is capable of directing a Cas13d or a variant thereof to a different target RNA or a different region of one target RNA.

26. A pharmaceutical composition comprising a crRNA, nucleic acid molecule, RNP, composition, cell or library of any of claims 1 to 25.

27. The pharmaceutical composition according to claim 26, for use in the treatment of a disease associated with an abnormal RNA or misregulation of an RNA transcript.

28. The RNP, pharmaceutical compositions, cells and libraries of any one of claims 21-27, in which the crRNA RNAs comprise guide sequences which mismatch the target and allow the Class 2, Type VI effector protein to bind the target, but not elicit target degradation.

29. A method of treating a disease associated with an abnormal RNA or misregulation of an RNA transcript, comprising administering to a subject in need thereof the pharmaceutical composition of claim 26.

30. The method according to claim 29, wherein the administering step comprises
(a) delivering the crRNA of claim 1 as a mature RNA to a cell that has been conditioned to express an RNA-targeting CRISPR-associated protein;
(b) delivering the crRNA of claim 1 in a vector which co-expresses the RNA-targeting CRISPR-associated protein; and
(c) delivering the crRNA and RNA-targeting CRISPR-associated protein as a ribonucleoprotein complex or
(d) delivering the nucleotide molecule of claim 11 with a separate nucleotide molecule that expresses the RNA-targeting CRISPR-associated protein.

31. The method according to claim 29, wherein the disease is selected from a cancer/tumor, a solid tumor (e.g. breast, colon, ovarian, lung, liver and glioma), a B cell lymphoma, a Lymphoid leukaemia, HCV infection and related liver Diseases, Atherosclerosis, Polycystic Kidney Disease, Cardiac disease, Cardiac stress, Myocardial infarction, Kidney fibrosis, Cardiac fibrosis, diabetes, Diabetes-related kidney complications, type 2 diabetes, non-alcoholic fatty liver diseases, Cutaneous T cell lymphoma and mycosis fungoides, Scleroderma, Mesothelioma, Sickle Cell anemia, Spinal muscular atrophy, and non-small cell lung cancer.

32. A method of improving the efficiency of targeting or stabilization of a Class 2, Type VI clustered CRISPR RNA (crRNA) which comprises a direct repeat (DR) stem loop and a guide or spacer sequence, said method comprising replacing the DR stem loop sequence of said crRNA with a DR sequence selected from one or more of the DR sequences of SEQ ID Nos: 1 to 46, or a modification thereof.

33. A method for screening or predicting on-target activity of a clustered regularly interspaced short palindromic repeats (CRISPR) RNA (crRNA), whereby the crRNA is capable of forming a complex with an RNA-targeting CRISPR-associated protein or a variant thereof and directing the complex to the target RNA, said method comprising
(a) characterizing a plurality of crRNAs and their corresponding target by features comprising the presence of both a seed region located between guide RNA nucleotide bases 15 to 21 relative to the guide RNA 5′ end, characterized by a stabilizing, enriched sequence of G and C bases and an accessible target region characterized by an enriched sequence of A and U, surrounding the seed region on the 5′ end, 3′ end or both the 5′ and 3′ ends;
(b) assessing on-target activity of each of the crRNAs of (a);
(c) applying a machine learning model or deep learning model using the characterization of (a) and the on-target activity of (b), wherein input of the model comprises characterization(s) of said seed region and target regions of each crRNA and its corresponding target RNA, wherein output of the model is an on-target score of the crRNA, and wherein a higher score indicates a ranked on-target activity;
(d) applying the model constructed in step (c) to a first crRNA and generating an on-target score of the first crRNA.

34. The method according to claim 33, wherein the features of crRNA(s) and the corresponding target RNA(s) in step (a) are selected from any combination of
i. at least the top 1, 2, 5, 10, 15, 20, 25, 30, 35 or more features of Table 5;
ii. any combination of 2 or more of the features of Table 5,
iii. at least the top 1, 2, 3, 4, 5, or 6 features of the RF_{GFP} features listed in Table 2,
iv. at least the top 1, 2, 5, 10, 15, 20, 25, 30, or 33 features of the RF_{combined} features listed in Table 2;
v. any combination of 2 or more features listed in Table 2 and
vi. having a DR sequence of Table 9.

35. The method according to any one of claims 33 to 34 wherein step (d) further comprises applying the model constructed in step (c) to a second crRNA having the same target RNA, and generating an on-target score of the second crRNA.

36. The method according to any one of claims 33 to 35, wherein an on-target activity of step (b) is one or more of the following:
efficacy of the crRNA in forming a complex with a Cas13d protein or a variant thereof;
efficacy of the crRNA in hybridizing to the corresponding target RNA;
efficacy of the crRNA in directing a Cas13d-crRNA complex to the target RNA;
efficacy of the crRNA in reducing the corresponding target RNA after hybridizing to the target RNA;
enrichment or depletion of the CRISPR pooled screen readout;
efficacy of the guide of the crRNA or the target RNA after applying the crRNA and a Cas13d or a variant thereof to a cell or cell culture, a non-human organism or an in vitro, cell-free assay system; or
efficacy of the crRNA comprising guide sequences which mismatch the target, to allow the Class 2, Type VI effector protein to bind the target, but not elicit target degradation.

37. The method according to claim 33, wherein on-target activity includes binding without cleavage.

38. The method according to any one of claims 33 to 37, wherein the target RNA is a messenger RNA (mRNA), a mature mRNA, a primary transcript mRNA (pre-mRNA), a ribosomal RNA (rRNA), a 5.8S rRNA, a 5S rRNA, a transfer RNA (tRNA), a transfer-messenger RNA (tmRNA), an enhancer RNA (eRNA), a small interfering RNA (siRNA), a microRNA (miRNA), a small nucleolar RNA (snoRNA), a Piwi-interacting RNA (piRNA), a tRNA-derived small RNA (tsRNA), a small rDNA-derived RNA (srRNA), a non-coding RNA (ncRNA), long (intergenic) non-coding RNA (lincRNA/lncRNA), a single-stranded RNA (ssRNA), a circular RNA (circRNA), a vault RNA (vRNA/vtRNA), a SmY RNA, a double-stranded RNA (dsRNA), a small Cajal body-specific RNA (scaRNA), an antisense RNA (aRNA/asRNA), a ribonuclease RNA (e.g. RNase P), a non-coding regulatory RNA (e.g. 7SK RNA), RNA-viruses, single stranded DNA, coding sequence (CDS) of a RNA, 5′ untranslated region (UTR) of a RNA, 3′ UTR of a RNA, or a intron of a RNA, or a satellite repeat sequence embedded in any of said RNA targets.

39. The method of any one of claims 33-38, wherein the crRNA guides are characterized by the DR sequences of claim 1.

40. The method according to claim 33, in which the crRNA comprises a guide sequence which mismatches the target and allows the Class 2, Type VI effector protein to bind the target, but not elicit target degradation.

41. A method of blocking RNA regulatory elements without degradation of the target by administering to a cell expressing an RNA-targeting CRISPR-associated protein or to a subject, crRNAs capable of forming a complex with the RNA-targeting CRISPR-associated protein or a variant thereof and directing the complex to the target RNA, wherein said crRNAs comprise a DR sequence and a guide or spacer sequences, said guide or spacer sequences forming extended mismatches to the target site in the seed region.

42. The method according to claim 41, wherein the seed region is located between guide RNA nucleotide bases 15 to 21 relative to the guide RNA 5′ end, characterized by a stabilizing, enriched sequence of G and C bases and an accessible target region characterized by an enriched sequence of A and U, surrounding the seed region on the 5′ end, 3′ end or both the 5′ and 3′ ends.

43. The method according to claim 41 or 42 wherein the extended mismatches include 4 of more mismatched bases in the seed region.

44. The method according to claim 41, wherein the crRNAs are designed and selected by use of the method of any of claims 33-37.

45. The method according to claim 41, wherein the crRNAs each comprise at least one DR of Table 9.

46. A method for targeting multiple RNA targets comprising:
(a) administering to a cell expressing an RNA-targeting CRISPR-associated effector protein or to a subject crRNAs capable of forming a complex with the RNA-targeting CRISPR-associated effector protein or a variant thereof and directing the complex to a primary target RNA, wherein said crRNAs comprise a guide or spacer sequence that is a perfect match for the seed region of the target; and
(b) administering to the cell expressing the same RNA-targeting CRISPR-associated effector protein or to a subject crRNAs capable of forming a complex with said RNA-targeting CRISPR-associated effector protein or a variant thereof and directing the complex to a secondary target RNA, wherein said crRNAs comprise a guide or spacer sequence that forms extended mismatches to the secondary target site in the seed region.

47. The method according to claim 46, wherein the effector protein is a Class 2, Type VI effector protein.

48. The method according to claim 47, wherein the effector protein is Cas13d or a variant or ortholog thereof.

49. The method according to claims 46 to 48, wherein the primary target is cleaved and the secondary target is blocked without being degraded.
